(19) United States
(12) Patent Application Publication
(10) Pub. No.: US 2022/0049285 A1
(43) Pub. Date: Feb. 17, 2022

(54) SINGLE CELL/EXOSOME/VESICLE PROTEIN PROFILING

(71) Applicant: The Trustees of Columbia University in the City of New York, New York, NY (US)

(72) Inventors: Alejandro Chavez, New York, NY (US); Jiemin Sheng, New York, NY (US)

(73) Assignee: The Trustees of Columbia University in the City of New York, New York, NY (US)

(21) Appl. No.: 17/397,454

(22) Filed: Aug. 9, 2021

Related U.S. Application Data
(63) Continuation-in-part of application No. PCT/US2020/017327, filed on Feb. 7, 2020.
(60) Provisional application No. 62/962,788, filed on Jan. 17, 2020.
(60) Provisional application No. 62/945,533, filed on Dec. 9, 2019.
(60) Provisional application No. 62/803,067, filed on Feb. 8, 2019.

(51) Int. Cl. C12Q 1/6804

(52) U.S. Cl. CPC C12Q 1/6804

(57) ABSTRACT

The present disclosure is directed, at least in part, to methods and systems for quantifying the levels of multiple, e.g., over a hundred or more, target molecules, e.g., proteins, in, or on the surface of, single entities, including single exosomes, single cells or single vesicles.

### RELATED APPLICATIONS

This application claims the benefit of priority to International Application No. PCT/US2020/017327, filed Feb. 7, 2020, which claims the benefit of priority to U.S. Provisional Patent Application No. 62/803,067, filed Feb. 8, 2019, U.S. Provisional Patent Application No. 62/945,533, filed Dec. 9, 2019, and U.S. Provisional Patent Application No. 62/962,788, filed Jan. 17, 2020. The content of these applications is incorporated herein by reference in their entirety.

### SEQUENCE LISTING

The instant application contains a Sequence Listing which has been submitted electronically in ASCII format and is hereby incorporated by reference in its entirety. Said ASCII copy, created on Oct. 20, 2021, is named 123756-01204_SL.txt and is 56,310 bytes in size.

### BACKGROUND

The treatment of disease begins with an accurate and timely diagnosis of a patient. Many diagnoses are performed by examining well-known surrogate markers that can be assayed for in routine laboratory tests, such as white blood cell counts or serum analyte levels. Often, test results combined with experience and physical symptoms may be enough for the physician to make a confident diagnosis. However, there are still many instances where biopsies are used to confirm these diagnoses. Biopsies are, by nature, physically invasive and time consuming to perform. Even after diagnosis and treatment, physicians often have to revisit their hypotheses based on a patient's response to treatment. Follow-up exams, much like primary diagnostics, may rely heavily upon surrogate markers that do not provide adequate biological insight into the body's current state. Now with the increased realization that the course of disease and response to treatment is not uniform, which has prompted the adoption of so-called personalized medicine, real-time tissue health monitoring are needed. However, there is still a lack of non-invasive technologies that enable near real-time readout of tissue state.

Recent single-cell RNA-seq (scRNA-seq) methodology has improved the ability of researchers to study cellular composition and heterogeneity due to its unbiased and high-throughput nature. New scRNA-seq based methods (e.g., CITE-seq, REAP-seq) can produce digital, sequencing-based readout for protein levels by conjugating antibodies to oligonucleotides that contain a barcode for antibody identification. However, these methods require access and familiarity with specialized equipment (e.g., mass cytometry (CyTOF), immunofluorescence microscopy, and/or flow cytometry) for single-cell library generation and do not use unique molecular identifiers (UMIs).

Cell surface proteins, which make up the surfaceome, have been extensively cataloged for their role in development (Collier, A. et al (2017). Comprehensive Cell Surface Protein Profiling Identifies Specific Markers of Human Naive and Primed Pluripotent States. Cell Stem Cell, 20(6), 874-890; Wojdyla, K., et al. (2020). Cell-Surface Proteomics Identifies Differences in Signaling and Adhesion Protein Expression between Naive and Primed Human Pluripotent Stem Cells. Stem Cell Reports, 14(5), 972-988), cell-cell interactions (Maurel, D., et al. (2008). Cell-surface protein-protein interaction analysis with time-resolved FRET and snap-tag technologies: application to GPCR oligomerization. Nature Methods, 5(6), 561-567.), and signal transduction (Kabbani, N. (2008). Proteomics of membrane receptors and signaling. Proteomics, 8(19), 4146-4155). Surfaceomes are known to change in disease states and are important for not only disease identification, but also for understanding their biological basis (Ghosh, D., et al (2017). A Cell-Surface Membrane Protein Signature for Glioblastoma. Cell Systems, 4(5), 516-529 2017; Leung, K. K., et al (2020). Broad and thematic remodeling of the surfaceome and glycoproteome on isogenic cells transformed with driving proliferative oncogenes. PNAS, 117(14), 7764-7775). In a clinical context, the ability to distinguish the quantity of important protein markers on patient cells with precision and accuracy is critical for disease diagnosis (Jaye, D. L., et al (2012). Translational Applications of Flow Cytometry in Clinical Practice. The Journal of Immunology, 188, 4715-4719; Wood, B. L., et al (2006). 2006 Bethesda International Consensus recommendations on the immunophenotypic analysis of hematolymphoid neoplasia by flow cytometry: Optimal reagents and reporting for the flow cytometric diagnosis of hematopoietic neoplasia. Cytometry Part B (Clinical Cytometry), 72B(S1), 514-522). Furthermore, the importance of cell surface proteins is highlighted by the fact that ˜60-70% of modern pharmaceuticals target cell surface proteins and over a quarter of human genes code t for membrane proteins (Kuhlmann, L., et al (2018). Cell-surface proteomics for the identification of novel therapeutic targets in cancer. Expert Review of Proteomics, 15(3); Ye, X., et al (2020). Cell surface protein enrichment for biomarker and drug target discovery using mass spectrometry-based proteomics. In Proteomic and Metabolomic Approaches to Biomarker Discovery (2nd ed., pp. 409-420). Academic Press). Though studies have been undertaken to catalog the bulk composition of cell surfaceomes, such population-averaged measurements often fail to detect rare cell types or states which may play meaningful biological roles. Thus, the ability to measure the surface proteomes on single cells in heterogeneous populations remains an important goal (Labib, M., & Kelley, S. O. (2020). Single-cell analysis targeting the proteome. Nature Reviews Chemistry, 4(3), 143-158).

Traditionally flow cytometry has been the method of choice for analyzing proteins present on single cells because of its high throughput capacity and well-benchmarked standards (Jaye, D. L., et al (2012). Translational Applications of Flow Cytometry in Clinical Practice. The Journal of Immunology, 188, 4715-4719). However, flow cytometry has limitations stemming from the spectral overlap of fluorescent conjugates, requiring custom built antibody panels with the ability to interrogate only a limited number of protein targets at a time. This can be particularly problematic in situations where sample cells are scarce, allowing only a limited number of proteins of interest to be examined Additionally, even in cases where the cell sample is plentiful, relationships between proteins are often incomplete, as not all antigens can be assayed at the same time, limiting study to only known relationships and potentially missing novel associations predictive of disease outcome or indicative of novel biological processes (Labib & Kelley, 2020). Though mass cytometry overcomes some of these barriers, it has not seen wide adoption because of the need for highly specialized equipment (Behbehani, G. K., et al (2012). Single-cell mass cytometry adapted to measurements of the cell cycle. Cytometry Part A, 81A(7), 552-566; Palii, C. G., et al (2019, May). Single-Cell Proteomics Reveal that Quantitative Changes in Co-expressed Lineage-Specific Transcription Factors Determine Cell Fate. Cell Stem Cell, 24(5), 812-820).

More recently, new techniques have been developed to push the envelope of protein detection in single cells (Lin, J., et al (2019). Ultra-sensitive digital quantification of proteins and mRNA in single cells. Nature Communications, 10(1), 3544; Stoeckius, M., et al. (2017). Simultaneous epitope and transcriptome measurement in single cells. Nature Methods, 14(9), 865-868; Peterson, V. M., et al (2017). Multiplexed quantification of proteins and transcripts in single cells. Nature Biotechnology, 35(10), 936-939; Mimitou, E. P., et al (2019). Multiplexed detection of proteins, transcriptomes, clonotypes and CRISPR perturbations in single cells. Nature Methods, 16(5), 409-412; Hwang, B., Lee, et al (2021). SCITO-seq: single-cell combinatorial indexed cytometry sequencing. Nature Methods, 18(8), 903-91). Many of these techniques seek to leverage the emerging accessibility and sensitivity of next generation sequencing (NGS) for accurate biomolecule (DNA, RNA, protein) detection in single cells. Previous methods aimed at leveraging NGS for detection of proteins on single cells have shown that staining and sequencing cells with DNA barcoded antibodies allows for quantitative readouts of protein abundance on single cells (Stoeckius, M., et al (2017). Simultaneous epitope and transcriptome measurement in single cells. Nature Methods, 14(9), 865-868; Peterson et al., 2017; Mimitou et al., 2019). However, a large barrier to adoption of these techniques is the complex instrumentation necessary for making single cell suspensions, which adversely affects both cost and accessibility.

Thus, there is a need for noninvasive and near real-time readout to quantify the abundance and state of proteins in a cellular or exosomal sample isolated from a patient before and after treatment using technologies that do not require in-house specialized equipment and have the ability to scale to hundreds of protein targets in a patient sample.

### SUMMARY OF THE DISCLOSURE

The present disclosure is directed, at least in part, to methods and systems for accurately quantifying the levels of multiple, e.g., over a hundred or more, target molecules, e.g., proteins, in, or on the surface of, single entities, including single exosomes, single cells or single vesicles. In some embodiments, the disclosure provides methods for high throughput detection of a plurality of target molecules within pooled single exosomes, single cells or single vesicles using DNA-barcoded antibodies (DBAs), split-pool sequencing, and optionally unique molecular identifiers (UMIs). The methods and systems of the disclosure require no specialized equipment to scale to hundreds of protein targets, thereby providing cost-effective, modular and easy-to-use quantitative single cell protein profiling platform.

The methods and systems of the disclosure can be applied to early stage discovery pipelines to identify pathways of interest (e.g., in a preclinical setting) and in clinical settings for diagnostic and/or therapeutic applications for a variety of diseases and disorders. For example, the methods and systems of the disclosure can be applied to identify which patients with, for example, cancer, autoimmune disease or inflammatory disease are more likely to respond to any of a diverse set of therapeutics, e.g., immune regulators, such as, for example, anti-PD1 antibodies).

Accordingly, in some aspects, the present disclosure is directed to methods for detecting a plurality of target molecules in, or on the surface of, entities such as exosomes, cells or vesicles in a sample, the method comprising: (a) contacting the entities such as exosomes, cells or vesicles with a plurality of target molecule-binding agents, wherein each target molecule-binding agent comprises a nucleic acid barcode and optionally a unique molecular identifier (UMI), and wherein target molecule-binding agents that are specific to an identical target molecule share an identical nucleic acid barcode.

In some aspects, the present disclosure is directed to methods for diagnosing a disease or disorder, e.g., cancer, autoimmune disease or inflammatory disease, determining a patient's response to a therapy, and/or monitoring the patient for undesired side effects to the therapy, the method comprising: (a) contacting exosomes, cells or vesicles with a plurality of target molecule-binding agents, wherein each target molecule-binding agent comprises a nucleic acid barcode and optionally a unique molecular identifier (UMI), wherein target molecule-binding agents that are specific to an identical target molecule share an identical nucleic acid barcode, wherein the exosomes, cells or vesicles are isolated from a patient suffering from a disease or disorder, e.g., cancer, autoimmune disease or inflammatory disease, wherein the plurality of target molecules comprise molecules that are markers indicative of a disease or disorder, e.g., cancer, autoimmune disease or inflammatory disease, efficacy of the therapy, and/or are indicative of undesirable side effects, and wherein expression levels of the target molecules determined in the patient are compared with expression levels of the corresponding target molecules determined in normal controls, thereby diagnosing a disease or disorder, e.g., cancer, autoimmune disease or inflammatory disease, determining the patient's response to a therapy, and/or monitoring for undesired side effects of a therapy.

In some embodiments, the markers are tested before and after treatment with a therapy.

In some embodiments, the patient's response to therapy is determined and/or monitored in real-time.

In some embodiments, the patient is further treated with the same therapy, if the therapy is effective, but associated with little or no undesired side effects; or the patient is treated with a different therapy if the therapy is not effective and/or is associated with undesired side effects.

In some embodiments, each target molecule-binding agent described herein above further comprises a universal round 1 primer sequence at the 3′ end for a first round extension.

In some embodiments, the methods described herein further comprises:
(b) dividing the exosomes, cells or vesicles into at least two primary aliquots, the at least two primary aliquots comprising at least a first primary aliquot and a second primary aliquot;
(c) adding primary nucleic acid tags to the target molecule-binding agents in the at least two primary aliquots, wherein the primary nucleic acid tags added to the target molecule-binding agents in any one of the at least two primary aliquots are different from the primary nucleic acid tags added to the target molecule-binding agents in any one of the other primary aliquots;
(d) combining the at least two primary aliquots;
(e) dividing the combined primary aliquots into at least two secondary aliquots, the at least two secondary aliquots comprising a first secondary aliquot and a second secondary aliquot;
(f) adding secondary nucleic acid tags to the at least two secondary aliquots, wherein the secondary nucleic acid tags added to the target molecule-binding agents in the first secondary aliquot are different from the secondary nucleic acid tags added to the target molecule-binding agents in the second secondary aliquot; and
(g) repeating steps (d), (e), and (f) with the at least two secondary aliquots a number of times sufficient to generate a unique series of nucleic acid tags for each exosome, cell or vesicle in the sample.

In some embodiments, the primary nucleic acid tags, the secondary nucleic acid tags and/or subsequent nucleic acid tags described herein above are added by ligation reactions, polymerase extension reactions, and/or chemical syntheses.

In some embodiments, the nucleic acid tags described herein above are added by polymerase extension reaction,
wherein the nucleic acid barcode bound to each target molecule-binding agent is extended with one of the primary nucleic acid tags by contacting the exosomes, cells or vesicles with a strand displacing polymerase and a first DNA hairpin, wherein the first DNA hairpin comprises:
(i) a first oligonucleotide comprising a sequence complementary to the universal round 1 primer sequence, and
(ii) a second oligonucleotide, wherein the second oligonucleotide comprises a third oligonucleotide comprising a primary nucleic acid tag, and a fourth oligonucleotide comprising a sequence complementary to the primary nucleic acid tag;
wherein the third oligonucleotide is located at the 5′ end of the second oligonucleotide, and the fourth oligonucleotide is located at the 3′ end of the second oligonucleotide; and
wherein the first oligonucleotide is fused to the 3′ end of the fourth oligonucleotide.

In some embodiments, each primary nucleic acid tag described herein above comprises a unique well-specific first round barcode sequence at the 5′ end and a universal round 2 primer sequence at the 3′ end.

In some embodiments, the first DNA hairpin described herein above is disabled at the end of each polymerase extension by removal of the first oligonucleotide from the first DNA hairpin using an exonuclease or by treating the first DNA hairpin with an enzyme that remove a unique base present at the junction of the first oligonucleotide and the fourth oligonucleotide.

In some embodiments, the method described herein further comprising adding secondary nucleic acid tags by polymerase extension reaction,
wherein the nucleic acid barcode bound to each target molecule-binding agent is further extended with one of the secondary nucleic acid tags by contacting the exosomes, cells or vesicles with a strand displacing polymerase and a second DNA hairpin wherein the second DNA hairpin comprises:
(i) a fifth oligonucleotide comprising a sequence complementarity to the universal round 2 primer sequence, and
(ii) a sixth oligonucleotide, wherein the sixth oligonucleotide further comprises a seventh oligonucleotide comprising a secondary nucleic acid tag, and an eighth oligonucleotide comprising a sequence encoding a sequence complementary to the secondary nucleic acid tag;
wherein the seventh oligonucleotide is located at the 5′ end of the sixth oligonucleotide, and the eighth oligonucleotide is located at the 3′ end of the sixth oligonucleotide; and
wherein the fifth oligonucleotide is fused to the 3′ end of the eighth oligonucleotide.

In some embodiments, each secondary nucleic acid tag comprises a unique well-specific second round barcode sequence at the 5′ end and a universal round 3 primer sequence at the 3′ end.

In some embodiments, the primary nucleic acid tags described herein above are added by ligation reaction in a first-round splint-ligation reaction, wherein the primary nucleic acid tags are added to the 3′ end of the nucleic acid barcode bound to each target molecule-binding agent by contacting the exosomes, cells or vesicles with a ligase, a first-round oligonucleotide and a first-round splint sequence,
wherein the first-round oligonucleotide comprises a 5′ common region followed by a primary nucleic acid tag terminated by a 3′ universal round 2 sequence; and
wherein the first-round splint sequence comprises a region complementary to the universal round 1 sequence at 3′ end of the nucleic acid barcode bound to each target molecule-binding agent and a region complementary to the 5′ common region of the first-round oligonucleotide.

In some embodiments, the splint-ligation process described above is terminated at the end of the first-round splint-ligation reaction.

In some embodiments, secondary nucleic acid tags are added through a second-round splint-ligation reaction,
wherein the nucleic acid barcode bound to each target molecule-binding agent is further extended with a secondary nucleic acid tag by contacting the exosomes, cells or vesicles with a ligase, a second-round oligonucleotide and a second round splint sequence,
wherein the second round oligonucleotide comprises a 5′ common region followed by a secondary nucleic acid tag terminated by a 3′ universal round 3 sequence or a universal PCR sequence; and
wherein the second round splint sequence comprises a region complimentary to the 3′ universal round 2 sequence of the first-round oligonucleotide, and a region complimentary to the 5′ common region of the second round oligonucleotide.

In some embodiments, the methods described herein above further comprise amplifying and sequencing the nucleic acid barcodes, the UMIs and/or the nucleic acid tags. In some embodiments, split-pool sequencing is used to sequence the barcodes, UMIs and/or nucleic acid tags.

In some embodiments, the target molecules described herein above are proteins, sugar moieties, lipids, and/or polynucleotides.

In some embodiments, the target molecule-binding agents described herein above comprise an antibody, an antibody fragment, a peptide aptamer, lectins, a phage display system or a yeast display system.

In some embodiments, the nucleic acid barcode described herein above is a DNA-barcode.

In some embodiments, the detecting method described herein above is at a single cell or a single exosome or a single vesicle level.

In some embodiments, each of the at least two primary aliquots described herein above consists of a single cell, a single exosome, or a single vesicle.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1. Overview of an exemplary ligation reaction. A barcode is added to the 3′ end of an antibody barcode through a splint ligation process (FIG. 1, steps (a)-(b)).

FIG. 2. Overview of polymerase extension reaction (PER). Barcoding at each “split” step is performed using Polymerase Extension Reaction (PER). Hairpins are designed with a 3′ poly dA tail to prevent priming and extension on the 3′ end of the hairpin. The barcode sequence in green to be appended to the antibody is well-specific and comprises a sequence that allows for subsequent second round extension or PCR amplification depending on the barcoding capacity required for the experiment. Notably, the hairpin can also possess a modified base in the first paired position (denoted by the star). This allows for enzymatic digestion of the hairpin after the reaction to minimize crosstalk and mis-extension in the subsequent “split” extension/PCR steps. Modifications may include but are not limited to, uracil, inosine, or 5′ hairpin phosphorylation (not pictured). FIG. 2, step (a): Antibodies bound to exosomes are conjugated with oligos that possess an antigen specific barcode and UMI, as well as a 3′ terminal landing sequence. They are introduced in solution with a barcoding hairpin, a strand displacing polymerase, appropriate dNTPs, and supplemented with enzyme buffer. The reaction is allowed to proceed isothermally at a temperature appropriate to the sample and the polymerase. FIG. 2, step (b): The 3′ terminal landing sequence anneals on the exposed complementary 3′ overhang on the hairpin. FIG. 2, step (c): The strand displacing polymerase extends the oligo, appending to it the appropriate barcode sequence before encountering a stop signal prompting the reaction to terminate. FIG. 2, step (d): Branch migration allows the newly extended oligo on the antibody to spontaneous dissociate from the hairpin. FIG. 2, step (e): The hairpin, newly re-annealed, is recycled to extend other unextended oligos in solution. FIG. 2, step (f): After the reaction is completed the appropriate enzyme is introduced into the reaction to cleave the landing pad from the hairpin to prevent inappropriate extension of unextended oligos from the current round in subsequent rounds.

FIG. 3. Design of DNA oligonucleotide and barcoding hairpins. FIG. 3, step (a): Antibody barcodes are conjugated such that each antibody will have an antibody-specific barcode that encode the identity of the corresponding antigen to which the antibody binds as well as a UMI. All oligos have the same “universal round 1” sequence appended to their 3′ end to facilitate extension by PER. FIG. 3, step (b): The first round hairpin has a 3′ poly dA tail to prevent priming and extension on the 3′ end of the hairpin. Each hairpin also contains a unique well-specific first round barcode as well as a universal sequence common to all hairpins in the same round for use during the next round of extension. The sequence that gets extended by the strand displacing polymerase is comprised of As, Cs, and Ts. The black circle shown in the diagram represents a C within the hairpin template which would require the addition a G but the reactions are setup devoid of dGTP and as such the polymerase stalls upon the need to add this base and does not extend the oligonucleotide any further. FIG. 3, step (c): The second round hairpin is similar in structure and function to first except it contains unique sequences which enable its use for extension by only DNA oligonucleotides that were correctly extended during the first round of PER. Similar to the first round, each hairpin that is provided to a well labels all molecules with a unique well-specific second round barcode as well as a universal sequence common to all hairpins in the same round for use during additional rounds of PER as for PCR to generate the final product that is ready for DNA sequencing, with e.g., split-pool sequencing.

Note that here dashed species represent barcode regions that vary from antibody to antibody or between hairpin barcodes. Solid lines represent sequences that are universal among all oligonucleotides of the same type. Furthermore, the modification from the first and second round extensions (denoted by star and hexagon) must differ to prevent premature degradation of hairpins from enzyme carryover of the first round reaction. Alternatively, a method can be used to disable the enzymes that excise the unconventional base after they have performed their function to enable the same approach to disabling the hairpin to be used for all rounds. While not depicted, other methods of selective hairpin destruction such as, but not limited to, lambda exonuclease based digestion of the hairpin via the addition of 5′ phosphate groups to all hairpins, can also be used. (see, e.g., J. Y. Kishi, et al., 2018. Programmable autonomous synthesis of single-stranded DNA. Nature Chemistry, pp. 155-164, the contents of which are hereby incorporated herein by reference).

FIG. 4. Represents an exemplary polymerase extension reaction (PER) comprising two rounds of barcode extension and enzymatic digestion of hairpins following each round, and a third round of barcode PCR.

FIG. 5A-5D. Platform for performing single-cell immune profiling: FIG. 5A: Antibody with a conjugated DNA oligo. Each oligo contains an antibody specific barcode and a UMI. FIG. 5B: Antibodies bind specifically to their cellular targets. FIG. 5C: During split-pool sequencing, all of the oligos associated with a given cell are identically labeled, yet no two cells apply the same tag to their oligos. FIG. 5D: During analysis, all oligos associated with a given cell are identified via their tag, and the composition and quantity of all bound antibodies is determined by analyzing the BC and UMI, respectively. For simplicity, only antibodies binding to cell surface proteins are shown.

FIGS. 6A and 6B. Overview of a single exosome sequencing (SESeq) approach. FIG. 6A: A million unique DNA barcoded gel beads are mixed with isolated exosomes along with the required enzymes for reverse transcription using microfluidics. Oil is then used to partition the sample, isolating a single bead with a single exosome. Each exosome is then reverse transcribed and all RNAs from a single exosome are tagged with a common DNA barcode. FIG. 6B: Upon sequencing the RNAs belonging to each exosome can be determined by using the unique DNA barcode added to each transcript during sample preparation. This allows to know all the RNAs within in each exosome.

FIG. 7. The computational algorithm used in the methods of the disclosure is robust to sparse and noisy data. Used algorithms include single cell clustering algorithms, such as the metaVIPER. Box plots show how well one (box) or more cells correlate (Pearson's r) with bulk RNA-seq data. The light grey plots show the correlation when using gene expression data. The correlation between two cells is approximately 0.95, while using gene expression about 10-fold more cells are required for a similar correlation.

FIG. 8. Overview of exemplary methods of the disclosure used in clinical diagnostics. A blood sample is first collected from a cancer patient undergoing chemotherapy, exosomes are isolated, SESeq is performed and used to monitor tumor response to therapy and to determine potential deleterious side-effects from chemotherapy.

FIGS. 9A and 9B. Schematic of protein split-pool sequencing (PSP-seq) experimental design. FIG. 9A: Cells are stained collectively with a curated panel of DNA-barcoded antibodies. Stained cells go through a series of split and pool steps where a unique well barcode is appended to the 3′ end of the DNA barcode. A single cell (orange) has been highlighted for clarity as it travels through the barcoding process. FIG. 9 B: Schematic of the design of the DNA barcode. First round (FIG. 9B step (i)) and second round (FIG. 9B step (ii)) well barcodes are appended to antibody-conjugated DNA barcode conjugated to the antibody through two initial rounds of ligation. In ligation steps, a splint primer is used to bring the two oligos in proximity to each other and to allow T4 DNA ligase to perform DNA ligation. Third round barcodes ((FIG. 9, step (iii)) are added through PCR before libraries are prepared for sequencing.

FIG. 10. Experimental schematic for two cell line mixing PSP-seq experiment with doped in HT2- and HT3-labeled Jurkat cells to benchmark off-target noise and errors introduced during sample preparation and amplification.

FIG. 11A—11E. Benchmarking of PSP-seq technology. FIG. 11A: HEK293T and Jurkat cells were stained separately with an antibody cocktail containing the antibodies listed. Results from bulk sequencing of DNA-barcoded antibodies bound to stained cells are shown here. FIG. 11B: In a two-cell line mixing experiment where HEK293T and Jurkat cells are used, CD56 and CD4 can be used, respectively, to distinguish the identity of the cell type in question. HT2 control cells as expected did not stain for either CD56 or CD4. Out of a total of 474 sample cells, only 12 were identified as collisions (2.5%). FIG. 11C: Jurkat cells were co-stained with an antibody mix containing anti-CD4 DNA-barcoded antibody and anti-CD4-FITC. Cells were sorted by FACS for CD4 expression level and high, medium, and low populations were obtained. Sorted cells from the three populations were then uniquely labeled with different hashtag antibodies before collectively passing through PSP-seq to quantify CD4 expression through sequencing of anti-CD4 DNA barcoded antibodies. FIG. 11D: CD4 expression levels are shown as determined by CD4-FITC antibody levels. FIG. 11E: CD4 expression levels are shown as determined by CLR-transformed CD4 antibody barcode counts.

FIG. 12A-12D. Benchmarked statistics for two-cell-line experiment. FIG. 12A: Unique molecular identifiers (UMIs) detected per cell, ordered by the number of UMIs detected. Cells with at least 100 unique UMIs detected were kept for further processing. FIG. 12B: Average coverage per cell was calculated by taking total reads detected per cell divided by the number of unique UMIs. FIG. 12C: Top two components of principal component analysis of two-cell-line experiment. Three populations of known cells—Jurkat, HEK293T, HT2-stained control Jurkat cells—are distinct. A small fraction of cells show unclear characteristics, expressing both CD4 and CD56, resulting in ambiguous cell-type calling and being labeled as “collisions”; these cells are expected to occur at low frequency and hypothesized to be generated due to two cells receiving the same trio of well barcodes. FIG. 12D: Decomposition of the top two principal components. Principal component 1 features cell markers that distinguish the two main cell types (Jurkat, HEK293T). Principal component 2 is heavily dominated by HT2, serving to separate the control HT2 cells from the other two stained populations.

FIG. 13A-13B. Two dimensional TSNE visualization of pooled patient cell samples. FIG. 13A: Individual cells identified in split pool demultiplexing from 10 pooled patient samples. All cells from a given patient are colored the same. FIG. 13B: Two dimensional TSNE of pooled patient samples, color-coded by CLR-transformed protein scores for all 29 proteins sampled.

FIG. 14. Patient cells in 2D TSNE-space (as in FIG. 13B). Single panels are separated by patient ID as determined by hashtag labeling.

FIG. 15A-15D. PSP-seq CLR-transformed antibody scores and flow cytometry from patient 1 bone marrow sample. FIG. 15A: Cells from patient 1 are gated for CD45+ expression. Further stratification of B-cells in this population shows that CD19+CD3− gated B-cells show B-cells that have moderate CD8 expression. FIG. 15B: CD20+ gated B-cells show a pathological expansion of the kappa light chain population. FIG. 15C: Flow cytometry data for the same sample. Monocytes, granulocytes and lymphocytes are identified; lymphocytes are gated for downstream analysis. Population proportion of CD19+CD3− as identified in flow cytometry similar to PSP-seq (85% and 84%, respectively, from FIG. 15A). FIG. 15D: Bright CD20 expression gated to capture B-cell population. Imbalance in kappa to lambda ratio shows 85% of the B cell population is kappa+, concordant with clonal expansion identified in PSP-seq.

FIG. 16A-16B. TSNE colormap of patient samples for (FIG. 16A) patient 1 and (FIG. 16B) patient 9.

FIG. 17A-17D. PSP-seq CLR-transformed antibody scores and flow cytometry from patient 9 bone marrow sample. FIG. 17A: Cells from patient 9 are gated for CD45+ expression. Gating for T-cells using CD3+CD19− reveals an expanded CD4+ T-cell population. FIG. 17B: Large proportion of cells revealed CD4+CD25+ phenotype. FIG. 17C: Population of CD3+ T-cells showed elevated PD-1 and CTLA-4. FIG. 17D: Flow cytometry of the same sample. Cells were gated for lymphocytes, CD45+. Comparison between PSP-seq and flow cytometry showed high concordance in gated populations. Population proportions between PSP-seq and flow cytometry are consistent throughout.

### DETAILED DESCRIPTION

The present disclosure is directed, at least in part, to methods and systems for accurately quantifying the levels of multiple, e.g., over a hundred or more, target molecules, e.g., proteins, in, or on the surface of, single entities, including single exosomes, single cells or single vesicles. In some embodiments, the disclosure provides methods for high throughput detection of a plurality of target molecules within pooled single entities, including single exosomes, single cells or single vesicles using DNA-barcoded antibodies (DBAs), split-pool sequencing, and/or unique molecule identifiers (UMIs).

The methods and systems of the disclosure require no specialized equipment to scale to hundreds of protein targets, thereby providing cost-effective, modular and easy-to-use quantitative single cell protein profiling platform.

This platform provides methods for use in, e.g., preclinical and clinical settings for diagnostic and therapeutic applications, using DBAs and split-pool sequencing to provide cost effective, modular and simple to use quantitative protein profiling of a single entity, e.g., a single exosome, a single cell, or a single vesicle.

In some embodiments, the methods and systems of the disclosure can be applied to early stage discovery pipelines to identify pathways of interest (e.g., in a preclinical setting) and in clinical settings for diagnosis, monitoring, or therapy of various diseases or disorders, such as immune diseases and disorders, e.g., cancer, autoimmune disease, and inflammatory disease.

In some embodiments, the methods and systems of the disclosure can be applied to identify which patients with a disease or disorder, e.g., cancer, autoimmune disease or inflammatory disease, are more or less likely to respond to any of a diverse set of therapeutics, e.g., immune regulators (such as, for example, an anti-PD1 antibody). Furthermore, in some embodiments, the methods and systems of the disclosure can be used to monitor a response to a particular therapeutic.

Furthermore, the methods of the disclosure can be used for quantifying the abundance and state (e.g. phosphorylation state) of hundreds of proteins at the level of a single entity, e.g., cell, vesicle or exosome, enables comprehensive phenotyping within a variety of disease states, and have translational applications in early stage discovery pipelines or in guiding clinical decision making.

The methods of the disclosure are superior to existing technologies which require specialized equipment and lack the ability to scale to hundreds of protein targets (e.g., mass cytometry (CyTOF), immunofluorescence microscopy, flow cytometry).

Because of its potential to simultaneously monitor multiple tissues, methods of the disclosure are powerful tools for studying off-target drug response studies which may be of interest in, for example, chemotherapies, where both on-target effects at a tumor and off-target effects at healthy tissue can be simultaneously monitored.

Recent work in bioinformatics, which allows an understanding of genetic interactions and network dynamics of gene signaling, expands the impact of this technology by allowing for educated inferences about the dynamics of intracellular activity by protein signaling manifested on the cell surface.

Thus, the methods and systems of the disclosure provide a cost-effective, modular, and easy-to-use quantitative single entity (e.g., cell, exosome, or vesicle) protein profiling platform. These methods utilize DNA-barcoded antibodies and, optionally, unique molecule identifiers (UMIs) in combination with split-pool sequencing to improve information content and to reduce cost. The methods enable multiplexed and highly scalable detection of proteins at the single entity level. These methods and systems do not require in-house specialized equipment or training and can be applied in early stage discovery pipelines to identify pathways of interest and in clinical settings to diagnose and monitor treatment of patients, and to determine and/or predict which patients are more or less likely to respond to specific therapeutics, e.g., immune regulators, such as, for example, anti-PD1 antibodies, for the treatment of cancer, autoimmune disease, or inflammatory disease.

### Definitions

As used herein and in the appended claims, the singular forms “a”, “an”, and “the” include plural reference unless the context clearly dictates otherwise. Thus, for example, reference to a “protein” is a reference to one or more proteins and equivalents thereof known to those skilled in the art, and so forth.

As used herein, the term “about” means plus or minus 20% of the numerical value of the number with which it is being used. Therefore, about 50% means in the range of 40%-60%.

The transitional term “comprising”, which is synonymous with “including,” “containing,” or “characterized by,” is inclusive or open-ended and does not exclude additional, unrecited elements or method steps. See MPEP 2111.03.

As used herein, the term “target molecule-binding agent” as used herein refers to any naturally occurring or synthetic biological or chemical molecule which specifically binds an identified target molecule. The binding can be covalent or non-covalent, i.e., conjugated or by any known means taking into account the nature of the target molecule-binding agent and its respective target. In some embodiments, a target molecule-binding agent is a protein, e.g., an antibody, that specifically binds to a target protein. In some embodiments, the target molecule-binding agent comprises an antibody, an antibody fragment, or a peptide aptamer.

The term “antibody”, as used herein, broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Examples antibody fragments include, but are not limited to, Fab, Fab′, F(ab′)2, Fv, immunologically functional immunoglobulin fragments, heavy chain, light chain, and single-chain antibodies. Such mutant, variant, or derivative antibody formats are known in the art. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY), class (e.g., IgG 1, IgG2, IgG 3, IgG4, IgA1 and IgA2) or subclass.

The term “barcode” as used herein, refers to any unique, non-naturally occurring, nucleic acid sequence that may be used to identify the originating source of a molecule. Barcoding may be performed based on any of the compositions or methods disclosed in patent publication WO 2014047561 A1, “Compositions and methods for labeling of agents,” incorporated herein in its entirety. Additionally, other barcoding designs and tools have been described (see e.g., Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94); Rosenberg et al. (2018) Science, 360; 176-182; Quinodoz, et al. (2018) Cell, 174, 744-757, the contents of each of which are hereby incorporated herein by reference. In some embodiments, the nucleic acid barcode is a DNA barcode. In some embodiments, a barcode, e.g., a DNA barcode, is associated with a protein, e.g., an antibody.

As used herein, the term “oligonucleotide” refers to a nucleic acid such as deoxyribonucleic acid (DNA), ribonucleic acid (RNA), or DNA/RNA hybrids and includes analogs of either DNA or RNA made from nucleotide analogs known in the art (see, e.g. U.S. Patent or Patent Application Publications: U.S. Pat. Nos. 7,399,845, 7,741,457, 8,022,193, 7,569,686, 7,335,765, 7,314,923, 7,335,765, and 7,816,333, US 20110009471, the entire contents of each of which are incorporated herein by reference). Oligonucleotides may be single-stranded (such as sense or antisense oligonucleotides), double-stranded, or partially single-stranded and partially double-stranded.

As used herein, “sample” includes a specimen or culture obtained from any source which contains a cell, exosome, or vesicle. Biological samples can be obtained from blood (including any blood product, such as whole blood, plasma, serum, or specific types of cells of the blood), a blood fraction containing peripheral blood mononuclear cells, cerebrospinal fluid (CSF), lymph, urine, saliva, semen, sweat, sputum, lacrimal fluid, synovial fluid, cerebrospinal fluid, feces, mucous, vaginal fluid, and spinal fluid. Biological samples also include tissue samples, such as biopsy tissues or pathological tissues that have previously been fixed (e.g., formaline snap frozen, cytological processing, etc.). Methods or obtaining tissue biopsies and body fluids from mammals are well known in the art.

Methods of the Disclosure

The present disclosure is directed, at least in part, to methods and systems for accurately quantifying the levels of multiple, e.g., over a hundred or more, target molecules, e.g., proteins, in, or on the surface of, single entities, including single exosomes, single cells or single vesicles. In some embodiments, the disclosure provides methods for high throughput detection of a plurality of target molecules in or on pooled single entities, e.g., single exosomes, single cells or single vesicles using DNA-barcoded antibodies (DBAs) and split-pool sequencing.

Accordingly, in some aspects, the present disclosure provides methods for detecting a plurality of target molecules, e.g., proteins, in, or on the surface of exosomes, cells or vesicles. In some embodiments exosomes, cells or vesicles in a sample are placed in contact with a plurality of target molecule-binding agents, e.g., antibodies, wherein each target molecule-binding agent, e.g., antibody, comprises a nucleic acid barcode and optionally a unique molecular identifier (UMI), and wherein target molecule-binding agents, e.g., antibodies, that are specific to an identical target molecule share an identical nucleic acid barcode.

In some embodiments, a vesicle, a microvesicle, a membrane particle, and/or an apoptotic bleb. In some embodiments, a vesicle is a biological organelles or complexes. In some embodiments, the biological organelle or complex is mitochondria, lysosome, and/or Golgi body. In other embodiments, a vesicle is a synaptosome, a nanoparticle, and/or DNA origami. In some embodiments, the vesicle is are natural or synthetic.

The cells used in the methods of the disclosure can be of any cell type. In some embodiments, the cells are immune cells. In other embodiments, the cells are cancer cells. In other embodiments, the cells are endothelial cells, stem cells, bone cells, blood cells, muscle cells, cardiomyocytes, fat cells, skin cells, nerve cells, brain cells, pancreatic cells, sperm cells, egg cells, or any other cell type.

In some embodiments, each target molecule-binding agent, e.g., antibody, further comprises a universal primer sequence at the 3′ end for a first round extension, e.g., a universal round 1 primer sequence. In some embodiments, the universal primer sequence comprises about 9 to 20 nucleotides. In some embodiments, the universal primer sequence is a 10-mer. In some embodiments, the universal primer sequence is a 15-mer.

In some embodiments, the methods described herein further comprises:
(b) dividing the exosomes, cells or vesicles into at least two primary aliquots, the at least two primary aliquots comprising at least a first primary aliquot and a second primary aliquot;
(c) adding primary nucleic acid tags to the target molecule-binding agents in the at least two primary aliquots, wherein the primary nucleic acid tags added to the target molecule-binding agents in any one of the at least two primary aliquots are different from the primary nucleic acid tags added to the target molecule-binding agents in any one of the other primary aliquots;
(d) combining the at least two primary aliquots;
(e) dividing the combined primary aliquots into at least two secondary aliquots, the at least two secondary aliquots comprising a first secondary aliquot and a second secondary aliquot; and
(f) adding secondary nucleic acid tags to the at least two secondary aliquots, wherein the secondary nucleic acid tags added to the target molecule-binding agents in the first secondary aliquot are different from the secondary nucleic acid tags added to the target molecule-binding agents in the second secondary aliquot.

In some embodiments, the method further comprises step (g), which comprises repeating steps (d), (e), and (f) with the at least two secondary aliquots a number of times sufficient to generate a unique series of nucleic acid tags for each exosome, cell or vesicle in the sample.

In some embodiments, the methods described above further comprise amplifying and sequencing the nucleic acid barcodes, the UMIs and the nucleic acid tags. In some embodiments, next generation sequencing is used to sequence the barcodes, UMIs and/or nucleic acid tags. In some embodiments, split-pool sequencing is used to sequence the barcodes, UMIs and/or nucleic acid tags.

In some embodiments, the target molecules are proteins, sugar moieties, lipids, and/or polynucleotides.

In some embodiments, the detecting is at a single cell or a single exosome, cell or vesicle level. In some embodiments, each of the at least two primary aliquots consists of a single cell, a single exosome, or a single vesicle. In some embodiments, the at least two primary aliquots is about 4, 5, 6, 7, 8, 9, 10, 20, 30, 40, 50, 60, 70, 80, 90, 100, 200, 300, 400, 500, 600, 700, 800, 900 or 1000 or more aliquots.

In some embodiments, the nucleic acid tags are added by ligation reactions, polymerase extension reactions, and/or chemical syntheses.

Ligation Reactions

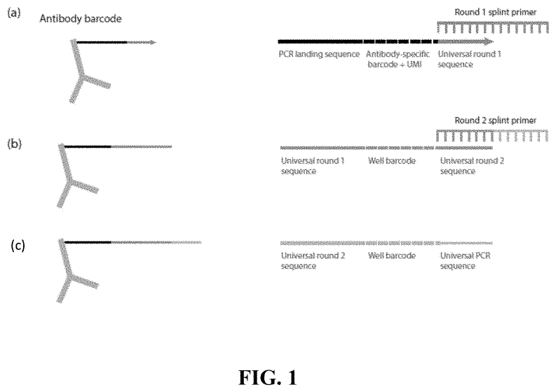

In some embodiments, the primary nucleic acid tags and/or the secondary nucleic acid tags and/or subsequent nucleic acid tags are added by ligation reaction(s). Because DNA barcodes are conjugated on their 5′ ends to their respective target molecule-binding agents (e.g., antibodies), barcode addition must occur on the 3′ end of the antibody-bound DNA barcode. In order to use split-pool sequencing (Rosenberg et al. (2018) Science, 360; 176-182, the contents of which are incorporated herein by reference), the ligation reaction was adapted to accommodate 3′ end ligations (see, e.g., FIG. 1A-1C).

In such a ligation method, one or more barcodes (i.e., nucleic acid tags) are added to the 3′ end of the target molecule-binding agent barcode (e.g., antibody barcode) through at least one splint-ligation reaction.

As a non-limiting example, FIG. 1A-1C illustrate an exemplary ligation reaction method of the disclosure using an antibody. In some embodiments, a pool of exosomes, cells or vesicles to which various DNA-conjugated antibodies have bound is split into multiple wells. In some embodiments, a first-round oligonucleotide and a first-round splint sequence are added to each well containing a single exosome, a single cell or a single vesicle, bound to the target molecule-binding agent barcode, and a first-round splint ligation reaction is carried out. In some embodiments, the target molecule-binding agent barcode further comprises a universal round 1 sequence.

In some embodiments, the first-round oligonucleotide comprises a 5′ common region, e.g., a universal round 1 sequence, followed by a unique barcode sequence (primary nucleic acid tag) terminated by a 3′ region common to all first round oligonucleotides (e.g., a universal round 2 sequence). In some embodiments, the 5′ common region and the 3′ universal round 2 sequence are about 15 bases in length.

In some embodiments, the first-round splint sequence comprises a region complementary to the universal round 1 sequence at 3′ end of the target molecule-binding agent barcode and a region complementary to the 5′ end of the first-round oligonucleotide in order to facilitate double stranded DNA ligation (see FIG. 1, steps (a)-(b)).

In some embodiments, at the end of the first-round splint ligation reaction, a blocking strand is added to prevent inappropriate subsequent ligations. In some embodiments, the first-round blocking strand sequence is complementary to the first-round splint sequence.

In some embodiments, following the first-round splint ligation reaction, a second round splint ligation process is carried out. In some embodiments, the exosomes, cells or vesicles are re-pooled and split into second round wells. In some embodiments, a second round oligonucleotide and a second round splint sequence are then added to each well.

In some embodiments, the second round oligonucleotide comprises a common 5′ region (e.g., a universal round 2 sequence), a second round barcode (secondary nucleic acid tag), and a common 3′ region (e.g., a universal round 3 sequence or a universal PCR sequence). In some embodiments, the 5′ common region and the 3′ common region are about 15-bases in length.

In some embodiments, the second round splint sequence comprises a region complementary to the 3′ universal round 2 sequence of the first-round oligonucleotide, as well as a region complementary to the 5′ common region of the second round oligonucleotide (see FIG. 1 step (c)).

In some embodiments, after ligation, a second round blocking strand that is complementary to the second round splint sequence is added.

One or more subsequent ligation reactions can be repeated for as many rounds as necessary to generate necessary barcode diversity.

Before final addition by PCR, the reaction can be terminated by the addition of a buffer containing EDTA. The final round barcode can be added by PCR (see FIG. 1, step (c)).

Polymerase Extension Reactions

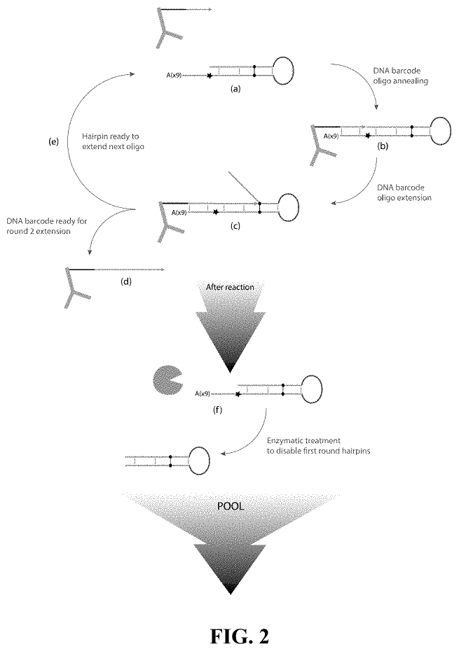

In some embodiments, the primary nucleic acid tags and/or the secondary nucleic acid tags and/or subsequent nucleic acid tags are added by polymerase extension reactions (PER). PER is a strand-displacing DNA polymerase-based barcoding approach (generally described in, for example, Kishi et al., Nature Chemistry, Nov. 6, 2017, DOI: 10.1038, the contents of which are hereby incorporated herein by reference). In PER, a DNA oligonucleotide bound to a target of interest, e.g., antibody, is extended by a strand displacing polymerase by providing a DNA hairpin that has complementarity to the 3′ end of the oligonucleotide conjugated to targets of interest (FIG. 2, steps (a)-(b)).

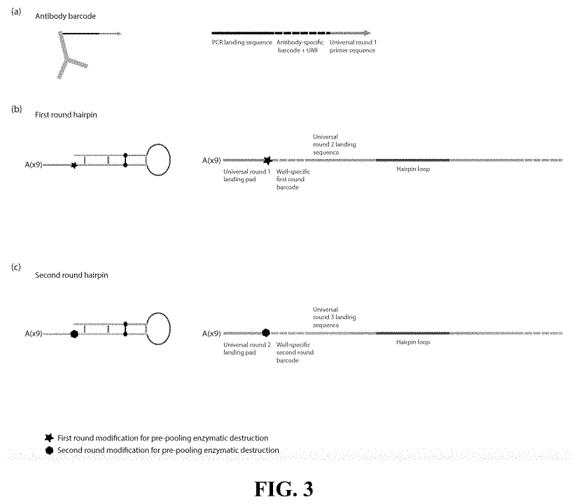

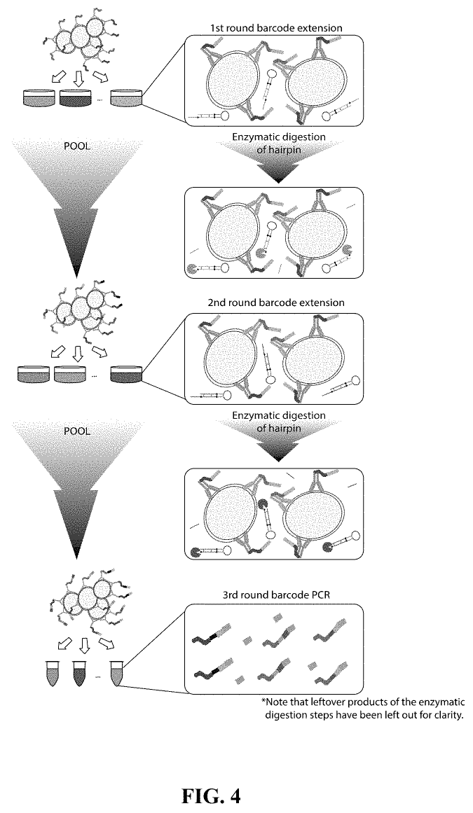

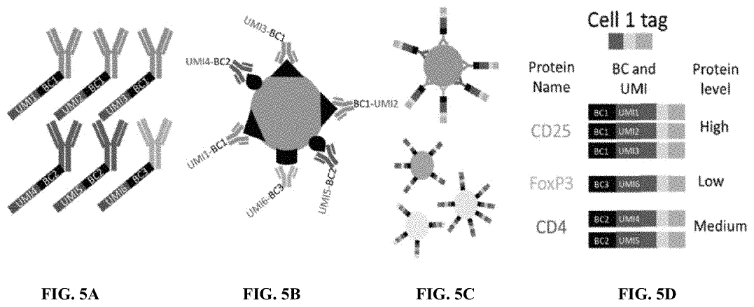

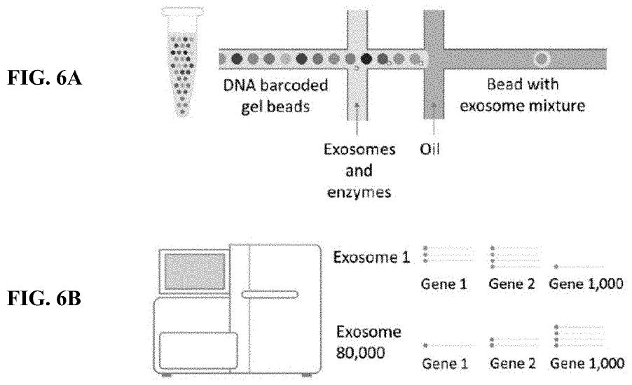

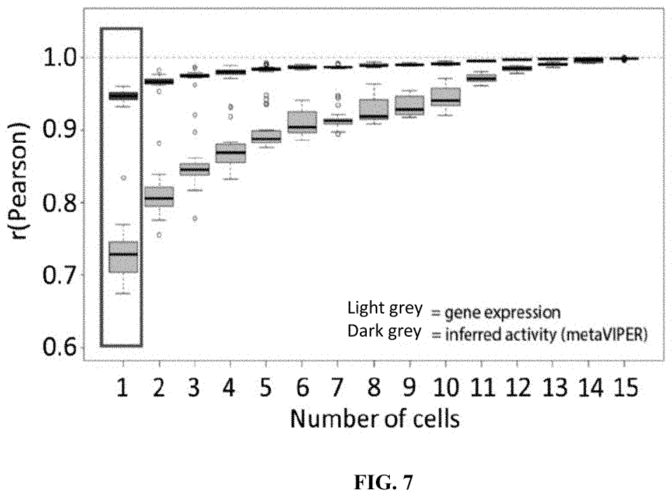

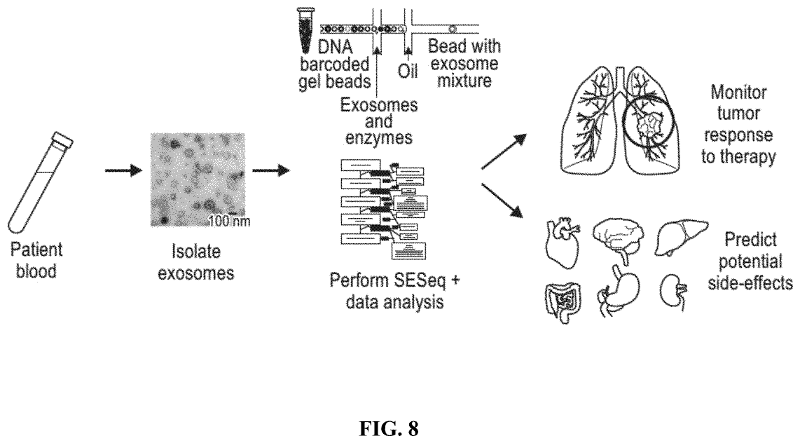

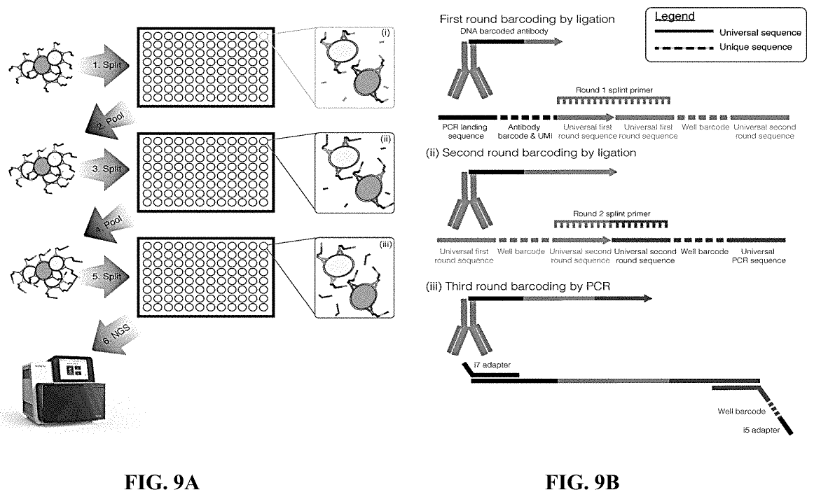

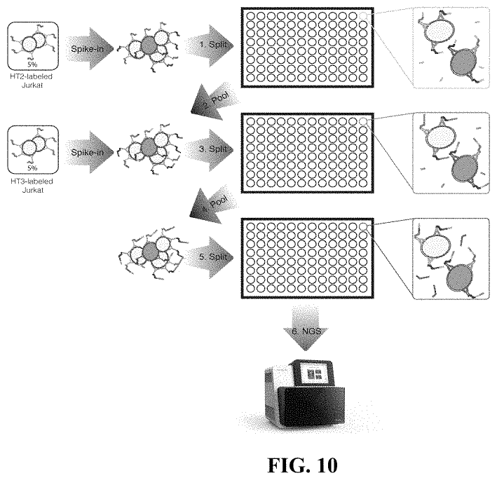

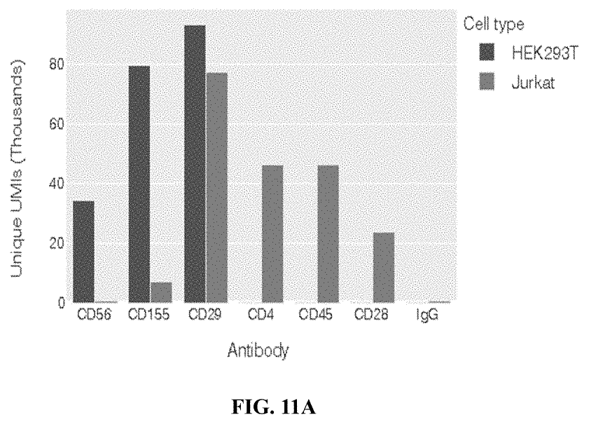

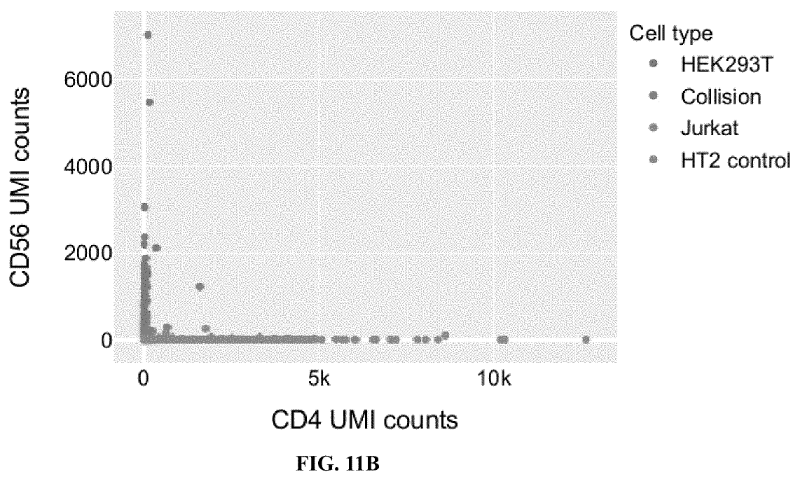

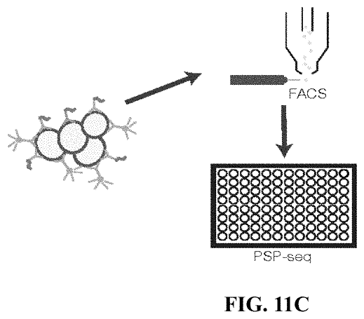

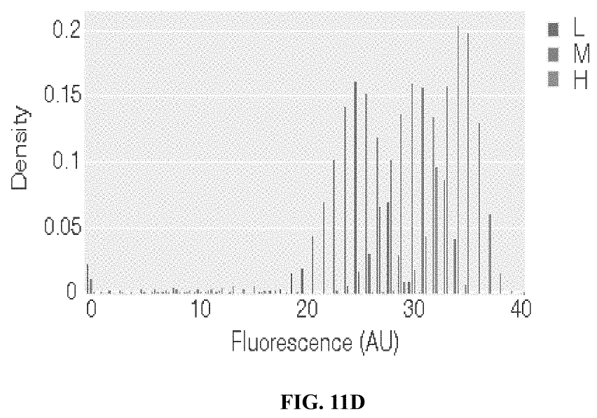

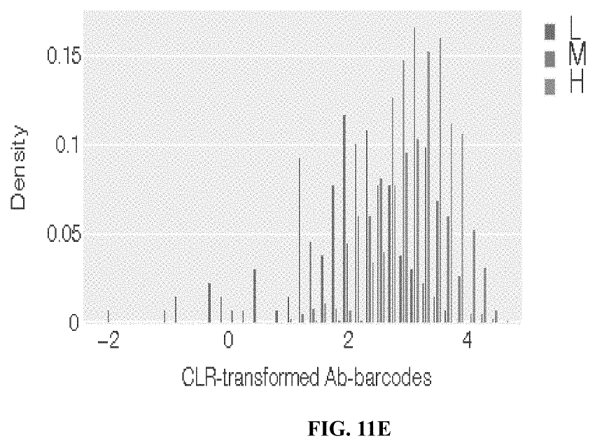

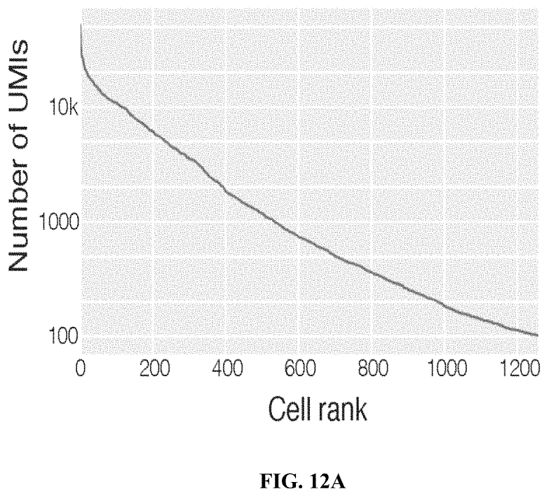

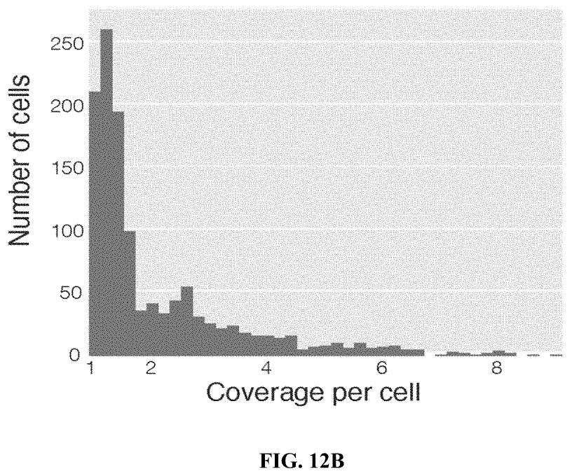

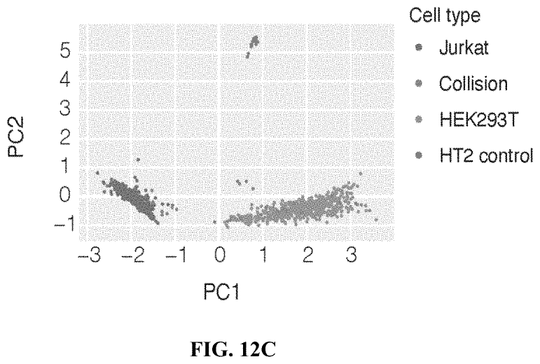

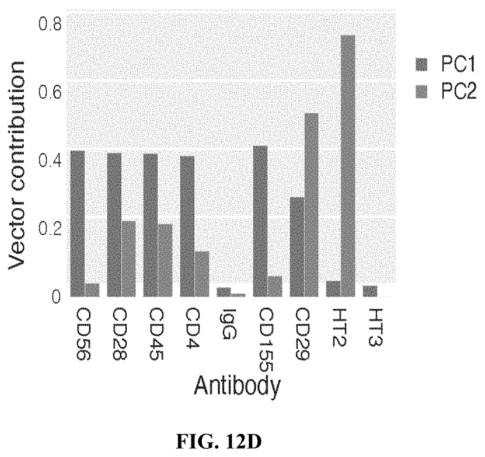

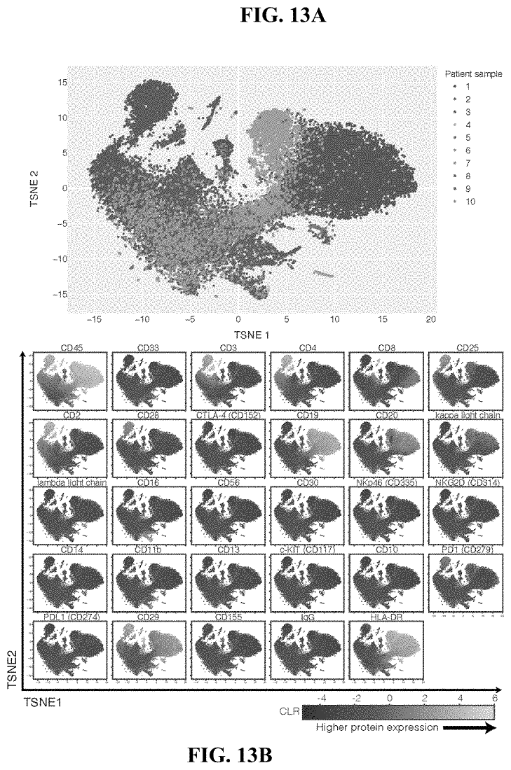

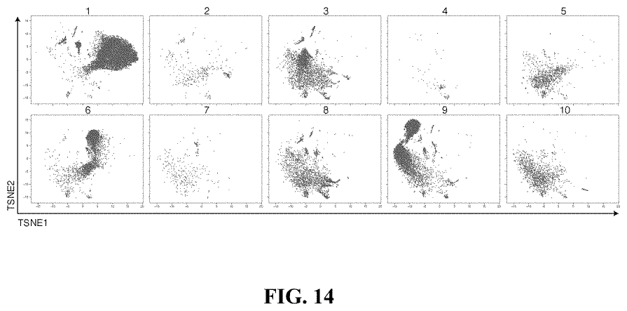

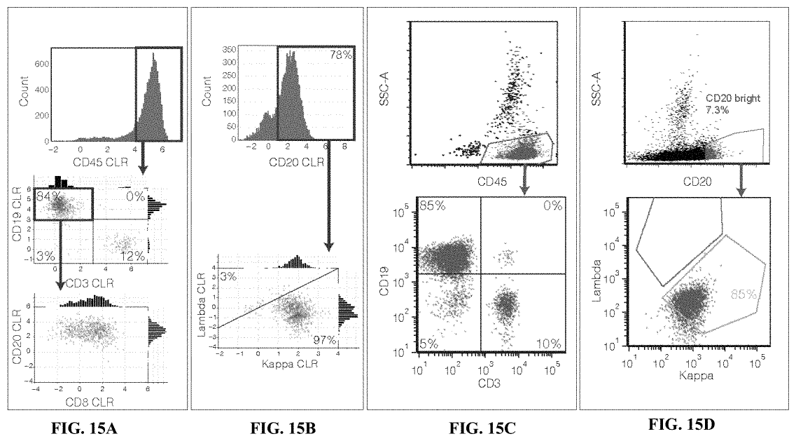

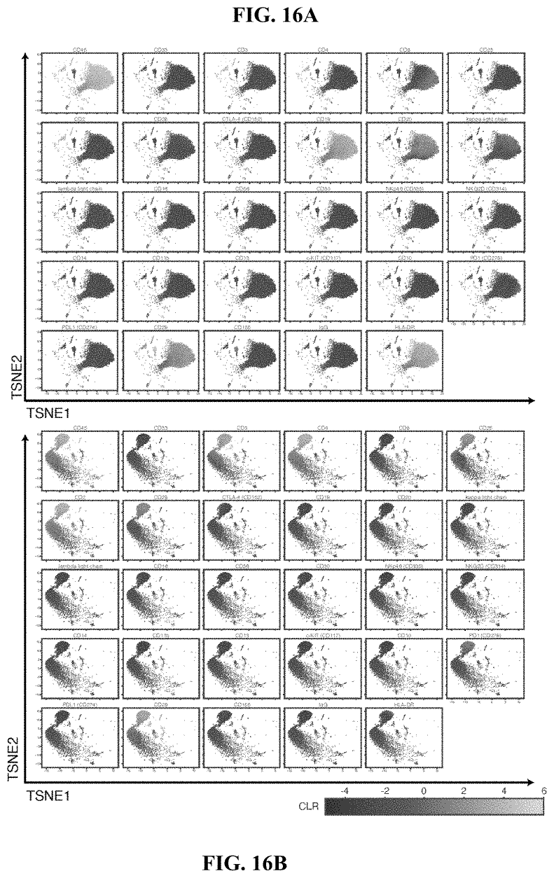

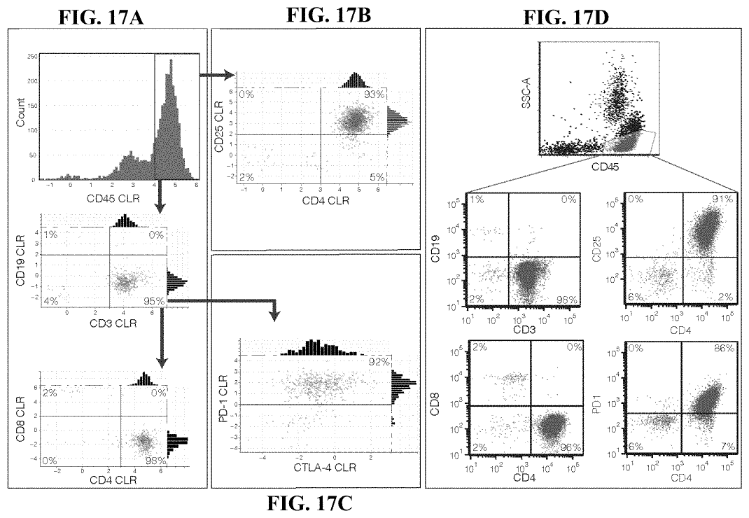

As a non-limiting example, FIGS. 2-4 illustrate the PER methods of the disclosure using an antibody. However, any target of interest that can have a DNA oligonucleotide associated with it can be used in the methods of the disclosure, including, but not limited to, a cell, organelle, microvesicle, DARPin, fynomer, nanobody, or monobody.

In some embodiments, during the PER reaction, the oligonucleotide associated with the target of interest, e.g., the antibody, is extended, and during this extension it obtains a unique “first round barcode” or “primary nucleic acid” (FIG. 2, step (c)). At the reaction temperatures used to perform PER, the complex between the hairpin and the DNA oligonucleotide is susceptible to branch migration, a process in which the displaced hairpin flap competes and causes the extended oligonucleotide to disassociate from the hairpin, thus freeing the hairpin so that it can be used in subsequent rounds of PER (FIG. 2, step (e)). As the outcome of PER is the addition of a unique sequence to the DNA oligonucleotide attached to a target of interest (FIG. 2, step (d)), this principle can be used as a method of barcoding, as an alternative to a ligation-based approach.

In some embodiments, to enable PER for use in for antibody barcoding, a pool of exosomes, cells or vesicles to which various DNA-conjugated antibodies have bound is split into multiple wells. Each oligonucleotide in the same well is given a first round hairpin that appends a unique “well-specific first round barcode” sequence along with a common “universal round 2 landing sequence” to all the oligonucleotides in the well. After this first round of labeling, the hairpins within each of the wells are disabled using, for example, an exonuclease or by treating the hairpins with enzymes that will remove a unique base present within each of the hairpin separating the landing pad sequence from the rest of the hairpin (FIG. 2F). By disabling the first round hairpins, inappropriate hairpin priming between wells when they are pooled in subsequent rounds is prevented.

Following the first round of barcoding and hairpin destruction, all wells are pooled together, mixed, and then aliquoted into a fresh set of wells. The same process of hairpin-based extension is then repeated using a set of second round hairpins, with the process repeated as many times as desired to give the amount of barcode diversity needed for the user's purposes.

After the final round of PER, the universal landing sequence that is added can be used as a PCR binding site to add further library diversity and also prepare the library for next generation sequencing.

Accordingly, in some embodiments, the present disclosure provides methods wherein the nucleic acid barcode bound to each target molecule-binding agent, e.g., antibody, is extended with one of the primary nucleic acid tags by contacting the exosomes, cells or vesicles with a strand displacing polymerase and a first DNA hairpin.

In some embodiments, the first DNA hairpin comprises
(i) a first oligonucleotide comprising a sequence complementary to a universal round 1 primer sequence, and
(ii) a second oligonucleotide, wherein the second oligonucleotide comprises a third oligonucleotide comprising a sequence comprising a primary nucleic acid tag, and a fourth oligonucleotide comprising a sequence complementary to the primary nucleic acid tag;
wherein the third oligonucleotide is located at the 5′ end of the second oligonucleotide, and the fourth oligonucleotide is located at the 3′ end of the second oligonucleotide; and
wherein the first oligonucleotide is fused to the 3′ end of the fourth oligonucleotide.

In some embodiments, each primary nucleic acid tag comprises a unique well-specific first round barcode sequence at the 5′ end and a universal round 2 primer sequence at the 3′ end.

In some embodiments, the second oligonucleotide further comprises a connecting sequence between the third and fourth oligonucleotides. In some embodiments, the connecting sequence comprises the nucleic acid sequence GGGCCTTTTGGCCC (SEQ ID NO: 1).

In some embodiments, the first DNA hairpin further comprises a polyA sequence at the 3′ end. In some embodiments, the polyA nucleic acid sequence is AAAAAAAA.

An exemplary hairpin DNA sequence that can be used in the methods of the disclosure is as follows:

(SEQ ID NO: 2)
ATACTTATTCTTACTAAATTCAGGGCCTTTTGGCCCTGAATTTAGTAAG
AATA AGTA/ideoxyU/TTGCTAGGACAAAAAAAA,

wherein ATACTTATTCTT (SEQ ID NO: 3), as a third oligonucleotide as mentioned above, is the well index,
TTGCTAGGAC (SEQ ID NO: 4) is the landing pad sequence for the current round, which is complementary to the universal round 1 primer,
ACTAAATTCA (SEQ ID NO: 5) is the common landing pad sequence for the next round,
GGGCCTTTTGGCCC (SEQ ID NO: 1) serves as the connecting sequence, and
TGAATTTAGTAAGAATAAGTA/ideoxyU/ (SEQ ID NO: 6) is complementary to the landing pad sequence for the next round and the well index.
The poly-A tail is used to stop priming and extension in the wrong direction. (See, for example, Kishi et al., and FIG. 2, step (b)).

In some embodiments, the first DNA hairpin is disabled at the end of each polymerase extension. In some embodiments, the hairpin can be disabled using, for example, an exonuclease or by treating the first DNA hairpin with enzymes that will remove a unique base present at the junction of the first oligonucleotide and the fourth oligonucleotide. In some embodiments, the exonuclease is, for example, but not limited to, lambda exonuclease or exonuclease VIII.

In some embodiments, the unique base is ideoxyU. In some embodiments, an ideoxyU is used to replace T at the 3′ end of the fourth oligonucleotide.

In some embodiments, the secondary nucleic acid tags are added by polymerase extension reaction, wherein the nucleic acid barcode bound to each target molecule-binding agent is further extended with one of the secondary nucleic acid tags by a strand displacing polymerase by providing a second DNA hairpin that comprises: (i) a fifth oligonucleotide, having sequence complementarity to the universal round 2 primer sequence, and (ii) a sixth oligonucleotide, wherein the sixth oligonucleotide further comprises a seventh oligonucleotide comprising a sequence encoding a secondary nucleic acid tag, and an eighth oligonucleotide comprising a sequence encoding a sequence complementary to the seventh oligonucleotide; wherein the seventh oligonucleotide is located at the 5′ end of the sixth oligonucleotide, and the eighth oligonucleotide is located at the 3′ end of the sixth oligonucleotide; and wherein the fifth oligonucleotide is fused to the 3′ end of the eighth oligonucleotide.

In some embodiments, each secondary nucleic acid tag comprises a unique well-specific second round barcode sequence at the 5′ end and a universal round 3 primer sequence at the 3′ end. In some embodiments, the sixth oligonucleotide further comprises a connecting sequence between the seventh and eighth oligonucleotides. In some embodiments, the connecting sequence is GGGCCTTTTGGCCC (SEQ ID NO: 1).

In some embodiments, the second DNA hairpin further comprises a polyA sequence at the 3′-end. In some embodiments, the polyA sequence is AAAAAAAA.

In some embodiments, the second DNA hairpin is disabled at the end of the second extension of the nucleic acid barcode. In some embodiments, the hairpin can be disabled using, for example, an exonuclease or by treating the second DNA hairpin with enzymes that will remove a unique base present at the junction of the fifth oligonucleotide and the eighth oligonucleotide. In some embodiments, the exonuclease is lambda exonuclease or exonuclease VIII. In some embodiments, the unique base is ideoxyU. In some embodiments, an ideoxyU is used to replace T at 3′ end of the eighth oligonucleotide.

In some embodiments, a nucleic acid tag described herein above comprises a unique barcode and a common 3′ region, e.g., a universal round 1 primer sequence, a universal round 2 primer sequence, a universal round 3 primer sequence or a universal subsequent round primer sequence. The nucleic acid tag is exemplified by the third oligonucleotide and the seventh oligonucleotide described herein above.

In some embodiments, different aliquots described herein above in each round are separately placed in different wells.

In some embodiments, the methods of the disclosure further comprise: step (g), which comprises repeating steps (d), (e), and (f), as set forth above, with the at least two secondary aliquots. In some embodiments, step (g) is repeated any number of times sufficient to generate a unique series of nucleic acid tags for each exosome, cell or vesicle in the sample. In some embodiments, step (g) is repeated any number of times sufficient to generate multiple unique nucleic acid tag for each exosome, cell or vesicle in a sample.

Accordingly in some embodiments, the methods of the disclosure further comprise repeating steps (d), (e) and (f) as set forth above, with any subsequent nucleic acid tags, such as third, fourth, fifth, sixth, seventh, eighth, ninth, tenth, and etc., to generate sufficient nucleic acid tags as desired.

In some embodiments, the number of times is about 20 to 100 times the number of the exosomes, cells or vesicles present in the sample.

DNA-Barcoded Antibodies (DBAs)

In some embodiments, the present methods use DNA barcoded antibodies (DBAs) against target proteins and UMIs, in combination with split-pool sequencing, to develop a novel single entity, e.g., vesicle, cell or exosome, phenotyping/proteomics platform, which can be applied to vesicle, cell or exosome profiling within patients. In some embodiments, the present method requires use of at least a target molecule-binding agent, e.g., antibody, with a conjugated DNA oligo. Each oligo contains an antibody specific barcode and a UMI. Each antibody binds specifically to its cellular target. During split-pool sequencing, all of the oligos associated with a given cell are identically labeled, yet no two cells apply the same tag to their oligos. During analysis, all oligos associated with a given cell are identified via their tag, and the composition and quantity of all bound antibodies is determined by analyzing the BC and UMI, respectively. See FIG. 5A-5D. For simplicity, only antibodies binding to cell surface proteins are shown in FIG. 5A-5D.

DBAs can comprise a library of >50 DBAs against extracellular and intracellular proteins. The target proteins comprise markers of immune cell identity or function (FIG. 5A). The specificities of the DBAs are determined by applying them to cell lines with previously defined markers (e.g., EBV immortalized B-cells, Sup-T1 cells) (FIG. 5B). The antibody staining, library preparation, DNA-sequencing and analysis pipelines such that antibodies that are directed against antigens present in a particular cell line show a 100-fold increase in abundance versus antibodies whose antigens are not expressed. Complex DBA libraries have been used for determination of protein contents within bulk cellular samples (e.g., fine needle aspirates from patients). The present method combines the validated DBAs with a split-pool sequencing method to specifically tag all the antibodies bound to a single cell with a common identifier. DNA-barcodes that have been specifically labeled with a unique cell tag are then PCR-amplified, sequenced, and analyzed, to determine the composition of antibodies bound to each cell (FIGS. 5C and 5D). As compared to other single cell, vesicle or exosome sequencing strategies, the use of a split-pool labeling approach removes the need for in-house specialized equipment while also decreasing the costs and greatly expanding the number of cells which can be analyzed within a single experiment. Split-pool sequencing is used for identifying short DNA oligos conjugated to antibodies.

A catalogue of well characterized antibodies against hundreds of human targets exists. These antibodies are able to bind to their corresponding antigen with exquisite sensitivity and specificity. By adding DNA-barcodes onto previously developed antibody reagents, the methods of the disclosure take advantage of the decades of effort and validation that have gone into creating high quality antibody reagents (Edfors, F., et al., Nature Communications. 2018; 8: 4130, the contents of which are hereby incorporated herein by reference). Adding DNA-barcodes to antibodies does not affect their target binding efficiency or specificity (Ullal, A. V., et al., Science Translational Medicine. 2014; 6(219): 219ra9, the contents of which are hereby incorporated herein by reference). By selectively conjugating defined DNA-barcodes to specific antibodies, an antibody binding to its antigen on a single cell can be detected by sequencing for the presence of the DNA-barcode attached to the antibody. In addition, UMIs have been shown to further improve the ability to precisely quantify the prevalence of a given element (e.g., a DNA-barcode) within a sample. This is possible because while all of the antibodies against a single antigen will share the same DNA-barcode, each antibody molecule will also have an additional sequence (the UMI) which is unique to an individual antibody molecule and thus enable a more precise quantitation of the number of antibodies bound to each cell. The combination of DNA-barcodes with UMIs drastically improves the information content of multiplex screens (Michlits, G., et al., Nature Methods. 2017; 14:1191-1197, the contents of which are hereby incorporated herein by reference).

In some embodiments, multiple samples comprising exosomes, cells or vesicles from multiple sources, can be processed in parallel. In such embodiments, each sample is incubated with distinct DNA-barcoded hashtag antibodies against universally expressed proteins (e.g., CD298 and (32 microglobulin) to uniquely tag each sample. A single or unique combinations of hashtag antibodies may be used to tag each sample. Methods of tagging are known and as described herein. The tagged samples are then pooled and used as the source of exosomes, cells or vesicles in the methods described above.

DBAs are a versatile tool for sensitive detection of proteins in vitro. They are made by conjugating short DNA oligos—either single- or double-stranded—to conventional antibodies used for immunohistochemistry. Traditional methods of conjugation used the biotin-streptavidin system but covalent conjugation can also be used. DBAs can be used in experiments for multiplexed protein analysis (M. Morishita, et al., Journal of Pharmaceutical Sciences, 2017; A. V. Ullal, et al., Science Translational Medicine, 2014; M. Stoeckius, et al., Nature Methods, 2017; and V. M. Peterson, et al., Nature Biotechnology, 2017, 35(10):936-939, the contents of which are hereby incorporated herein by reference), immuno-PCR (T. Sano, et al., Scientific Reports, 2016, the contents of which are hereby incorporated herein by reference), and FISH imaging (S. S. Agasti, et al., Chemical Science, 2017, the contents of which are hereby incorporated herein by reference). Due to the small size of exosomes, and consequently the low amounts of protein in a single exosome, sensitivity in detection is a critical consideration for the methods of the disclosure. DBAs fulfill this requirement because they allow antibodies to be counted by amplifying their signal using PCR and detected via next generation sequencing (NGS). DBAs also provide higher multiplexing capacity because they are not limited by overlapping emission/absorption spectra, which limit fluorescence based methods, allowing to probe dozens of proteins in parallel.

To produce DBAs, thiolated single-stranded oligos are used and conjugated to amines on lysine residues in the antibody via SM(PEG)₂ crosslinkers (S. S. Agasti, et al., Chemical Science, 2017, the contents of which are hereby incorporated herein by reference). Optimization will ensure an average of 1-2 oligos are conjugated to each antibody to minimize potential steric interference from the oligo on antigen binding and to ensure interpretable correlation between sequenced oligos and antibodies bound. To show that binding capacity of the antibody is unhindered by any oligo conjugation, flow cytometry results on cells incubated with oligo-coupled antibodies as primary antibodies are compared those on cells incubated with control primary antibodies. Specifically, the primary antibodies are first incubated with the cells expressing the target protein, further incubated with a secondary fluorophore-conjugated antibody against the primary antibodies, and quantified via flow cytometry.

Additionally, to ensure consistent stoichiometric conjugation between different antibodies, a control PEG molecule can be used to be detected on an SDS-PAGE gel to visualize the average number of conjugants (J. A. G. L. van Buggenum, et al., Scientific Reports, 2016, the contents of which are hereby incorporated herein by reference). The DBAs are incubated with protein A coated beads and bind to protein A coated beads with high affinity and specificity. Antibody-coated beads are then taken through the split-pool protocol. In some embodiments, the linkers between the oligo and the antibody are uncleavable. Alternatively, in other embodiments, the linkers between the oligo and the antibody are cleavable.

Split-Pool Sequencing

The methods of the disclosure can be scaled to any number of protein targets, and take advantage of several disparate technologies, i.e., DNA-barcoded antibodies, unique molecular identifiers, and split-pool sequencing.

Split-pool sequencing is a method of sequencing which involves serially ligating small tags onto DNA molecules which enables users to label all DNA molecules associated with a single entity, e.g., cell, vesicle or exosome, with a unique barcode (see, for example, Rosenberg, A., et al., Science. 2018; 360:176-182, the contents of which are hereby incorporated herein by reference).

Split-pool sequencing dramatically reduces the cost of single-cell sequencing (Rosenberg A B, et al., Science. 2018 Apr. 13; 360(6385): pp. 176-182, the contents of which are hereby incorporated herein by reference). The combination of DNA-barcodes with UMIs drastically improves the information content of multiplex screens. The methods of the disclosure describe a cost-effective, modular, and easy-to-use quantitative single entity protein profiling platform. For example, the present methods use DBAs and UMIs in combination with split-pool sequencing to improve information content and to reduce cost. The methods of the disclosure enable multiplexed and highly scalable detection of proteins at the single-entity level, but do not require in-house specialized equipment or specialized training.

The present disclosure also relates, at least in part, to a method of directly quantifying the abundance and state (e.g., phosphorylation state) of over a hundred proteins on tens of thousands of individual cells, exosomes, or vesicles, through the creation of a single-entity profiling platform which functions independently of specialized equipment. The methods of the disclosure enable comprehensive cellular phenotyping within a variety of diseases, and have innumerable translational applications in early stage discovery pipelines or in helping guide clinical decision making.

Other potential uses exist in the area of cellular therapies to better characterize the composition of modified cellular products or to delineate the properties of those cells that are most tumor invasive/effective.

Split-pool sequencing can be used to analyze over a hundred thousand cells, exosomes, or vesicles within a single experiment at a fraction of the cost of other single-entity sequencing methods which all require specialized equipment and proprietary reagents (e.g., Fluidigm C1, 1CellBio inDrop, 10× Genomics Chromium).

Computer program products can analyze data generated by any sequencing instrument. Non-limiting examples of sequencers include: a) DNA sequencers produced by Illumina™, for example, HiSeg™, HiScanSQ™, Genome Analyzer GAIIX™, and MiSeg™ models; b) DNA sequencers produced by Life Technologies™, for example, DNA sequencers under the AB Applied Biosystems™ and/or Ion Torrent™ brands; c) DNA sequencers manufactured by Beckman Coulter™; d) DNA sequencers manufactured by 454 Life Sciences™; and e) DNA sequencers manufactured by Pacific Biosciences™.

While some exemplary methods for sequencing are provided herein, these are exemplary and not meant to limit the scope of the present disclosure. Additional suitable methods for sequencing will be apparent to those of skill in the art based on the present disclosure in view of the knowledge in the art. Additional methods of sequencing are described in US Application No. 20150066385; Quail et al., 2012, BMC Genomics. 13 (1): 34; Liu et al., 2012, Journal of Biomedicine and Biotechnology. 2012: 1-11; each of which is incorporated herein by reference in its entirety.

Exosomes

In some embodiments, exosomes are analyzed using the methods of the disclosure. By “exosome” is meant any cell-derived, extracellular vesicle composed of a membrane enclosing an internal space, wherein the vesicle is generated from a cell by fusion of the late endosome with the plasma membrane or by direct plasma membrane budding, and wherein the vesicle has a longest dimension, such as a longest cross-sectional dimension, such as a cross-sectional diameter, ranging for example, from 10 nm to 150 nm, such as 20 nm to 150 nm, such as 20 nm to 130 nm, such as 20 nm to 120 nm, such as 20 to 100 nm, such as 40 to 130 nm, such as 30 to 150 nm, such as 40 to 150 nm, or from 30 nm to 200 nm, such as 30 to 100 nm, such as 30 nm to 150 nm, such as 40 nm to 120 nm, such as 40 to 150 nm, such as 40 to 200 nm, such as 50 to 150 nm, such as 50 to 200 nm, such as 50 to 100 nm, or from 10 to 400 nm, such as 10 to 250 nm, such as 50 to 250 nm, such as 100 to 250 nm, such as 200 to 250 nm, such as 10 to 300 nm, such as 50 to 400 nm, such as 100 to 400 nm, such as 200 to 400 nm, each range inclusive. As used herein, “inclusive” refers to a provided range including each of the listed numbers. Unless noted otherwise herein, all provided ranges are inclusive.

An exosome is typically created intracellularly when a segment of the cell membrane spontaneously invaginates and is ultimately exocytosed. As used herein, exosomes can also include any shed membrane bound particle that is derived from either the plasma membrane or an internal membrane. Exosomes can also include cell-derived structures bounded by a lipid bilayer membrane arising from both herniated evagination (blebbing) separation and sealing of portions of the plasma membrane or from the export of any intracellular membrane-bounded vesicular structure containing various membrane-associated proteins, including surface-bound molecules derived from the host circulation that bind selectively to the exosomal proteins together with molecules contained in the exosome lumen, including but not limited to mRNAs, microRNAs or intracellular proteins. Blebs and blebbing are further described in Charras et al, Nature Reviews Molecular and Cell Biology, Vol. 9, No. 11, p. 730-736 (2008). Exosomes can also include membrane fragments.

In some embodiments, size exclusion chromatography, such as gel permeation columns, centrifugation or density gradient centrifugation, and filtration methods can be used to isolate exosomes from a sample prior to use in the methods of the disclosure. Differential centrifugation, anion exchange and/or gel permeation chromatography, sucrose density gradients, organelle electrophoresis, magnetic activated cell sorting (MACS), or with a nanomembrane ultrafiltration concentrator can also be used to isolate exosomes. In a preferred embodiments, the purification method is a series of ultracentrifugation steps (Kowal, E. J. K., Ter-Ovanesyan, D., Regev, A. & Church, G. M. Extracellular Vesicle Isolation and Analysis by Western Blotting. Methods Mol. Biol. Clifton N.J. 2017, 1660: 143-152). Known methods for isolation of exosomes also include those disclosed in, for example, Monguio-Tortajada, et al., Cellular and Molecular Life Sciences (2019) 76:2369-2382, the contents of which are hereby incorporated by reference.

Highly abundant proteins, such as albumin and immunoglobulins, may hinder isolation of exosomes from a biological sample. Therefore, exosomes can be isolated by a system that utilizes multiple antibodies that are specific to the most abundant proteins found in blood. Such a system can remove up to several proteins at once, thus unveiling the lower abundance species such as cell-of-origin specific exosomes. Other known methods for exosome isolation include high abundant protein removal methods as described in Chromy et al. J. Proteome Res 2004; 3: 1120-1127. In another embodiment, the isolation of exosomes from a sample may also be enhanced by removing serum proteins using glycopeptide capture as described in Zhang et al, Mol Cell Proteomics 2005; 4: 144-155.

A detailed understanding of a patient's disease and response to treatment on a molecular level can help elucidate mechanisms of actions and inform downstream treatment follow-ups. The present disclosure includes methods of diagnosing disease, monitoring disease progression and monitoring response to treatment comprising non-invasive and unbiased tissue health surveillance by using already existing particles found in bodily fluids such as, for example, extracellular vesicles, including exosomes. To provide real-time, quantitative monitoring of cellular response through examining the patient's proteome, the present method is based on a new platform for single entity, e.g., cell, exosome or vesicle, proteomic analysis. The present disclosure relates, at least in part, to a method for split-pool sequencing coupled with DBAs in order to obtain a tissue specific surface proteome. The tissue specific surface proteome is an indicator of cellular homeostasis and can be used to monitor disease progression and treatment in a quantitative and sensitive fashion.

Exosomes are shed from all cells tested to date into bodily fluids which can be used to track changes in the proteome of all tissues within an organism at any given point in time. Specifically, exosomes are small extracellular vesicles secreted by many cell types (M. Li, et al., Philosophical transactions of the Royal Society of London. Series B, Biological sciences, 2014; and A. Vlassov, et al., Biochim. Biophys. Acta, 2012, the contents of which are hereby incorporated herein by reference) and found in all bodily fluids including urine (M. Li, et al., Philosophical transactions of the Royal Society of London. Series B, Biological sciences, 2014, the contents of which are hereby incorporated herein by reference), blood (M. Li, et al., Philosophical transactions of the Royal Society of London. Series B, Biological sciences, 2014, the contents of which are hereby incorporated herein by reference), cerebrospinal fluid (D. Chiasserini, et al., Journal of Proteomics, 2014, the contents of which are hereby incorporated herein by reference), and amniotic fluid (A. Asea, et al., Journal of Reproductive Immunology, 2008, the contents of which are hereby incorporated herein by reference). They are characterized by several highly expressed surface transmembrane markers including CD9, CD81, and CD63 (M. Simons, et al., Current Opinion in Cell Biology, 2009, the contents of which are hereby incorporated herein by reference). Exosome biogenesis follows the endosomal pathway. Endosomes are intracellular compartments that have an inverted membrane composition from that of the cellular plasma membrane. Molecules from the plasma membrane are sorted in the endosome for recycling or degradation in the lysosome mediated by an ESCRT-dependent pathway. In order for the release of exosomes to occur, the endosomal compartments must bud into the lumen of the endosome turning it into a multivesicular body (MVB). If the MVB fuses with the lysosome, the contents are degraded. However, if the MVB fuses with the plasma membrane, the vesicles are then released to the extracellular space. The secreted vesicles are known as exosomes. Due to their means of biogenesis it is not surprising that several studies have shown that exosome surfaces are rich in membrane proteins reflective of the cell.

Exosomes have been shown to contain cellular components such lipids, proteins, RNA, and DNA. Exosomes are important for cell signaling; intact mRNA transported from one cell via exosome to another has been shown to be able to be used in protein translation (H. Valadi, et al., Nature Cell Biology, 2007, the contents of which are hereby incorporated herein by reference). Network based gene analysis has shown that these mRNA transcripts are enriched for such functions as cellular development, protein synthesis, RNA post-transcriptional modification. Exosomes also may play an important role in immune response. Exosomes secreted by tumor cells can be captured by dendritic cells and used to present antigens to activate the immune system (0. G. de Jong, et al., Journal of Extracellular Vesicles, 2012; Q. Lian, et al., Cell Research, 2017; and F. M. Barros, et al., Frontiers in Immunology, 2018, the contents of each of which are hereby incorporated herein by reference). Concurrently, other small molecules, like PD-L1 which inhibit T-cell response, have been shown to be encapsulated in exosomes which dampen immune response (F. M. Barros, et al., Frontiers in Immunology, 2018; and Y. Yang, et al., Cell Research, 2018, the contents of each of which are hereby incorporated herein by reference). In addition, studies have also shown that exosomes serve as a method of secretion for harmful cytoplasmic DNAs (A. Takahashi, et al., Nature Communications, 2016, the contents of which are hereby incorporated herein by reference).

Because of the ubiquitous property that nearly all cells shed exosomes into extracellular spaces, they present a versatile tool for interrogating a diverse panel of tissues simultaneously. Taking advantage of the fact that the plasma membrane proteins of exosomes are representative of the membrane proteins of the parent cell, monitoring proteome alterations of exosomes are a convenient, non-invasive way to monitor cell surface proteome alterations of tissues. Exosomes also have the ability to cross the blood-brain barrier, carrying with them information about brain tissue health, a property now being explored for diagnosing brain diseases from exosome analysis (K. M. Kanninen, et al., Biochimica et Biophysica Acta—Molecular Basis of Disease, 2016, the contents of which are hereby incorporated herein by reference).

Exosome diagnostics in the clinical setting have largely relied on identifying specific markers for disease from a bulk population of purified particles. The most obvious limitation with this strategy is its lack of sensitivity. A more recent technology has demonstrated that exosomes and other secreted vesicles have the potential to be powerful tools in monitoring cancer therapy response (H. Shao, et al., Nature Medicine, 2012, the contents of which are hereby incorporated herein by reference). Expanding upon this result, exosomes are probed for their tissue specific proteome in order to diagnose and monitor the progression of disease. In some embodiments, the present disclosure provides methods for determining the proteomic profile of individual exosomes are obtained by combining single entity sequencing technologies and DBAs.

The natural exosome biogenesis incorporates cellular material, especially membrane proteins from the parent cell, and is a reflection of the biological status of the cell of origin (H. Shao, et al., Nature Medicine, 2012; K. L. Schey, et al., Methods, 2015; H. Im, et al., Nature Biotechnology, 2013; 0. G. de Jong, et al., Journal of Extracellular Vesicles, 2012; and B. Gyorgy, et al., Cellular and Molecular Life Sciences, 2011. F., the contents of each of which are hereby incorporated herein by reference). In some embodiments, the methods of the invention provide for sequencing the antibodies bound to single exosomes to detect changes in the exosome surface proteome in order to quantitatively monitor changes in cell state from multiple organs in parallel. However, because exosomes are 100 times smaller in diameter than the average mammalian cell, it is anticipated that making definitive conclusions about cell state from the sparse data obtained from a single exosomes may be challenging in some instances. Thus, in some embodiments, pooling exosomes with the same tissue-specific markers, allows for generation of a collective surface proteome for the tissue of interest. The methods of the present disclosure are a more time-sensitive diagnostic tool that are superior to current serum-based diagnostic standards. Furthermore, the methods of the disclosure can be used for studying any pathology of interest.

Diagnostic and Therapeutic Methods

In some aspects, the methods and systems of the disclosure are applied to discovery pipelines to identify pathways of interest (e.g., in a preclinical setting) and in a clinical setting for diagnostic, monitoring, and/or therapeutic applications for a variety of diseases and disorders. Thus, in some embodiments, the present methods of the disclosure are used to analyze samples obtained from patients with disease (e.g., cancer, organ transplant, and autoimmune disease). In some embodiments, the samples are compared to control samples, e.g., those obtained from healthy subjects or other control samples, to diagnose disease or monitor response to therapy.

In some aspects, the present disclosure is directed to methods for diagnosing a disease or disorder, e.g., cancer, autoimmune disease or inflammatory disease, determining a patient's response to a therapy, and/or monitoring the patient for undesired side effects to the therapy, the method comprising: (a) contacting exosomes, cells or vesicles with a plurality of target molecule-binding agents, wherein each target molecule-binding agent comprises a nucleic acid barcode and optionally a unique molecular identifier (UMI), wherein target molecule-binding agents that are specific to an identical target molecule share an identical nucleic acid barcode, wherein the exosomes, cells or vesicles are isolated from blood of a patient suffering from a disease or disorder, e.g., cancer, autoimmune disease or inflammatory disease, before or after the patient has been treated with a therapy, wherein the plurality of target molecules comprise molecules that are markers indicative of a disease or disorder, e.g., cancer, autoimmune disease or inflammatory disease, efficacy of the therapy, and/or are indicative of undesirable side effects, and wherein expression levels of the target molecules determined in the patient before or after the therapy are compared with expression levels of the corresponding target molecules determined in normal controls, thereby diagnosing a disease or disorder, e.g., cancer, autoimmune disease or inflammatory disease, determining the patient's response to a therapy, and/or monitoring for undesired side effects of a therapy.

In some embodiments, the patient's response to therapy is determined and/or monitored in real-time.

In some embodiments, the patient is further treated with the same therapy, if the therapy is effective, but associated with little or no undesired side effects; or the patient is treated with a different therapy if the therapy is not effective and/or is associated with undesired side effects.

Real-Time Cancer Diagnostics

Chemotherapy is a pillar of the clinical oncology arsenal. Each year over half a million Americans are treated with chemotherapy, a collection of toxic small molecules and proteins designed to selectively destroy tumor cells (DeVita V. T. Chu, EA history of cancer chemotherapy. which are hereby incorporated herein by reference). For all patients, the choice of therapy is largely based on historical experience and tumor molecular profile (Santos, F. N., de Castria, T. B., Cruz, M. R. S. & Riera, R. Cochrane Database Syst. Rev. CD010463, 2015; and Ahmadzada, T. et al. J. Clin. Med. 2018, 7, the contents of each of which are hereby incorporated herein by reference). Given the sizable genetic, expression and cellular heterogeneity between patients with the same tumor classification, it is not surprising that patients treated with the identical chemotherapeutic regimen can have vastly different responses. Despite recent advances in precision medicine, patients still regularly fail to respond to treatment, and nearly all patients undergoing chemotherapy suffer from the undesirable “side-effects” ranging from extreme nausea and diarrhea to more severe complications such as heart failure or life-threatening bacterial infections (Pearce, A. et al., PloS One 12, e0184360; Thatcher, N. First- and second-line treatment of advanced metastatic non-small-cell lung cancer: a global view. BMC Proc. 2008, 2 Suppl 2: S3; Cardoso, F. et al. Ann. Oncol. Off. J. Eur. Soc. Med. Oncol. 2002, 13: 197-207; and Catalano, V. et al. Br. J. Cancer 2008, 99: 1402-1407). In fact, it is not uncommon for a course of chemotherapy to show little to no effect on tumor growth, yet during the treatment, the patient is still forced to suffer from undesired side-effects (Temel, J. S. et al. N. Engl. J. Med. 2010, 363: 733-742; and Harrington, S. E. & Smith, T. J. JAMA 2008, 299: 2667-2678, the contents of each of which are hereby incorporated herein by reference). Given the sizable cost of therapy and the life-threatening complications that can ensue, it is of critical importance to maximize the benefits that patients receive from chemotherapy (Pritzker, K. P. H. Expert Rev. Mol. Diagn. 2015, 15: 971-974, the contents of which are hereby incorporated herein by reference).

The present disclosure describes a diagnostic system that reports a patient's response to therapy in real-time, while also monitoring for undesired side-effects. The present disclosure describes a method of SESeq that can be applied to real-time monitoring of patients' response to therapy.

Exosomes, as small extracellular vesicles containing RNA, are secreted into the circulation by all cells, including tumor cells (Momen-Heravi, F., Getting, S. J. & Moschos, S. A. Pharmacol. Ther. 2018, the contents of which are hereby incorporated herein by reference). Blood contains a mixture of extracellular vesicles from various tissues, so in order to gain insight into tumor and normal tissue response to chemotherapy, extracellular vesicles need to be sequenced individually. In some embodiments, single entity sequencing is used to selectively analyze the contents of individual extracellular vesicles isolated from blood in a cancer patient. The present disclosure describes a non-invasive technology to allow clinicians to dynamically tailor patient therapy to tumor response while also enabling them to catch undesired off-target effects before they become clinically evident.

Since extracellular vesicles are secreted by all cells into the circulation and contain RNA from their cell of origin, it is hypothesized that individually sequencing the contents of each extracellular vesicle will provide insights into the cellular processes occurring within both the tumor and normal tissues. The present disclosure includes methods and analysis pipelines for performing single entity sequencing, e.g., SESeq. In some embodiments, single entity sequencing, e.g., SESeq is used to determine the response of tumor and normal cells to chemotherapy within simulated patient samples. The present method is the a real-time method for individually analyzing the contents of extracellular vesicles and used this data to infer the cellular state of tumor and normal cells, thereby transforming cancer care by monitoring efficacy of chemotherapy against tumor while helping better detect and manage undesired side-effects.

The present disclosure is directed, at least in part, to a scalable method with high levels of sensitivity and scalability and requires combining split-pool sequencing and DBA technologies for quantifying the abundance of dozens of proteins on individual extracellular vesicles. Specifically, by coating single extracellular vesicles with a library of DBAs followed by sequencing all antibodies bound to each extracellular vesicle, a set of specific proteins in each extracellular vesicle can be identified. This information can be amalgamated to understand which tissue the extracellular vesicle came from and the state of that tissue at the time the extracellular vesicle was released. The platform's versatility lends itself to be a good tool for not only diagnostic purposes but also for downstream treatment monitoring or even off-target drug effect discovery. A single extracellular vesicle proteome sequencing platform allows a physician to gain dynamic biological insight into disease progression in a quantitative manner that surpasses current standards of disease monitoring.

In order to attain single extracellular vesicle resolution in sequencing, a split-pool barcoding is used as a sequencing approach. Split-pool barcoding relies on randomly distributing a mixture of cells each containing a series of in situ transcribed cDNA into one of 96 wells. Each well contains a unique DNA primer that is ligated onto all of the cDNA molecules associated with each cell. After the ligation, all wells are pooled and the process is repeated several more times ultimately tagging all DNA molecules in a given cell with a unique barcode combination.

All publications, patents and patent applications cited in this specification are herein incorporated by reference in their entirety for all purposes as if each individual publication, patent, or patent application were specifically and individually indicated to be incorporated by reference for all purposes. The publications discussed herein are provided solely for their disclosure prior to the filing date of the present application. Nothing herein is to be construed as an admission that the inventors described herein are not entitled to antedate such disclosure by virtue of prior disclosure or for any other reason.

The following Examples are set forth as being representative of the present disclosure. These examples are not to be construed as limiting the scope of the disclosure as these and other equivalent embodiments will be apparent in view of the present disclosure, figures, tables and accompanying claims.

### Examples

### Example 1: Sequencing the Surface Proteome of Single Cells Using DBAs

This example describes methods of sequencing proteins on cells. However, it is applicable to exosomes and vesicles as well.

Two cell types (human liver-HEPG2 and human glioblastoma (GBM)-LNZ308) and a panel of 6-8 antibodies specific to each of the two cell types are chosen. The panel of the antibodies are used to demonstrate that a single cell protein quantification method using split-pool sequencing. Liver tissue can be chosen through an analysis of the Human Protein Atlas database (M. Uhlen, et al., Science, 2015). Liver tissue is selected because it exhibits the highest number of tissue specific membrane proteins and because it plays roles in drug metabolism and toxin filtration. A human glioblastoma cell line is chosen because bulk glioblastoma exosomes have previously been shown to allow real time monitoring of GBM therapy (H. Shao, et al., Nature Medicine, 2012).

Antibodies to these proteins will be procured and validated for specificity by CRISPR knockout or knockdown of their targeted antigen and finally conjugated to oligos for single cell antibody protein profiling sequencing. Two cell types in a mixed tissue experiment are co-cultured and incubated with DBAs for labeling prior to taking them through the split-pool pipeline. In the two-tissue experiment, clean single cells are obtained through split-pool without particle aggregation. It will be confirmed whether the cell type of each of the single cells is profiled with high accuracy. Specifically, it will be confirmed whether the single cell protein profiles show correlation of protein expression levels between cells of the same lines, providing an indication of the variance to be expected in these experiments.

As long-term passaging of immortalized cell lines tend to be quite homogeneous as compared to the native tissue. Within-cell comparisons will allow testing of the dynamic range of the methods of the disclosure. In order to test that the results derived from the aforementioned split-pool sequencing method are accurate and representative of single cell populations, the integrity and accuracy of the results is validated with a lower throughput single cell sequencing method based on the Chromium 10×.

### Example 2: Sequencing the Proteome of Single Exosomes Using DBAs

Experiments on exosomes present additional challenges to whole cell experiments. Because exosomes are much smaller, the protein information is much more sparse, it is expected that the number individual exosomes to be processed will be much larger than in traditional single cells experiments-on the scale of tens to hundreds of thousands. Two exosome populations are mixed and are incubated with DBAs for labeling prior to being taken through the split-pool pipeline. It will be confirmed that the tissue origin of each of the exosome is profiled with high accuracy. Specifically, it well be confirmed if the surface proteome profile of individual exosomes correlates well with the surface proteomes of individual cells generated from whole cell profiling.

Studies have shown high degrees of correlation between cell surface proteome and bulk exosome surface proteome in GBM, colorectal, and B-cell leukemia cell lines (H. Shao, et al., Nature Medicine, 2012; and L. Belov, et al., Journal of Extracellular Vesicles, 2016). It is predicted that the methods of the disclosure will enable pooling of single exosomes identified from each cell type and reconstruction of two tissue specific proteomes that have good surface proteome correlations with their parent cell line. From single exosome data generated from split-pool, the antigen density on the surface of exosomes will be determined and the sensitivity detecting protein level changes for downstream applications will be determined. If it is confirmed that the specificity of tissue origin of exosomes can be readily called, a layer of complexity will be added by titrating a third exosomal population, which is obtained from neither glioblastoma nor liver.

The method developed in this Example will be used to establish a library of DBAs for single exosomes isolated from different tissues of origin and optimize an experimental pipeline for sequencing DBAs from single exosomes and correctly calling their tissues of origin.

### Example 3: Acetaminophen Toxicity has a Detectable Phenotype in Cell Culture and can be Similarly Observed at the Bulk Exosome Level

HepG2 cells are cultured and treated with 1004 to 10 mM of acetaminophen for 24-48 hours (I. Manov, et al., Basic & Clinical Pharmacology & Toxicology, 2004; and R. H. Pierce, et al., Biochemical Pharmacology, 2002). A bulk RNA sequencing analysis is done on both the drug-treated and untreated cell lines as well as exosomes harvested from the media of the drug-treated and untreated cells. In addition, mass spectrometry is performed on drug-treated and untreated HepG2 cells as well as their respective exosome populations to confirm similarity in their protein expression levels under drug stress. It will be confirmed whether these protein changes match already known results in literature a comprehensive list of hits that may be specific to exosomes will be generated.

It is predicted that some the differentially expressed genes after drug treatment will match hits found in the literature because HepG2 cells are one of the most widely used cell lines to study acetaminophen toxicity (J.-M. Prot, et al., “Toxicology and Applied Pharmacology, 2012; and R. M. Rodrigues, et al., “Data in Brief 2016). Though many of the existing datasets that study acetominophen toxicity are from microarray experiments, it is possible to confirm these results with bulk RNA sequencing experiments. These results will also be confirmed by mass spectrometry. It is expected that the hits from mass spectrometry and those identified in RNA sequencing will show correlated modulation in response to drug toxicity. If, in fact, it is found that proteomic profiling results do not correlate with RNA sequencing, protein data generated from the mass spectroscopy will be used as markers of acetaminophen toxicity. From these pure, bulk samples, it is possible ascertain the signal to expect from exosome samples obtained from downstream animal experiments.

### Example 4: Performing Single Exosome Sequencing on a Complex Fluid to Confirm the Sensitivity Limits

It will be shown that single exosomes of liver or brain origin can be detected. Blood is extracted from mice. Exosomes from blood will be isolated using the ultracentrifugation method and subsequently incubated with prepared DBAs. Samples will be taken through the split-pool sequencing pipeline and analysis.

It is predicted that the single exosome population detected in the mixed complex bodily fluid will have generally similar expression patterns to that of the pure population isolated from cell culture supernatant. However, it is expected that immortalized cell lines do not exactly recapitulate the state of cells in vivo, therefore a first round of complex fluid analysis is performed with a spiked in population of cell culture supernatant exosomes that have been previously analyzed to confirm robustness of the assay. Because many of the antibodies to be used in identifying the tissue origin of the exosome will be vetted for specificity, there should be minimal binding to off-target tissues. Nevertheless, it is inevitable that there will be noise from non-specific antibody binding, ligation, or PCR amplification. It is predicted that signals coming from biological and technical artifacts will be relatively low and have a discernible profile compared to the true signal.

Based on this experiment, it is possible to find a pattern for the noise generated in the sequencing of complex fluids and apply some filtering rules to discriminate between signal and noise for downstream experiments. The complexity of the exosome sample can be reduced by enriching for particular exosomal fractions via antibody-based pre-enrichment before applying DBAs to the samples.

### Example 5: Single Exosome Antibody Sequencing is a Sensitive Readout of Pathology in an Animal Model of Disease

An animal model of acetaminophen toxicity is used to study the time dependent change of liver exosome proteomic profiles. For this study, a cohort of animals are administered acetaminophen and terminal bleeds are performed at 0, 6 and 24 hours post treatment to extract exosomes. In parallel, AST and ALT levels are assayed in the blood, along with examination of liver histology and performance of RNA-seq on liver tissue. Histology and RNA-seq provide independent methods for tracking liver function and health over the course of the experiments. These data will allow assessment of how well methods of the disclosure compares to traditional tests, while also enabling testing of whether the present method shows additional performance characteristics not seen with blood based tests of AST/ALT.

It is predicted that the exosome proteomic profiles will show improved sensitivity to up-regulation of protein markers that respond to liver toxicity. It is also predicted that sensitivity in post-treatment recovery will be improved. Whereas enzyme levels may be slow to decrease in the circulating bloodstream-ALT/AST have half-lives ranging from 14-72 hours (E. G. Giannini, et al., CMAJ, 2005)—exosomes biogenesis may occur on a faster timescale (M. Morishita, et al., Journal of Pharmaceutical Sciences, 2017)—exosomes have been shown to be absorbed with a half-life of 2-4 minutes- and allow to track organ function in near real time as compared to traditional enzyme tests.

### Example 6: Performing Single Exosome Sequencing (SESeq)

Exosomes are harvested from two well-characterized and easily-manipulatable cell lines, human embryonic kidney 293T cells and mouse 3T3 fibroblasts (Todaro, G. J. & Green, H. J. Cell Biol. 1963, 17: 299-313; and Graham, F. L, Smiley, J., Russell, W. C. & Nairn, R. J. Gen. Virol. 1977, 36: 59-74). Pure populations of exosomes are isolated by a series of ultracentrifugation steps and are characterized for purity using both nanoparticle tracking analysis and scanning electron microscopy (Kowal, E. J. K., Ter-Ovanesyan, D., Regev, A. & Church, G. M. Methods Mol. Biol. Clifton N.J. 2017, 1660: 143-152; and Rupert, D. L. M., Claudio, V., Lasser, C. & Bally, M. Biochim Biophys. Acta, 2017, 1861:3164-3179). Upon obtaining pure exosome populations from human embryonic kidney 293T cells and mouse 3T3 fibroblasts, equal quantities of two populations of exosomes are mixed together. To detect the proteins present on each exosome, the mixture of exosomes are incubated with a panel of DBAs, which can be used to quantify the level of hundreds of proteins of interest using conventional single cell sequencing pipelines (M Stoeckius, et al., Nat. Methods 2017, 14: 865-868). This exosome mixture, taken as an input into the Columbia Genome Center's Chromium controller (10× Genomics), and each exosome is individually encapsulated and reverse transcribed. All RNAs from the same exosome are tagged with a common DNA barcode (FIG. 6A-6B). The Chromium-generated libraries will then be run on the Illumina NextSeq 500 or NovaSeq sequencing platform. Because human and mouse genomes have large differences in character (neuronal-like versus fibroblast and organism origin, i.e., human versus mouse), it can be readily determined if the method of performing SESeq is successful, as the system should report only human or only mouse-specific RNA-sequences and antibody barcodes associated with each individual exosome.

Once SESeq protocol and analysis pipeline using mixtures of mouse and human exosomes are established, it will be applied to distinguishing between exosomes secreted from different types of human tissue. Specifically, exosomes from 8 immortalized human cell lines derived from various tissues (e.g., HUVEC for vascular endothelium, Ker-CT for skin, RPTEC for kidney tubular epithelium) will be isolated and then SESeq performed on the exosomes from each cell line individually and on exosome pools containing exosomes from 2, 4 or 8 lines. Sequencing data from the exosome pools will then be clustered in the absence of any knowledge of the number of input samples (de novo clustering) or clustered using the reference SESeq data obtained when the exosomes from each cell line were sequenced individually (reference-based clustering). Finally, each cell line chosen will also be sequenced using conventional bulk RNA sequencing to better understand how well SESeq recapitulates the transcript diversity within each cell line.

Methods for exosome isolation and manipulation, next-generation sequencing, and clinical laboratory diagnostics required for successfully developing the SESeq platform are described in, for example, Alvarez, M. J. et al. Nat. Genet. 2016, 48: 838-847; Ding, H. et al. Nat. Common. 2018, 9: 1471; Guo, X. et al. Nat. Biotechnol. 2018, 36: 540-546; Bester, A. C. et al. Cell 2018, 173: 649-664.e20; DiCarlo, J. E., et al., Safeguarding CRISPR-Cas9 gene drives in yeast. Nat. Biotechnol. 2015, 33: 1250-1255; Chavez, A. et al., Nat. Methods 2015, 12: 326-328; and Yeo, N. C. et al., Nat. Methods 2018, 15: 611-616.

If upon running the SESeq pipeline, it is found that there are too few transcripts per exosome to be able to identify the cell of origin for the majority of exosomes, this can be overcome by 1) adapting single cell clustering algorithms, such as the metaVIPER, that are specifically designed to deal with issues of data sparsity such as gene dropout which violate many of the assumptions made by orthodox statistical tools; 2) modifying the exosome isolation procedure to capture various sub-fractions by size or density to determine if a particular fraction is most suitable for SESeq analysis (exosomes are a fraction of a heterogeneous group of extracellular vesicles) (Collino, F. et al. Stem Cell Rev. 2017, 13: 226-243; 3) adapting other methods such as SPLiT-seq which are able to capture >10× more events than methods using commercial devices at a similar costs (Li, M. et al. Philos. Trans. R. Soc. Lond., B, Biol. Sci. 2014, 369; Alvarez, M. J. et al., Nat. Genet. 2016, 48: 838-847; Ding, H. et al. Nat Commun 2018, 9: 1471; Collino, F. et al. Stem Cell Rev 2017, 13: 226-243; and Rosenberg, A. B. et al. Science 2018, 360: 176-182).

### Example 7: Determining the Response of Tumor and Normal Cells to Chemotherapy within Simulated Patient Samples

To establish methods of using SESeq to infer cellular state from both tumor and normal cells, human A549 non-small cell lung cancer (NSCLC) cells and RPTEC kidney cells are treated with paclitaxel or cisplatin. Exosomes will be collected from untreated and treated cells 4, 12, 24 and 48 hours after addition of drug, and SESeq will be performed on each sample. In addition, to better understand the relationship between contents of RNA and protein in exosomes and cellular state, conventional RNA sequencing and unbiased proteomics will be also performed on the A549 and RPTEC cells at all collection times.

Paclitaxel and cisplatin have been chosen for these studies because they are commonly used in first line lung cancer treatment regimens, show varied efficacy within patients, have known nephrotoxic side-effects, and have diverse mechanisms of action (Malhotra, V. & Perry, M. C. Cancer Biol. Ther. 2003, 2: 1-3; Lameire, N. Clin. Kidney J. 2014, 7: 11-22; Perazella, M. A. Clin. J. Am. Soc. Nephroi. CJASN7, 2012, 1713-1721; and Ettinger, D. S. et al. Non-Small Cell Lung Cancer, Version 5.2017, NCCN Clinical Practice Guidelines in Oncology. J. Natl. Compr. Cancer Netw. JNCCN 2017, 15: 504-535).

Using the data obtained from SESeq, the cellular state of the A549 or RPTEC cells at each of the time points (e.g., cell cycle arrested or activating DNA damage response) will be inferred, and correlation with bulk cell analysis will be determined. Finally, to test the utility of SESeq in a more clinically-relevant setting, exosomes from the blood of several healthy human donors are isolated and spiked in varying amounts of A549 or RPTEC derived exosomes from paclitaxel-treated, cisplatin-treated or non-treated cells. Each sample will undergo SESeq, and a researcher blind to the composition of each sample will determine 1) if A549 and/or RPTEC cell-derived exosomes were added to the sample and 2) if the A549 and/or RPTEC cells were treated with drug.

It is predicted that it is possible to assign individual exosomes to a particular tissue by sequencing all RNAs along with determining the abundance of upwards of a hundred different proteins through the use of DBAs within the exosomes (Li, Y., Zhang, Y., Qiu, F. & Qiu, Z. Electrophoresis 2011, 32: 1976-1983; and Lin, J. et al. Scientific World Journal 2015, 2015: 657086). Once exosomes have been assigned, the data is pooled from all exosomes that have been assigned to a given tissue. Markers of cell state, such as transcription of stress response gene, or increased presence of markers of apoptosis, such as phosphatidylserine on the surface of the exosomes, are identified.

### Example 8: Determining the Response of Tumor in a Mouse Model to Chemotherapy

Mouse models of NSCLC (Kwon, M. & Berns, A. Mol. Oncol. 2013, 7: 165-177) are prepared by injecting human A549 cells to mice. Once tumors have formed within the animals, the animals will be treated with a variety of drugs, some of which have previously been shown to cause tumor regression and others that are known to not affect tumor growth. At the start of the experiment and at various time points after initiating treatment, exosomes will be isolated from the blood of animals to determine the efficacy of drug treatment on the tumor. In addition, as all tissues within the animal are experiencing the effects of chemotherapy, insights gained from SESeq are analyzed to determine correlation with potential complications of treatment, such as cardiotoxicity, nephrotoxicity or gut epithelial dysfunction.

### Example 9: Determining the Response of Tumor in a NSCLC Patient to Chemotherapy

A study will be performed using SESeq on samples from NSCLC patients being treated with chemotherapy to determine whether insights gained from SESeq correlate with clinical outcome (e.g., patients predicted by SESeq to have tumors responding to treatment show decrease in tumor mass or patients predicted to be suffering from cardiomyocyte stress show increase in circulating cardiac troponin-1). To date, there are no universal solutions for rapidly determining a patient's response to treatment. Commonly used methods such as secreted tumor antigen are not available for the majority of tumors and also suffers from issues of specificity (Adhyam, M. & Gupta, A. K. A Review on the Clinical Utility of PSA in Cancer Prostate. Indian J. Surg. Oncol. 2012, 3: 120-129). On the opposite end of the spectrum, serial imaging can be used to track nearly any tumor over time, yet it suffers from low assay sensitivity and at times requires weeks to months between imaging to confidently determine if there has been a change in a patient's tumor burden (Weber, W. A. J. Nucl. Med. Off. Publ. Soc. Nucl. Med. 2009, 50 Suppl 1: 1S-10S).

SESeq can be used as a diagnostic for following a patient's response to therapy (see FIG. 8). Finally, by obtaining transcriptional information on not just tumor cells but all cells that secrete exosomes, SESeq can enable clinicians to better determine patients at high risk of deleterious drug side-effects, allowing patients to switch to an alternative chemotherapeutic regimen before suffering from irreversible toxicity.

### Example 10: Multiplex Quantification of Protein Expression on Single Cells

Measurement of cellular protein levels has been important in various aspects of biology discovery. The most popular method for detecting proteins on single cells, flow cytometry, is limited by considerations of fluorescent spectral overlap. While mass cytometry (CyTOF) allows for the detection of up to 50 epitopes simultaneously, it requires local access to specialized instrumentation not commonly accessible to many laboratories. One embodiment of the present invention, PSP-seq, overcomes these limitations. PSP-seq enables multiple protein targets on single cells to be quantified without the need for specialty equipment other than access to widely available next generation sequencing (NGS) services. Studies demonstrate that PSP-seq compares favorably to traditional flow-cytometry, and allows for measurement of over two dozen target proteins to be assayed at a single cell level. To showcase the potential of PSP-seq, peripheral blood and bone marrow aspirates from human clinical samples was analyzed, and identified pathogenic cellular subsets with high fidelity. The ease of use of this new technique makes it a promising technology for high-throughput proteomics and for interrogating complex samples, such as those from patients with leukemia.

Materials and Methods

Cell Culture and General Staining Procedure

Jurkat cells were cultured in RPMI media (Gibco) with 10% FBS (Gibco) and 1% Pen Strep (100× concentrated stock, Invitrogen). HEK293T cells (obtained from ATCC) were cultured in DMEM (Gibco) with 10% PBS and 1% Pen Strep. Cells were maintained in T225 flasks at 37° C. in a humidified atmosphere with 5% CO2. HEK293T cells were harvested by gentle trypsinization with 0.05% Trypsin-EDTA (Gibco) for 2 minutes at room temperature. Cells were counted on a hemocytometer and combined at a 1:1 ratio. A total of 1 million cells were then washed with PBS before blocking with 1% BSA and Fc-block at manufacturer's recommended dilution (BD Biosciences) for 30 minutes at 4° C. on a rotator.

Staining of Jurkat and HEK293T Cells

All DNA-barcoded antibodies were obtained from Biolegend and are in TotalSeqB format. An antibody cocktail of CD56, CD155, CD29, CD4, CD45, CD28, and isotype control was created by mixing the antibodies at equal ratios. Cells were incubated with a total of 1.5 μg of the antibody mixture per 1 million total cells in a staining volume of 100 μl of blocking solution at 4° C. for 1 hour. Stained cells were washed with 1 ml of ice-cold PBS and then centrifuged for 5 minutes at 300 g. Four more washes were then performed under the same conditions. Finally, stained cells were fixed using 4% PFA for 10 minutes at room temperature and washed once with ice-cold PBS before depositing them into wells for their first round of well barcode ligation.

Jurkat CD4 Expression Cell Sorting

Jurkat cells were harvested, counted on a hemocytometer, and resuspended at a density of 1 million cells in 100 μl of blocking buffer. Cells were blocked for 30 minutes at 4° C. on a rotator. A total of 10 million cells were stained with an antibody cocktail containing several DNA-barcoded antibodies along with anti-CD4 antibodies that were conjugated to either a DNA-barcoded or FITC. The antibody cocktail was composed of 1 μg of each DNA-barcoded anti-CD4, anti-CD155, and anti-CD29 as well as 2 μg of anti-CD4-FITC. Cells were incubated with antibodies for 1 hour at 4° C. on a rotator and washed 5 times with cold PBS followed by centrifugation. Cells were then sorted based on FITC fluorescence levels into three bins, high, medium and low. After sorting, each population of cells were stained with a hashtag antibody to label their CD4-FITC expression level. Individual cell populations were counted and resuspended to a density of 1 million cells in 100 μl of blocking buffer before staining with 0.5 μg of the respective hashtag antibodies. Re-stained cells were washed 5 more times to remove unbound antibodies before fixing in 4% PFA in PBS and taken through the ligation barcoding steps.

Staining of Patient Samples

Whole blood or bone marrow aspirate from deidentified clinical samples were obtained. Approximately 2 ml of either whole blood or bone marrow aspirate from each sample was first stained with 0.5 μg of hashtag antibody for 30 minutes at 4° C. on a rotator (Table 1). Peripheral blood mononuclear cells (PBMCs) were then isolated with Ficoll-Paque according to the manufacturer's protocol. Cells were counted using a hemocytometer and combined stoichiometrically by cell number—of note several samples had low cellularity and were not able to be equally sampled. A total of 1 million cells were then blocked with 1% BSA and Fc-block (according to manufacturer's protocol). Cells were then stained with a total of 4 μg of a panel of antibodies (Table 2) in 100 μl volume of blocking buffer before being washed five times by centrifugation at 300 g and 4° C. Stained cells were fixed using 4% PFA for 10 minutes and washed once before ligation barcoding.

TABLE 1
Hashtag Legend

| | Hashtag | | | |
|---|---|---|---|---|
| Sample | antibody | Catalog # | | SEQ ID |
| ID | identifier | (Biolegend) | Barcode sequence | NO: |
| 1 | HT1 | 394631 | GTCAACTCTTTAGCG | 7 |
| 2 | HT2 | 394633 | TGATGGCCTATTGGG | 8 |
| 3 | HT3 | 394635 | TTCCGCCTCTCTTTG | 9 |
| 4 | HT4 | 394637 | AGTAAGTTCAGCGTA | 10 |
| 5 | HT1 + HT2 | | | |
| 6 | HT1 + HT3 | | | |
| 7 | HT1 + HT4 | | | |
| 8 | HT2 + HT3 | | | |
| 9 | HT2 + HT4 | | | |
| 10 | HT3 + HT4 | | | |

TABLE 2
Antibodies

| | Catalog # | | SEQ ID |
|---|---|---|---|
| Antibody | (Biolegend) | Barcode | NO: |
| CD56 | 362561 | TTCGCCGCATTGAGT | 11 |
| CD28 | 302961 | TGAGAACGACCCTAA | 12 |
| CD45 | 304066 | TGCAATTACCCGGAT | 13 |
| CD4 | 300565 | TGTTCCCGCTCAACT | 14 |
| IgG | 400291 | CTCCTACCTAAACTG | 15 |
| CD155 | 337637 | ATCACATCGTTGCCA | 16 |
| (PVR) | | | |
| CD29 | 303033 | GTATTCCCTCAGTCA | 17 |
| c-kit | 313247 | AGACTAATAGCTGAC | 18 |
| (CD117) | | | |
| CD20 | 302361 | TTCTGGGTCCCTAGA | 19 |
| CD16 | 302063 | AAGTTCACTCTTTGC | 20 |
| CD2 | 309233 | TACGATTTGTCAGGG | 21 |
| CD13 | 301731 | TTTCAACGCCCTTTC | 22 |
| CD25 | 302647 | TTTGTCCTGTACGCC | 23 |
| HLA-DR | 307661 | AATAGCGAGCAAGTA | 24 |
| CD30 | 333921 | TCAGGGTGTGCTGTA | 25 |
| CD10 | 312235 | CAGCCATTCATTAGG | 26 |
| CD33 | 366635 | TAACTCAGGGCCTAT | 27 |
| Kappa | 316535 | AGCTCAGCCAGTATG | 28 |
| Lambda | 316633 | CAGCCAGTAAGTCAC | 29 |
| CD14 | 367145 | CAATCAGACCTATGA | 30 |
| CD19 | 302263 | CTGGGCAATTACTCG | 31 |
| CD11b | 301357 | TGAAGGCTCATTTGT | 32 |
| CD8 | 344757 | GCGCAACTTGATGAT | 33 |
| CD3 | 300477 | CTCATTGTAACTCCT | 34 |
| NKp46 | 331939 | ACAATTTGAACAGCG | 35 |
| (CD335) | | | |
| NKG2D | 320839 | CGTGTTTGTTCCTCA | 36 |
| (CD314) | | | |
| CTLA-4 | 369629 | ATGGTTCACGTAATC | 37 |
| (CD152) | | | |
| PD-1 | 329961 | ACAGCGCCGTATTTA | 38 |
| (CD279) | | | |
| PD-L1 | 329749 | GTTGTCCGACAATAC | 39 |
| (CD274) | | | |

Split-Pool Ligation Barcoding

To prevent cells from sticking to the sides of the wells, all PCR plates and microcentrifuge tubes used were first blocked with 5% FBS in PBS for at least an hour before use.

Ligation plates were prepared ahead of time and stored until ready to use. Round 1 wells contained 12 μM barcode primer and 11 μM splint primer in water for a total volume of 10 μl. Round 2 wells contained 14 μM barcode primer and 13 μM splint primer in water for a total volume of 10 μl (Table 3). Barcoding plates were heated in a thermocycler to 95° C. for 2 minutes to anneal primers before ramping down to 20° C. at a rate of −0.1° C./s and a final hold at 4° C. until ready to use.

Twenty microliters of stained cells were aliquoted into each well of a 96 well plate for the first-round ligation, which contained the round 1 well barcode oligos and round 1 splint primer. The mixture was gently mixed by pipetting up and down several times. Ligation mix containing 1 μl T4 DNA ligase (400,000 units/ml), 5 μl 10× T4 ligase buffer, and 14 μl water was added to each well. The ligation reaction was performed in a thermocycler at 37° C. for 30 minutes. The round one splint primer was then blocked to prevent promiscuous ligation upon pooling the wells by the addition of 2.64 μl of 100 μM blocking primer, 2.5 ul of 10× ligation buffer, and 4.86 μl water to each well. The plate was then incubated in a thermocycler for 30 minutes at 37° C. to enable the blocking primer to anneal to the splint. Cells were then pooled and mixed by gentle inversion before 100 μl of T4 DNA ligase (400,000 units/ml) was added to the cell mixture and cells were redistributed into 96 round two ligation reaction plates and incubated at 37° C. for 30 minutes. Round 2 reactions were blocked with termination solution (3.3 μM blocking primer, 0.36 M EDTA; final concentrations).

TABLE 3
Primers

| | | SEQ ID |
|---|---|---|
| Sequence | Use | NO: |
| /5Phos/GAGTTCGTGCACCTAAACGTGATTCAGCATGCGGCTAC | Round 1 | 40 |
| /5Phos/GAGTTCGTGCACCTAAAACATCGTCAGCATGCGGCTAC | Round 1 | 41 |
| /5Phos/GAGTTCGTGCACCTAATGCCTAATCAGCATGCGGCTAC | Round 1 | 42 |
| /5Phos/GAGTTCGTGCACCTAAGTGGTCATCAGCATGCGGCTAC | Round 1 | 43 |
| /5Phos/GAGTTCGTGCACCTAACCACTGTTCAGCATGCGGCTAC | Round 1 | 44 |
| /5Phos/GAGTTCGTGCACCTAACATTGGCTCAGCATGCGGCTAC | Round 1 | 45 |
| /5Phos/GAGTTCGTGCACCTACAGATCTGTCAGCATGCGGCTAC | Round 1 | 46 |
| /5Phos/GAGTTCGTGCACCTACATCAAGTTCAGCATGCGGCTAC | Round 1 | 47 |
| /5Phos/GAGTTCGTGCACCTACGCTGATCTCAGCATGCGGCTAC | Round 1 | 48 |
| /5Phos/GAGTTCGTGCACCTAACAAGCTATCAGCATGCGGCTAC | Round 1 | 49 |
| /5Phos/GAGTTCGTGCACCTACTGTAGCCTCAGCATGCGGCTAC | Round 1 | 50 |
| /5Phos/GAGTTCGTGCACCTAAGTACAAGTCAGCATGCGGCTAC | Round 1 | 51 |
| /5Phos/GAGTTCGTGCACCTAAACAACCATCAGCATGCGGCTAC | Round 1 | 52 |
| /5Phos/GAGTTCGTGCACCTAAACCGAGATCAGCATGCGGCTAC | Round 1 | 53 |
| /5Phos/GAGTTCGTGCACCTAAACGCTTATCAGCATGCGGCTAC | Round 1 | 54 |
| /5Phos/GAGTTCGTGCACCTAAAGACGGATCAGCATGCGGCTAC | Round 1 | 55 |
| /5Phos/GAGTTCGTGCACCTAAAGGTACATCAGCATGCGGCTAC | Round 1 | 56 |
| /5Phos/GAGTTCGTGCACCTAACACAGAATCAGCATGCGGCTAC | Round 1 | 57 |
| /5Phos/GAGTTCGTGCACCTAACAGCAGATCAGCATGCGGCTAC | Round 1 | 58 |
| /5Phos/GAGTTCGTGCACCTAACCTCCAATCAGCATGCGGCTAC | Round 1 | 59 |
| /5Phos/GAGTTCGTGCACCTAACGCTCGATCAGCATGCGGCTAC | Round 1 | 60 |
| /5Phos/GAGTTCGTGCACCTAACGTATCATCAGCATGCGGCTAC | Round 1 | 61 |
| /5Phos/GAGTTCGTGCACCTAACTATGCATCAGCATGCGGCTAC | Round 1 | 62 |
| /5Phos/GAGTTCGTGCACCTAAGAGTCAATCAGCATGCGGCTAC | Round 1 | 63 |
| /5Phos/GAGTTCGTGCACCTAAGATCGCATCAGCATGCGGCTAC | Round 1 | 64 |
| /5Phos/GAGTTCGTGCACCTAAGCAGGAATCAGCATGCGGCTAC | Round 1 | 65 |
| /5Phos/GAGTTCGTGCACCTAAGTCACTATCAGCATGCGGCTAC | Round 1 | 66 |
| /5Phos/GAGTTCGTGCACCTAATCCTGTATCAGCATGCGGCTAC | Round 1 | 67 |
| /5Phos/GAGTTCGTGCACCTAATTGAGGATCAGCATGCGGCTAC | Round 1 | 68 |
| /5Phos/GAGTTCGTGCACCTACAACCACATCAGCATGCGGCTAC | Round 1 | 69 |
| /5Phos/GAGTTCGTGCACCTAGACTAGTATCAGCATGCGGCTAC | Round 1 | 70 |
| /5Phos/GAGTTCGTGCACCTACAATGGAATCAGCATGCGGCTAC | Round 1 | 71 |
| /5Phos/GAGTTCGTGCACCTACACTTCGATCAGCATGCGGCTAC | Round 1 | 72 |
| /5Phos/GAGTTCGTGCACCTACAGCGTTATCAGCATGCGGCTAC | Round 1 | 73 |
| /5Phos/GAGTTCGTGCACCTACATACCAATCAGCATGCGGCTAC | Round 1 | 74 |
| /5Phos/GAGTTCGTGCACCTACCAGTTCATCAGCATGCGGCTAC | Round 1 | 75 |
| /5Phos/GAGTTCGTGCACCTACCGAAGTATCAGCATGCGGCTAC | Round 1 | 76 |
| /5Phos/GAGTTCGTGCACCTACCGTGAGATCAGCATGCGGCTAC | Round 1 | 77 |
| /5Phos/GAGTTCGTGCACCTACCTCCTGATCAGCATGCGGCTAC | Round 1 | 78 |
| /5Phos/GAGTTCGTGCACCTACGAACTTATCAGCATGCGGCTAC | Round 1 | 79 |
| /5Phos/GAGTTCGTGCACCTACGACTGGATCAGCATGCGGCTAC | Round 1 | 80 |
| /5Phos/GAGTTCGTGCACCTACGCATACATCAGCATGCGGCTAC | Round 1 | 81 |
| /5Phos/GAGTTCGTGCACCTACTCAATGATCAGCATGCGGCTAC | Round 1 | 82 |
| /5Phos/GAGTTCGTGCACCTACTGAGCCATCAGCATGCGGCTAC | Round 1 | 83 |
| /5Phos/GAGTTCGTGCACCTACTGGCATATCAGCATGCGGCTAC | Round 1 | 84 |
| /5Phos/GAGTTCGTGCACCTAGAATCTGATCAGCATGCGGCTAC | Round 1 | 85 |
| /5Phos/GAGTTCGTGCACCTACAAGACTATCAGCATGCGGCTAC | Round 1 | 86 |
| /5Phos/GAGTTCGTGCACCTAGAGCTGAATCAGCATGCGGCTAC | Round 1 | 87 |
| /5Phos/GAGTTCGTGCACCTAGATAGACATCAGCATGCGGCTAC | Round 1 | 88 |
| /5Phos/GAGTTCGTGCACCTAGCCACATATCAGCATGCGGCTAC | Round 1 | 89 |
| /5Phos/GAGTTCGTGCACCTAGCGAGTAATCAGCATGCGGCTAC | Round 1 | 90 |
| /5Phos/GAGTTCGTGCACCTAGCTAACGATCAGCATGCGGCTAC | Round 1 | 91 |
| /5Phos/GAGTTCGTGCACCTAGCTCGGTATCAGCATGCGGCTAC | Round 1 | 92 |
| /5Phos/GAGTTCGTGCACCTAGGAGAACATCAGCATGCGGCTAC | Round 1 | 93 |
| /5Phos/GAGTTCGTGCACCTAGGTGCGAATCAGCATGCGGCTAC | Round 1 | 94 |
| /5Phos/GAGTTCGTGCACCTAGTACGCAATCAGCATGCGGCTAC | Round 1 | 95 |
| /5Phos/GAGTTCGTGCACCTAGTCGTAGATCAGCATGCGGCTAC | Round 1 | 96 |
| /5Phos/GAGTTCGTGCACCTAGTCTGTCATCAGCATGCGGCTAC | Round 1 | 97 |
| /5Phos/GAGTTCGTGCACCTAGTGTTCTATCAGCATGCGGCTAC | Round 1 | 98 |
| /5Phos/GAGTTCGTGCACCTATAGGATGATCAGCATGCGGCTAC | Round 1 | 99 |
| /5Phos/GAGTTCGTGCACCTATATCAGCATCAGCATGCGGCTAC | Round 1 | 100 |
| /5Phos/GAGTTCGTGCACCTATCCGTCTATCAGCATGCGGCTAC | Round 1 | 101 |
| /5Phos/GAGTTCGTGCACCTATCTTCACATCAGCATGCGGCTAC | Round 1 | 102 |
| /5Phos/GAGTTCGTGCACCTATGAAGAGATCAGCATGCGGCTAC | Round 1 | 103 |
| /5Phos/GAGTTCGTGCACCTATGGAACAATCAGCATGCGGCTAC | Round 1 | 104 |
| /5Phos/GAGTTCGTGCACCTATGGCTTCATCAGCATGCGGCTAC | Round 1 | 105 |
| /5Phos/GAGTTCGTGCACCTATGGTGGTATCAGCATGCGGCTAC | Round 1 | 106 |
| /5Phos/GAGTTCGTGCACCTATTCACGCATCAGCATGCGGCTAC | Round 1 | 107 |
| /5Phos/GAGTTCGTGCACCTAAACTCACCTCAGCATGCGGCTAC | Round 1 | 108 |
| /5Phos/GAGTTCGTGCACCTAAAGAGATCTCAGCATGCGGCTAC | Round 1 | 109 |
| /5Phos/GAGTTCGTGCACCTAAAGGACACTCAGCATGCGGCTAC | Round 1 | 110 |
| /5Phos/GAGTTCGTGCACCTAAATCCGTCTCAGCATGCGGCTAC | Round 1 | 111 |
| /5Phos/GAGTTCGTGCACCTAAATGTTGCTCAGCATGCGGCTAC | Round 1 | 112 |
| /5Phos/GAGTTCGTGCACCTAACACGACCTCAGCATGCGGCTAC | Round 1 | 113 |
| /5Phos/GAGTTCGTGCACCTAACAGATTCTCAGCATGCGGCTAC | Round 1 | 114 |
| /5Phos/GAGTTCGTGCACCTAAGATGTACTCAGCATGCGGCTAC | Round 1 | 115 |
| /5Phos/GAGTTCGTGCACCTAAGCACCTCTCAGCATGCGGCTAC | Round 1 | 116 |
| /5Phos/GAGTTCGTGCACCTAAGCCATGCTCAGCATGCGGCTAC | Round 1 | 117 |
| /5Phos/GAGTTCGTGCACCTAAGGCTAACTCAGCATGCGGCTAC | Round 1 | 118 |
| /5Phos/GAGTTCGTGCACCTAATAGCGACTCAGCATGCGGCTAC | Round 1 | 119 |
| /5Phos/GAGTTCGTGCACCTAATCATTCCTCAGCATGCGGCTAC | Round 1 | 120 |
| /5Phos/GAGTTCGTGCACCTAATTGGCTCTCAGCATGCGGCTAC | Round 1 | 121 |
| /5Phos/GAGTTCGTGCACCTACAAGGAGCTCAGCATGCGGCTAC | Round 1 | 122 |
| /5Phos/GAGTTCGTGCACCTACACCTTACTCAGCATGCGGCTAC | Round 1 | 123 |
| /5Phos/GAGTTCGTGCACCTACCATCCTCTCAGCATGCGGCTAC | Round 1 | 124 |
| /5Phos/GAGTTCGTGCACCTACCGACAACTCAGCATGCGGCTAC | Round 1 | 125 |
| /5Phos/GAGTTCGTGCACCTACCTAATCCTCAGCATGCGGCTAC | Round 1 | 126 |
| /5Phos/GAGTTCGTGCACCTACCTCTATCTCAGCATGCGGCTAC | Round 1 | 127 |
| /5Phos/GAGTTCGTGCACCTACGACACACTCAGCATGCGGCTAC | Round 1 | 128 |
| /5Phos/GAGTTCGTGCACCTACGGATTGCTCAGCATGCGGCTAC | Round 1 | 129 |
| /5Phos/GAGTTCGTGCACCTACTAAGGTCTCAGCATGCGGCTAC | Round 1 | 130 |
| /5Phos/GAGTTCGTGCACCTAGAACAGGCTCAGCATGCGGCTAC | Round 1 | 131 |
| /5Phos/GAGTTCGTGCACCTAGACAGTGCTCAGCATGCGGCTAC | Round 1 | 132 |
| /5Phos/GAGTTCGTGCACCTAGAGTTAGCTCAGCATGCGGCTAC | Round 1 | 133 |
| /5Phos/GAGTTCGTGCACCTAGATGAATCTCAGCATGCGGCTAC | Round 1 | 134 |
| /5Phos/GAGTTCGTGCACCTAGCCAAGACTCAGCATGCGGCTAC | Round 1 | 135 |
| /5Phos/GCTTTGTAGCCGGTGAACGTGATAGATCGGAAGAGCGTCGTGTAGGG | Round 2 | 136 |
| AAAGAG | | |
| /5Phos/GCTTTGTAGCCGGTGAAACATCGAGATCGGAAGAGCGTCGTGTAGGG | Round 2 | 137 |
| AAAGAG | | |
| /5Phos/GCTTTGTAGCCGGTGATGCCTAAAGATCGGAAGAGCGTCGTGTAGGG | Round 2 | 138 |
| AAAGAG | | |
| /5Phos/GCTTTGTAGCCGGTGAGTGGTCAAGATCGGAAGAGCGTCGTGTAGGG | Round 2 | 139 |
| AAAGAG | | |
| /5Phos/GCTTTGTAGCCGGTGACCACTGTAGATCGGAAGAGCGTCGTGTAGGG | Round 2 | 140 |
| AAAGAG | | |
| /5Phos/GCTTTGTAGCCGGTGACATTGGCAGATCGGAAGAGCGTCGTGTAGGG | Round 2 | 141 |
| AAAGAG | | |
| /5Phos/GCTTTGTAGCCGGTGCAGATCTGAGATCGGAAGAGCGTCGTGTAGGG | Round 2 | 142 |
| AAAGAG | | |
| /5Phos/GCTTTGTAGCCGGTGCATCAAGTAGATCGGAAGAGCGTCGTGTAGGG | Round 2 | 143 |
| AAAGAG | | |
| /5Phos/GCTTTGTAGCCGGTGCGCTGATCAGATCGGAAGAGCGTCGTGTAGGG | Round 2 | 144 |
| AAAGAG | | |
| /5Phos/GCTTTGTAGCCGGTGACAAGCTAAGATCGGAAGAGCGTCGTGTAGGG | Round 2 | 145 |
| AAAGAG | | |
| /5Phos/GCTTTGTAGCCGGTGCTGTAGCCAGATCGGAAGAGCGTCGTGTAGGG | Round 2 | 146 |
| AAAGAG | | |
| /5Phos/GCTTTGTAGCCGGTGAGTACAAGAGATCGGAAGAGCGTCGTGTAGGG | Round 2 | 147 |
| AAAGAG | | |
| /5Phos/GCTTTGTAGCCGGTGAACAACCAAGATCGGAAGAGCGTCGTGTAGGG | Round 2 | 148 |
| AAAGAG | | |
| /5Phos/GCTTTGTAGCCGGTGAACCGAGAAGATCGGAAGAGCGTCGTGTAGGG | Round 2 | 149 |
| AAAGAG | | |
| /5Phos/GCTTTGTAGCCGGTGAACGCTTAAGATCGGAAGAGCGTCGTGTAGGG | Round 2 | 150 |
| AAAGAG | | |
| /5Phos/GCTTTGTAGCCGGTGAAGACGGAAGATCGGAAGAGCGTCGTGTAGGG | Round 2 | 151 |
| AAAGAG | | |
| /5Phos/GCTTTGTAGCCGGTGAAGGTACAAGATCGGAAGAGCGTCGTGTAGGG | Round 2 | 152 |
| AAAGAG | | |
| /5Phos/GCTTTGTAGCCGGTGACACAGAAAGATCGGAAGAGCGTCGTGTAGGG | Round 2 | 153 |
| AAAGAG | | |
| /5Phos/GCTTTGTAGCCGGTGACAGCAGAAGATCGGAAGAGCGTCGTGTAGGG | Round 2 | 154 |
| AAAGAG | | |
| /5Phos/GCTTTGTAGCCGGTGACCTCCAAAGATCGGAAGAGCGTCGTGTAGGG | Round 2 | 155 |
| AAAGAG | | |
| /5Phos/GCTTTGTAGCCGGTGACGCTCGAAGATCGGAAGAGCGTCGTGTAGGG | Round 2 | 156 |
| AAAGAG | | |
| /5Phos/GCTTTGTAGCCGGTGACGTATCAAGATCGGAAGAGCGTCGTGTAGGG | Round 2 | 157 |
| AAAGAG | | |
| /5Phos/GCTTTGTAGCCGGTGACTATGCAAGATCGGAAGAGCGTCGTGTAGGG | Round 2 | 158 |
| AAAGAG | | |
| /5Phos/GCTTTGTAGCCGGTGAGAGTCAAAGATCGGAAGAGCGTCGTGTAGGG | Round 2 | 159 |
| AAAGAG | | |
| /5Phos/GCTTTGTAGCCGGTGAGATCGCAAGATCGGAAGAGCGTCGTGTAGGG | Round 2 | 160 |
| AAAGAG | | |
| /5Phos/GCTTTGTAGCCGGTGAGCAGGAAAGATCGGAAGAGCGTCGTGTAGGG | Round 2 | 161 |
| AAAGAG | | |
| /5Phos/GCTTTGTAGCCGGTGAGTCACTAAGATCGGAAGAGCGTCGTGTAGGG | Round 2 | 162 |
| AAAGAG | | |
| /5Phos/GCTTTGTAGCCGGTGATCCTGTAAGATCGGAAGAGCGTCGTGTAGGG | Round 2 | 163 |
| AAAGAG | | |
| /5Phos/GCTTTGTAGCCGGTGATTGAGGAAGATCGGAAGAGCGTCGTGTAGGG | Round 2 | 164 |
| AAAGAG | | |
| /5Phos/GCTTTGTAGCCGGTGCAACCACAAGATCGGAAGAGCGTCGTGTAGGG | Round 2 | 165 |
| AAAGAG | | |
| /5Phos/GCTTTGTAGCCGGTGGACTAGTAAGATCGGAAGAGCGTCGTGTAGGG | Round 2 | 166 |
| AAAGAG | | |
| /5Phos/GCTTTGTAGCCGGTGCAATGGAAAGATCGGAAGAGCGTCGTGTAGGG | Round 2 | 167 |
| AAAGAG | | |
| /5Phos/GCTTTGTAGCCGGTGCACTTCGAAGATCGGAAGAGCGTCGTGTAGGG | Round 2 | 168 |
| AAAGAG | | |
| /5Phos/GCTTTGTAGCCGGTGCAGCGTTAAGATCGGAAGAGCGTCGTGTAGGG | Round 2 | 169 |
| AAAGAG | | |
| /5Phos/GCTTTGTAGCCGGTGCATACCAAAGATCGGAAGAGCGTCGTGTAGGG | Round 2 | 170 |
| AAAGAG | | |
| /5Phos/GCTTTGTAGCCGGTGCCAGTTCAAGATCGGAAGAGCGTCGTGTAGGG | Round 2 | 171 |
| AAAGAG | | |
| /5Phos/GCTTTGTAGCCGGTGCCGAAGTAAGATCGGAAGAGCGTCGTGTAGGG | Round 2 | 172 |
| AAAGAG | | |
| /5Phos/GCTTTGTAGCCGGTGCCGTGAGAAGATCGGAAGAGCGTCGTGTAGGG | Round 2 | 173 |
| AAAGAG | | |
| /5Phos/GCTTTGTAGCCGGTGCCTCCTGAAGATCGGAAGAGCGTCGTGTAGGG | Round 2 | 174 |
| AAAGAG | | |
| /5Phos/GCTTTGTAGCCGGTGCGAACTTAAGATCGGAAGAGCGTCGTGTAGGG | Round 2 | 175 |
| AAAGAG | | |
| /5Phos/GCTTTGTAGCCGGTGCGACTGGAAGATCGGAAGAGCGTCGTGTAGGG | Round 2 | 176 |
| AAAGAG | | |
| /5Phos/GCTTTGTAGCCGGTGCGCATACAAGATCGGAAGAGCGTCGTGTAGGG | Round 2 | 177 |
| AAAGAG | | |
| /5Phos/GCTTTGTAGCCGGTGCTCAATGAAGATCGGAAGAGCGTCGTGTAGGG | Round 2 | 178 |
| AAAGAG | | |
| /5Phos/GCTTTGTAGCCGGTGCTGAGCCAAGATCGGAAGAGCGTCGTGTAGGG | Round 2 | 179 |
| AAAGAG | | |
| /5Phos/GCTTTGTAGCCGGTGCTGGCATAAGATCGGAAGAGCGTCGTGTAGGG | Round 2 | 180 |
| AAAGAG | | |
| /5Phos/GCTTTGTAGCCGGTGGAATCTGAAGATCGGAAGAGCGTCGTGTAGGG | Round 2 | 181 |
| AAAGAG | | |
| /5Phos/GCTTTGTAGCCGGTGCAAGACTAAGATCGGAAGAGCGTCGTGTAGGG | Round 2 | 182 |
| AAAGAG | | |
| /5Phos/GCTTTGTAGCCGGTGGAGCTGAAAGATCGGAAGAGCGTCGTGTAGGG | Round 2 | 183 |
| AAAGAG | | |
| /5Phos/GCTTTGTAGCCGGTGGATAGACAAGATCGGAAGAGCGTCGTGTAGGG | Round 2 | 184 |
| AAAGAG | | |
| /5Phos/GCTTTGTAGCCGGTGGCCACATAAGATCGGAAGAGCGTCGTGTAGGG | Round 2 | 185 |
| AAAGAG | | |
| /5Phos/GCTTTGTAGCCGGTGGCGAGTAAAGATCGGAAGAGCGTCGTGTAGGG | Round 2 | 186 |
| AAAGAG | | |
| /5Phos/GCTTTGTAGCCGGTGGCTAACGAAGATCGGAAGAGCGTCGTGTAGGG | Round 2 | 187 |
| AAAGAG | | |
| /5Phos/GCTTTGTAGCCGGTGGCTCGGTAAGATCGGAAGAGCGTCGTGTAGGG | Round 2 | 188 |
| AAAGAG | | |
| /5Phos/GCTTTGTAGCCGGTGGGAGAACAAGATCGGAAGAGCGTCGTGTAGGG | Round 2 | 189 |
| AAAGAG | | |
| /5Phos/GCTTTGTAGCCGGTGGGTGCGAAAGATCGGAAGAGCGTCGTGTAGGG | Round 2 | 190 |
| AAAGAG | | |
| /5Phos/GCTTTGTAGCCGGTGGTACGCAAAGATCGGAAGAGCGTCGTGTAGGG | Round 2 | 191 |
| AAAGAG | | |
| /5Phos/GCTTTGTAGCCGGTGGTCGTAGAAGATCGGAAGAGCGTCGTGTAGGG | Round 2 | 192 |
| AAAGAG | | |
| /5Phos/GCTTTGTAGCCGGTGGTCTGTCAAGATCGGAAGAGCGTCGTGTAGGG | Round 2 | 193 |
| AAAGAG | | |
| /5Phos/GCTTTGTAGCCGGTGGTGTTCTAAGATCGGAAGAGCGTCGTGTAGGG | Round 2 | 194 |
| AAAGAG | | |
| /5Phos/GCTTTGTAGCCGGTGTAGGATGAAGATCGGAAGAGCGTCGTGTAGGG | Round 2 | 195 |
| AAAGAG | | |
| /5Phos/GCTTTGTAGCCGGTGTATCAGCAAGATCGGAAGAGCGTCGTGTAGGG | Round 2 | 196 |
| AAAGAG | | |
| /5Phos/GCTTTGTAGCCGGTGTCCGTCTAAGATCGGAAGAGCGTCGTGTAGGG | Round 2 | 197 |
| AAAGAG | | |
| /5Phos/GCTTTGTAGCCGGTGTCTTCACAAGATCGGAAGAGCGTCGTGTAGGG | Round 2 | 198 |
| AAAGAG | | |
| /5Phos/GCTTTGTAGCCGGTGTGAAGAGAAGATCGGAAGAGCGTCGTGTAGGG | Round 2 | 199 |
| AAAGAG | | |
| /5Phos/GCTTTGTAGCCGGTGTGGAACAAAGATCGGAAGAGCGTCGTGTAGGG | Round 2 | 200 |
| AAAGAG | | |
| /5Phos/GCTTTGTAGCCGGTGTGGCTTCAAGATCGGAAGAGCGTCGTGTAGGG | Round 2 | 201 |
| AAAGAG | | |
| /5Phos/GCTTTGTAGCCGGTGTGGTGGTAAGATCGGAAGAGCGTCGTGTAGGG | Round 2 | 202 |
| AAAGAG | | |
| /5Phos/GCTTTGTAGCCGGTGTTCACGCAAGATCGGAAGAGCGTCGTGTAGGG | Round 2 | 203 |
| AAAGAG | | |
| /5Phos/GCTTTGTAGCCGGTGAACTCACCAGATCGGAAGAGCGTCGTGTAGGG | Round 2 | 204 |
| AAAGAG | | |
| /5Phos/GCTTTGTAGCCGGTGAAGAGATCAGATCGGAAGAGCGTCGTGTAGGG | Round 2 | 205 |
| AAAGAG | | |
| /5Phos/GCTTTGTAGCCGGTGAAGGACACAGATCGGAAGAGCGTCGTGTAGGG | Round 2 | 206 |
| AAAGAG | | |
| /5Phos/GCTTTGTAGCCGGTGAATCCGTCAGATCGGAAGAGCGTCGTGTAGGG | Round 2 | 207 |
| AAAGAG | | |
| /5Phos/GCTTTGTAGCCGGTGAATGTTGCAGATCGGAAGAGCGTCGTGTAGGG | Round 2 | 208 |
| AAAGAG | | |
| /5Phos/GCTTTGTAGCCGGTGACACGACCAGATCGGAAGAGCGTCGTGTAGGG | Round 2 | 209 |
| AAAGAG | | |
| /5Phos/GCTTTGTAGCCGGTGACAGATTCAGATCGGAAGAGCGTCGTGTAGGG | Round 2 | 210 |
| AAAGAG | | |
| /5Phos/GCTTTGTAGCCGGTGAGATGTACAGATCGGAAGAGCGTCGTGTAGGG | Round 2 | 211 |
| AAAGAG | | |
| /5Phos/GCTTTGTAGCCGGTGAGCACCTCAGATCGGAAGAGCGTCGTGTAGGG | Round 2 | 212 |
| AAAGAG | | |
| /5Phos/GCTTTGTAGCCGGTGAGCCATGCAGATCGGAAGAGCGTCGTGTAGGG | Round 2 | 213 |
| AAAGAG | | |
| /5Phos/GCTTTGTAGCCGGTGAGGCTAACAGATCGGAAGAGCGTCGTGTAGGG | Round 2 | 214 |
| AAAGAG | | |
| /5Phos/GCTTTGTAGCCGGTGATAGCGACAGATCGGAAGAGCGTCGTGTAGGG | Round 2 | 215 |
| AAAGAG | | |
| /5Phos/GCTTTGTAGCCGGTGATCATTCCAGATCGGAAGAGCGTCGTGTAGGG | Round 2 | 216 |
| AAAGAG | | |
| /5Phos/GCTTTGTAGCCGGTGATTGGCTCAGATCGGAAGAGCGTCGTGTAGGG | Round 2 | 217 |
| AAAGAG | | |
| /5Phos/GCTTTGTAGCCGGTGCAAGGAGCAGATCGGAAGAGCGTCGTGTAGGG | Round 2 | 218 |
| AAAGAG | | |
| /5Phos/GCTTTGTAGCCGGTGCACCTTACAGATCGGAAGAGCGTCGTGTAGGG | Round 2 | 219 |
| AAAGAG | | |
| /5Phos/GCTTTGTAGCCGGTGCCATCCTCAGATCGGAAGAGCGTCGTGTAGGG | Round 2 | 220 |
| AAAGAG | | |
| /5Phos/GCTTTGTAGCCGGTGCCGACAACAGATCGGAAGAGCGTCGTGTAGGG | Round 2 | 221 |
| AAAGAG | | |
| /5Phos/GCTTTGTAGCCGGTGCCTAATCCAGATCGGAAGAGCGTCGTGTAGGG | Round 2 | 222 |
| AAAGAG | | |
| /5Phos/GCTTTGTAGCCGGTGCCTCTATCAGATCGGAAGAGCGTCGTGTAGGG | Round 2 | 223 |
| AAAGAG | | |
| /5Phos/GCTTTGTAGCCGGTGCGACACACAGATCGGAAGAGCGTCGTGTAGGG | Round 2 | 224 |
| AAAGAG | | |
| /5Phos/GCTTTGTAGCCGGTGCGGATTGCAGATCGGAAGAGCGTCGTGTAGGG | Round 2 | 225 |
| AAAGAG | | |
| /5Phos/GCTTTGTAGCCGGTGCTAAGGTCAGATCGGAAGAGCGTCGTGTAGGG | Round 2 | 226 |
| AAAGAG | | |
| /5Phos/GCTTTGTAGCCGGTGGAACAGGCAGATCGGAAGAGCGTCGTGTAGGG | Round 2 | 227 |
| AAAGAG | | |
| /5Phos/GCTTTGTAGCCGGTGGACAGTGCAGATCGGAAGAGCGTCGTGTAGGG | Round 2 | 228 |
| AAAGAG | | |
| /5Phos/GCTTTGTAGCCGGTGGAGTTAGCAGATCGGAAGAGCGTCGTGTAGGG | Round 2 | 229 |
| AAAGAG | | |
| /5Phos/GCTTTGTAGCCGGTGGATGAATCAGATCGGAAGAGCGTCGTGTAGGG | Round 2 | 230 |
| AAAGAG | | |
| /5Phos/GCTTTGTAGCCGGTGGCCAAGACAGATCGGAAGAGCGTCGTGTAGGG | Round 2 | 231 |
| AAAGAG | | |
| TAGGTGCACGAACTCTTGCTAGGACCGGC | Round 1 | 232 |
| | splint | |
| | primer | |
| CACCGGCTACAAAGCGTAGCCGCATGCTGA | Round 2 | 233 |
| | splint | |
| | primer | |
| GCCGGTCCTAGCAAGAGTTCGTGCACCTA | Round 1 | 234 |
| | blocking | |
| | primer | |
| TCAGCATGCGGCTACGCTTTGTAGCCGGTG | Round 2 | 235 |
| | blocking | |
| | primer | |

Cells were pooled after round 2 blocking and counted on a hemocytometer via light microscopy. Cells were diluted to an appropriate final concentration before final round barcoding by PCR. The dilution factor is calculated such that 1 μl of input volume contains enough cells per well such that the total number of cells profiled across all wells does not exceed 2% of the total barcode capacity (to avoid barcode collision doublets). A single round of PCR was performed at 14 cycles. DNA fragments were cleaned up for sequencing through two rounds of bead-based cleanup (at a 0.9 ratio of beads to DNA) according to the manufacturer's protocol (AMPure XP).

Sequencing, Demultiplexing, and Bioinformatics

Paired end sequencing was set up to sequence 100 bases from the 5′ end and 50 bases from the 3′ end. Samples were sequenced on NextSeq 500/550 and sequences were demultiplexed in Illumina BaseSpace according to the default quality control settings before subsequent processing.

UMI-tools was used to extract well-barcodes from the 5′ read and antibody barcodes along with UMIs from the 3′ read. Count matrices are generated using custom python scripts (Table 4).

TABLE 4
Plate Map
Plate 1

| | | | | | | | | | | | |
|---|---|---|---|---|---|---|---|---|---|---|---|
| A170 | A168 | A160 | A146 | A143 | A140 | A125 | A124 | A181 | A158 | A102 | A92 |
| A688 | A630 | A624 | A686 | A683 | A663 | A618 | A603 | A693 | A680 | A669 | A667 |
| A151 | A123 | A118 | A114 | A108 | A104 | A165 | A142 | A162 | A121 | A179 | A163 |
| A665 | A664 | A639 | A612 | A608 | A607 | A606 | A605 | A602 | A595 | A684 | A681 |
| A155 | A148 | A111 | A194 | A193 | A190 | A188 | A187 | A182 | A172 | A169 | A152 |
| A674 | A634 | A627 | A623 | A677 | A648 | A643 | A642 | A638 | A692 | A682 | A662 |
| A144 | A133 | A115 | A109 | A105 | A195 | A177 | A171 | A164 | A157 | A150 | A137 |
| A661 | A660 | A666 | A654 | A649 | A637 | A633 | A620 | A599 | A676 | A629 | A626 |

Plate 2

| | | | | | | | | | | | |
|---|---|---|---|---|---|---|---|---|---|---|---|
| A136 | A135 | A134 | A149 | A97 | A95 | A173 | A159 | A156 | A147 | A139 | A132 |
| A616 | A700 | A699 | A694 | A691 | A687 | A679 | A670 | A659 | A651 | A650 | A647 |
| A130 | A107 | A99 | A191 | A178 | A138 | A119 | A106 | A103 | A113 | A184 | A183 |
| A645 | A641 | A628 | A614 | A613 | A689 | A685 | A678 | A675 | A657 | A615 | A690 |
| A180 | A176 | A175 | A117 | A116 | A110 | A185 | A166 | A153 | A141 | A122 | A174 |
| A671 | A656 | A655 | A653 | A631 | A611 | A610 | A673 | A668 | A658 | A652 | A632 |
| A161 | A131 | A129 | A186 | A128 | A127 | A126 | A120 | A167 | A154 | A145 | A112 |
| A625 | A621 | A617 | A697 | A672 | A646 | A644 | A640 | A636 | A635 | A622 | A619 |

Counts are transformed via center-log-ratio transformation where the CLR scores for cell are defined as:

$$C L R ( x ) = [ \frac{{x}_{1}}{g ( x )} , … , \frac{{x}_{n}}{g ( x )} ] \qquad Formula 1$$

where g is the geometric mean of the protein counts for the cell. Dimension reduction is performed via TSNE on the CLR scores using a PCA initialization.

Data Availability

The raw sequencing data of experiments are available under NCBI BioProject accession no. PRJNA750440.

Results: A quantitative and highly modular method for high-dimensional proteomic profiling

PSP-seq leverages a split-pool method to uniquely identify protein expression levels on single cells (Vitak et al., (2017). Sequencing thousands of single-cell genomes with combinatorial indexing. Nature Methods, 14(3), 302-308; Rosenberg et al., (2018). Single-cell profiling of the developing mouse brain and spinal cord with split-pool barcoding. Science, 360(6385), 176-182; O'Huallachain et al., 2020; Cao et al., (2017). Comprehensive single-cell transcriptional profiling of a multicellular organism. Science, 357(6352), 661-667). The method uses a panel of DNA-barcoded antibodies, which are incubated with a sample of interest, in order to quantify expression levels of proteins on single cells. After washing away unbound antibodies, cells are randomly deposited into a series of wells (FIG. 9A step i). Within each well, a universal splint primer is used to facilitate the ligation of a short DNA oligo containing a unique well barcode to the 3′ end of all DNA-barcoded antibodies within the well (FIG. 9A step ii). After first round ligation, a short oligo, complementary to the splint primer, is incubated with the cell mixture to block inappropriate ligation during future steps. Subsequently, the cells are pooled, gently mixed, and randomly deposited into a new set of wells, into which a second unique well barcode is added, using a different splint primer. After the second round of ligation, the split primer is once again blocked and the cells are pooled and deposited into a new plate. In this final plate, PCR is used to amplify and append a third unique well barcode (FIG. 9A step iii) onto the DNA oligonucleotide coupled to the antibody. The resulting amplicons which are analyzed through NGS each contain an antibody barcode and 3 well barcodes (2 from ligation and 1 from PCR). For each amplicon, the antibody barcode is used to understand the antigen the antibody was bound to and the well barcodes are used to decipher which unique cell the antibody was bound to, as all antibodies interacting with the same cell would be expected to be labeled with the same 3 well barcodes. Collision rates are kept low by ensuring that only a small fraction of the total tripartite well-barcode combination is used (as barcode doublet rates can be thought of as a Poisson process). By grouping together all antibody sequences with the same trio of well barcodes, a comprehensive profile of the proteins present on each single cell can be determined. For PSP-seq, experimental capacity is limited only by the number of wells in each round of barcoding and by the desired number of rounds of split-pool.

In order to benchmark PSP-seq, we initially performed a mixed cell experiment with two distinct cell lines, Jurkat E6.1 (Jurkat) and HEK293T (FIG. 10). The two cell lines were cultured separately and combined at a 1:1 ratio before staining with a carefully selected panel of antibodies (CD56, CD155, CD29, CD4, CD45, CD28, and isotype control) that were designed to enable us to readily distinguish between the two human cell lines (FIG. 11A). Using CD4 and CD56 as unique identifying markers for Jurkat and HEK293T cells, respectively, PSP-seq correctly identified individual cells as being either of one cell line or another with only a small proportion of cells (2.5%) showing both CD4 and CD56 (FIG. 11B). The small number of single cells containing both CD4 and CD56 markers likely represent “collisions” in this experiment (i.e. pairs of cells that received the same trio of well barcodes) and is commensurate with doublets detected in standard single-cell RNA sequencing experiments (Wolock, S. L., et al. (2019). Scrublet: Computational Identification of Cell Doublets in Single-Cell Transcriptomic Data. Cell Systems, 8(4), 281-291), as well as the theoretical figure predicted by Poisson loading; a barcode utilization rate of 2% in this experiment is predicted to give a doublet rate of −1%.

Jurkat cells spiked in at 5% of the initial pool, stained only with a hashtag (HT2, against universally expressed proteins CD298 and (32 microglobulin) DNA-barcoded antibodies, were included in this mixed cell line experiment. These cells were designed to serve as controls, to quantify the rate of barcode swapping during PCR amplification and NGS, along with inappropriate signal caused by antibodies disassociating from their initial target cell and binding a new one during the rounds of split-pooling (FIG. 10). These HT2-stained controls showed no CD56 or CD4 antibody signal (FIG. 11B), suggesting that the signals we observe from PSP-seq are from genuine interactions between antibodies and their target cell. As a control for efficient blocking post-ligation, Jurkat cells stained only with HT3 (a hashtag antibody with a different antibody barcode) DNA-barcoded antibodies, were spiked in at 5% of the total cell count after the first round ligation (FIG. 10). These cells were designed to assay the efficiency at which we blocked the first round split oligonucleotide, before pooling the cells. If the blocking of the round 1 split oligonucleotide was highly efficient we would expect no HT3-stained cells to be detected in the final analysis, given that they never received the first round of ligation which is required to be a substrate for the second round. As anticipated, no HT3-stained cells (Materials and Methods, en supra) were detected in sequencing. These data indicate that barcodes are only ligated within their appropriate well and are not being inappropriately ligated to the incorrect cell during our subsequent rounds of pooling and further manipulation.

To establish the quantitative nature of PSP-seq, we compared the center-log-ratio (CLR)-transformed scores of antibody counts from PSP-seq to the current gold standard for protein quantification, flow cytometry (Aitchison, J. (1982). The Statistical Analysis of Compositional Data. Journal of the Royal Statistical Society. Series B, 44(2), 139-177; Stoeckius et al., 2017). In these experiments, Jurkat cells were co-stained with an antibody cocktail containing both CD4 DNA-barcoded antibodies and CD4-FITC antibodies. Cells were first sorted through fluorescent activated cell sorting (FACS) based on fluorescent CD4-FITC levels. High, medium, and low CD4-FITC cell populations were then stained with hashtag antibodies to mark their FACS sorted population identity before processing via PSP-seq (FIG. 11C). CLR-transformed scores of CD4 for each sorted population were compared against FACS measurements to show that PSP-seq can quantitatively differentiate protein expression levels in single cells (FIG. 11D-11E).

Detailed benchmarking of this method shows that sequencing coverage per cell is much lower than traditional single-cell RNA-seq, where 105-106 reads per cell are targeted (FIG. 12A-12D) (Pollen, A. A., et al (2014). Low-coverage single-cell mRNA sequencing reveals cellular heterogeneity and activated signaling pathways in developing cerebral cortex. Nature Biotechnology, 32(10), 1053-1058). However, due to the over three orders of magnitude abundance of proteins over RNA, coupled with the lower dimensionality of the sample proteome space, this difference does not compromise the ability to accurately identify different cell types (FIG. 12C-12D). Even cells with as few as 100 UMIs detected could be assayed and identified.

PSP-Seq can be Used to Quantify Protein Expression in Complex Samples

In cases where multiple samples need to be processed in parallel, DNA-barcoded hashtag antibodies, against universally expressed proteins CD298 and (32 microglobulin, are used to uniquely tag each sample before samples are pooled and stained with a panel of DNA-barcoded antibodies against proteins of interest. In this experiment, we use unique combinations of four hashtag antibodies to uniquely label 10 patient samples (Table 1). After staining, these cells are then ready for subsequent ligation and PCR steps.

Clinical flow cytometry demands high fidelity due to its critical role in the diagnostic process. We provide initial evidence that not only does PSP-seq live up to such data standards, but that it offers advantages by allowing for use of a single antibody panel to simultaneously quantify all antigens of interest, along with multiplexed processing of samples. To demonstrate the potential of PSP-seq, we conducted PSP-seq on 10 varied deidentified patient samples from the Columbia University Immunogenetics and Cellular Immunology Lab. Individual blood and bone marrow samples were uniquely labeled with hashtag antibodies before isolation of peripheral blood mononuclear cells (PBMCs) with Ficoll-Paque reagent. Next, hashtag-labeled PBMCs from all 10 samples were then pooled and collectively stained with a panel of 29 DNA-barcoded antibodies. Cells were taken through two rounds of split-pool ligation with a third round of barcoding performed through PCR. A total of over thirty thousand cells were profiled (FIG. 13A-13B).

We were able to clearly distinguish cells originating from each of the ten patient samples. The information obtained from PSP-seq largely corroborated clinical testing diagnosis, which included but was not limited to flow cytometry (See Table 5).

TABLE 5

| | Diagnosis from clinical tests and | |
|---|---|---|
| Patient | flow cytometry | Corroborating evidence from PSP-seq |
| 1 | Chronic Lymphocytic Leukemia | Kappa-lambda imbalance in B-cells |
| | (CLL) | (Kappa expansion) |
| 2 | Chronic Lymphocytic Leukemia | Kappa-lambda imbalance in B-cells |
| | (CLL) | (Lambda expansion) |
| 3 | Chronic Lymphocytic Leukemia | Kappa-lambda imbalance in B-cells |
| | (CLL) | (Lambda expansion) |
| 4 | Elevated CD4+ CD25+ (low | N/A due to low viability/cellularity |
| | viability/cellularity) | |
| 5 | Post-transplant Lymphoproliferative | N/A due to low viability/cellularity |
| | disorder (PTLD) and Non-Hodgkin | |
| | Lymphoma with targeted therapies | |
| | (low viability/cellularity) | |
| 6 | Chronic Lymphocytic Leukemia | Kappa-lambda imbalance in B-cells |
| | (CLL) | (Kappa expansion) |
| 7 | No pathology (remission monitoring) | Everything within normal range |
| 8 | Anemiathat | Everything within normal range; no |
| | | diagnostic cell surface markers |
| 9 | Adult T-cell lymphoma (ATLL) | CD4+:CD8+ imbalance; elevated |
| | | CD25+, CTLA-4 |
| 10 | B-cell Acute Lymphblastic Leukemia | Proliferation of CD19+CD20−CD45-cells |
| | (B-ALL) | consistent with expansion of pre-B-I |
| | | cells and B-ALL |

Patient diagnosis and monitoring information. Diagnostic information obtained from clinical testing including flow cytometry was compared against evidence obtained from PSP-seq to corroborate diagnostic takeaways.

Of these, we focus in on two in particular which show intriguing phenotypes to showcase the ability of PSP-seq to identify common and pathologic cellular subsets. Analysis of sample 1 focuses on the expanded B-cell population, characterized by CD19+CD3− (FIG. 15A). A comparison of lambda to kappa light chain ratio indicates an abnormal kappa light chain bias, suggestive of a patient with a B-cell chronic lymphocytic leukemia (B-CLL) (FIG. 15B). A normal kappa-lambda ratio ranges from 0.26 to 1.65 (Rajkumar, S. V., et al (2005). Serum free light chain ratio is an independent risk factor for progression in monoclonal gammopathy of undetermined significance. Blood, 106(3), 812-817). These signatures and measured expression levels reflect flow cytometry measurements of the same sample (FIG. 15A-15D). Interestingly, the T-cell marker CD8, which was not captured in the original flow panel, shows moderate expression on a subset of CD19+CD20+ cells (FIG. 15A). Though this signature has been reported previously in several cases of CLL, there is little consensus about its prognostic implications (Carulli, G., et al (2009). Aberrant Expression of CD8 in B-Cell Non-Hodgkin Lymphoma: A Multicenter Study of 951 Bone Marrow Samples With Lymphomatous Infiltration. American Journal of Clinical Pathology, 132(2), 186-190); Islam, A., et al (2000). CD8 expression on B cells in chronic lymphocytic leukemia: a case report and review of the literature. Arch Pathol Lab Med, 124(9), 1361-3). Because of the low number of cases that show this phenotype and lack of consensus about its biological implications, diagnostic panels are not designed to pick up this combination of markers. By using a PSP-seq setup, rare cell phenotypes such as these can be comprehensively examined without additional effort, to generate new knowledge and drive the field forward.

Scrutiny of sample 9 shows an expanded T-cell population. A comparison of CD3+CD4+ versus CD3+CD8+ cells reveals a pathological imbalance between CD4+ and CD8+ T-cells (FIG. 17A). A closer examination of the CD4+ cell population reveals cells with an irregularly elevated level of PD1 and CD25 consistent with the diagnosis of adult T-cell lymphoma (ATLL) originating from peripheral regulatory T-cells (FIG. 17B-17C) (Yano, H., et al (2007). Regulatory T-cell function of adult T-cell leukemia/lymphoma cells. International Journal of Cancer, 120(9), 2053-2057; Kozako, T., et al (2009). PD-1/PD-L1 expression in human T-cell leukemia virus type 1 carriers and adult T-cell leukemia/lymphoma patients. Lymphoma, 23(2), 375-382). These signatures are commensurate with the flow cytometric profiles generated for the sample (FIG. 17D). Further analysis of the PSP-seq data revealed a subset of ATLL cells with moderate CTLA-4 expression, which is not commonly observed in ATLL and was not assayed with the original flow panel (Shimauchi, T., et al (2007). Adult T-cell leukemia/lymphoma cells from blood and skin tumors express cytotoxic T lymphocyte-associated antigen-4 and Foxp3 but lack suppressor activity toward autologous CD8+ T cells. Cancer Science, 99(1), 98-106) (FIG. 17C). The significance of elevated CTLA-4 levels within ATLL remains unknown, although in other T cell malignancies increased amounts of CTLA-4 correlates with more advanced disease (Korman, A. J., et al (2006). Checkpoint Blockade in Cancer Immunotherapy. Advances in Immunology, 90, 297-339; Wong, H. K., et al (2006). Increased expression of CTLA-4 in malignant T-cells from patients with mycosis fungoides—cutaneous T cell lymphoma. Journal of Investigative Dermatology, 126(1), 212-219).

Discussion

PSP-seq enables multiplex quantification of expression levels of dozens of cell surface proteins simultaneously on individual cells through a sequencing-based cytometry technique. PSP-seq relies on having DNA-barcoded antibodies to label proteins of interest, before the cells are passed through a series of two ligation-based barcoding steps and a final PCR barcoding stage. Multiplexed analysis of samples from parallel experiments is easily accomplished by pre-hashing the samples. Thus, we showed that 10 clinical samples of blood/bone marrow from clinical patient samples could be multiplexed and their flow cytometric diagnosis confirmed using PSP-seq.

PSP-seq can be scaled to increase the number of cells assayed in two ways; first the number of barcoding wells at each stage can be increased, second the number of barcoding rounds can be expanded (an almost two order of magnitude increase in throughput can be achieved by one additional round of 96-well barcoding). Increasing the number of barcoding rounds requires small changes in primer design but can be seamlessly accommodated in the protocol without significant loss of efficiency. Thus, PSP-seq allows for a cost-effective and versatile way to profile dozens of proteins on tens of thousands of cells. As presented, the cost per cell is calculated to be ˜$0.05/cell (Table 6). This is driven largely by the cost of sequencing and price of DNA-barcoded antibodies. As sequencing technologies are widely adopted and costs have exponentially decreased over the past decade, this technology will likely become increasingly affordable over time. Furthermore, the recent interest in protein detection via DNA-barcoded antibodies has seen an increase in availability of DNA-barcoded antibodies and a reduction in their cost, making it possible to construct evermore diverse panels.

TABLE 6
Reagents and Cost

| | | | Quantity | Cost (per |
|---|---|---|---|---|
| Reagent | Vendor (Catalog #) | Cost (total) | required | experiment) |
| Ficoll-Paque | GE Healthcare | 217 (600 mL) | 20 mL | 7.23 |
| | (17-1440-02) | | | |
| Q5 Polymerase | New England | 360 (500 units) | 100 units | 72 |
| | Biolabs (M0491L) | | | |
| T4 DNA ligase | New England | 208 (100,000 | 80000 | 166.4 |
| | Biolabs (M0202L) | units) | units | |
| 96-well plates | USA Scientific | 24 (box of 10) | 3 | 7.2 |
| | (1402-9200) | | | |
| DNA barcoded | Biolegend (See | 341 each × 4 | 1 ug each | 136.4 |
| hashtag | supplementary | antibodies (10 ug | antibody | |
| antibodies | information for cat #) | each) = 1364 | | |
| DNA barcoded | Biolegend (See | 243 each × 29 | 1 ug each | 704.7 |
| antibodies | supplementary | antibodies (10 ug | antibody | |
| | information for cat #) | each) = 7047 | | |
| Human BD Fc | BD Pharmingen | 155 (0.25 mg) | 2.5 ug | 1.55 |
| block | (564220) | | | |
| Sequencing kit | Illumina/Columbia | 2939 (400M | 120M | 881.7 |
| (Paired end | University Genome | reads) | reads | |
| 150 bp); | Center | | | |
| Machine cost | | | | |
| Primers | | | | |
| Barcode | IDT | 2289.31 (4 nM) | 0.13 nM | 74.4 |
| primers | | | | |
| Splint oligos | IDT | 15 (100 nM) | 12 uM | 1.8 |
| Blocking oligos | IDT | 14 (100 nM) | 26.4 nM | 3.7 |
| Total | | | | 2057.08 |
| Number of | | | | |
| Cells | | | | |
| 40,000 | | | | |
| Cost per cell | | | | 0.051427 |
| Common | | | | |
| Laboratory | | | | |
| Reagents | Vendor (Catalog #) | | | |
| Bovine Serum | Sigma Aldrich | | | |
| Albumin (BSA) | (A9418-50G) | | | |
| 4% PFA in PBS | Alfa Aesar (J61899) | | | |
| SPRIselect | Beckman Coulter | | | |
| | (B23318) | | | |

We demonstrate that PSP-seq can be used on complex patient samples. Our cell staining workflows require little modification between what is currently used for flow cytometry making it easily adaptable and familiar to most groups. Though studies have previously showcased the potential using a split-pool method to profile protein expression (O'Huallachain et al., (2020). Ultra-high throughput single-cell analysis of proteins and RNAs by split-pool synthesis. Communications Biology, 3(1), 213; Hwang et al., 2020), we expand on this principle, demonstrating that it can be used on human clinical material with performance on par with the gold standard clinical diagnostic, flow cytometry. We show that by using a minimal panel of commercially available DNA-barcoded antibodies, we are able to detect both canonical and pathological cell types. By using PSP-seq, we gain the ability to compare expression levels of multiple proteins directly on the same cell, to gain a more comprehensive understanding of cell state and derive novel biological insight. In future iterations of the technology, we anticipate that PSP-seq can be expanded to the detection of intracellular targets using common permeabilization protocols.

While the current ecosystem of single-cell-omics has seen an explosion of development in the sequencing technology space, much of the focus has been on quantifying RNA abundances. Studies have documented that correlations between bulk RNA and protein levels to be modest in many circumstances, with estimates ranging between R-0.4-0.9 (Gry et al., (2009). Correlations between RNA and protein expression profiles in 23 human cell lines. BMC Genomics, 10(1), 365; Brion et al., (2020). Simultaneous quantification of mRNA and protein in single cells reveals post-transcriptional effects of genetic variation. eLife; Koussounadis et al., (2015). Relationship between differentially expressed mRNA and mRNA-protein correlations in a xenograft model system. Scientific Reports, 5(1), 10775). On the individual cell level, this moderate correlation completely disappears, with evidence showing little to no relationship between RNA and protein levels detected in single cells (Stoeckius et al., 2017; Taniguchi et al., (2010). Quantifying E. coli Proteome and Transcriptome with Single-Molecule Sensitivity in Single Cells. Science, 329(5991), 533-538), further cementing the importance of directly probing and assaying protein levels when trying to paint an accurate picture of cell state. PSP-seq offers an affordable, rigorous, and quantitative method to perform proteomic profiling with minimal additional equipment or technical expertise outside of that available within most modern molecular biology laboratories, opening the door to easier protein-forward discoveries and diagnostics.

The premise of PSP-seq hinges on being able to access and label nucleic acid substrates through ligation, which makes it an easy tool to extend and repurpose. While it may seem that an obvious first expansion of this technique would be to parallelize it with more traditional single-cell RNA sequencing techniques to profile the transcriptome in an unbiased way, previous work points to the low efficiency of split-pool barcoding for unbiased RNA profiling making it a poor candidate for tandem multiplexing with PSP-seq (Ding et al., (2020). Systematic comparison of single-cell and single-nucleus RNA-sequencing methods. Nature Biotechnology, 38(6), 737-746). A more promising extension of PSP-seq would be to selectively capture nucleic acids of interest (Behbehani et al., 2012). To capture RNA, an initial reverse transcription with a targeted capture primer followed by a second strand synthesis would be necessary to form a stable DNA scaffold for the ligation barcoding reactions. Subsequent rounds of barcoding would ligate onto the 3′ end of the synthesized strand. Targeted sequencing would also make it possible to use PSP-seq to multiplex CRISPR screening with the quantification of protein abundance.

A long-sought after goal has been T cell antigen discovery, whereby T cell receptors (TCRs) and their subunit pairs can be matched with peptide antigens presented on major histocompatibility complex (MHC) proteins. A comprehensive mapping and association between TCR and antigen pairs could enable us to better understand how to engineer cells to target molecular pathogens or unique tumor antigens. Recent work towards this goal has relied on fluorophore or heavy-metal labeled peptide-MHC (pMHC) ligands presented to libraries of T cells to identify specific TCR-pMHC pairs (Setliff et al., (2019). High-Throughput Mapping of B Cell Receptor Sequences to Antigen Specificity. Cell, 179(7), 1636-1646.; Joglekar, A. V., & Li, G. (2020). T cell antigen discovery. Nature Methods). Instead, by adopting PSP-seq techniques, libraries of T-cells can be presented to libraries of DNA-barcoded pMHC ligands to enable high-throughput discovery.

Finally, PSP-seq is a technique for single cell proteomics that is affordable and scalable. We hope that this technique will provide wider access for the scientific community to pursue complex proteomic studies.

### ADDITIONAL REFERENCES

1) Altayeb, O. A., Abdulaziz, M. S., & Osman, I. M. (2012). The role of CD20 and CD19 with their flow cytometric parameters in differentiation between Chronic Lymphocytic Leukaemia/Small Lymphocytic Lymphoma and other B-cell Non Hodgkin Lymphoma. Australian Journal of Basic and Applied Sciences, 6(10), 139-145
2) Jovanovic, D., Djurdjevic, P., Andjelkovic, N., & Zivic, L. (2014). Possible role of CD22, CD79b and CD20 expression in distinguishing small lymphocytic lymphoma from chronic lymphocytic leukemia. Contemporary Oncology, 18(1), 29-33.
3) Liu, Y., Beyer, A., & Aebersold, R. (2016). On the Dependency of Cellular Protein Levels on mRNA Abundance. Cell, 165(3), 535-550.

We claim:

1. A method for detecting a plurality of target molecules in, or on the surface of, exosomes, cells or vesicles in a sample, the method comprising:
(a) contacting the exosomes, cells or vesicles with a plurality of target molecule-binding agents, wherein each target molecule-binding agent comprises a nucleic acid barcode and optionally a unique molecular identifier (UMI), and wherein target molecule-binding agents that are specific to an identical target molecule share an identical nucleic acid barcode.

2. A method for diagnosing a disease or disorder, determining a patient's response to a therapy, and/or monitoring the patient for undesired side effects to the therapy, the method comprising:
(a) contacting exosomes, cells or vesicles with a plurality of target molecule-binding agents,
wherein each target molecule-binding agent comprises a nucleic acid barcode and optionally a unique molecular identifier (UMI),
wherein target molecule-binding agents that are specific to an identical target molecule share an identical nucleic acid barcode,
wherein the exosomes, cells or vesicles are isolated from a patient suffering from a disease or disorder,
wherein the plurality of target molecules comprise molecules that are markers indicative of the disease or disorder, efficacy of the therapy, and/or are indicative of undesirable side effects of the therapy, and
wherein expression levels of the target molecules determined in the patient are compared with expression levels of the corresponding target molecules determined in normal controls, and
thereby diagnosing a disease or disorder, e.g., cancer, autoimmune disease or inflammatory disease, determining the patient's response to a therapy, and/or monitoring for undesired side effects of a therapy.

3. The method of claim 2, wherein the patient is further treated with the same therapy, if the therapy is effective, but associated with little or no undesired side effects; or wherein the patient is further treated with a different therapy if the therapy is not effective and/or is associated with undesired side effects.

4. The method of claim 1, wherein each target molecule-binding agent further comprises a universal round 1 primer sequence at the 3′ end for a first round extension.

5. The method of claim 1, further comprising:
(b) dividing the exosomes, cells or vesicles, to which the target molecule-binding agents are bound, into at least two primary aliquots, the at least two primary aliquots comprising a first primary aliquot and a second primary aliquot;
(c) adding primary nucleic acid tags to the target molecule-binding agents in the at least two primary aliquots, wherein the primary nucleic acid tags added to the target molecule-binding agents in any one of the at least two primary aliquots are different from the primary nucleic acid tags added to the target molecule-binding agents in any one of the other primary aliquots;
(d) combining the at least two primary aliquots;
(e) dividing the combined primary aliquots into at least two secondary aliquots, the at least two secondary aliquots comprising a first secondary aliquot and a second secondary aliquot;
(f) adding secondary nucleic acid tags to the at least two secondary aliquots, wherein the secondary nucleic acid tags added to the target molecule-binding agents in any one of the at least two secondary aliquots are different from the secondary nucleic acid tags added to the target molecule-binding agents in any one of the other secondary aliquots; and
(g) repeating steps (d), (e), and (f) with the at least two secondary aliquots a number of times sufficient to generate a unique series of nucleic acid tags for each exosome, cell or vesicle in the sample.

6. The method of claim 5, wherein the primary nucleic acid tags, the secondary nucleic acid tags and/or subsequent nucleic acid tags are added by ligation reactions, polymerase extension reactions, and/or chemical syntheses.

7. The method of claim 6, wherein the nucleic acid tags are added by polymerase extension reaction,
wherein the nucleic acid barcode bound to each target molecule-binding agent is extended with one of the primary nucleic acid tags by contacting the exosomes, cells or vesicles with a strand displacing polymerase and a first DNA hairpin, wherein the first DNA hairpin comprises
(i) a first oligonucleotide comprising a sequence complementary to the universal round 1 primer sequence, and
(ii) a second oligonucleotide, wherein the second oligonucleotide comprises a third oligonucleotide comprising a primary nucleic acid tag, and a fourth oligonucleotide comprising a sequence complementary to the primary nucleic acid tag;
wherein the third oligonucleotide is located at the 5′ end of the second oligonucleotide, and the fourth oligonucleotide is located at the 3′ end of the second oligonucleotide; and
wherein the first oligonucleotide is fused to the 3′ end of the fourth oligonucleotide.

8. The method of claim 7, wherein each primary nucleic acid tag comprises a unique well-specific first round barcode sequence at the 5′ end and a universal round 2 primer sequence at the 3′ end.

9. The method of claim 7, wherein the first DNA hairpin is disabled at the end of each polymerase extension by removal of the first oligonucleotide from the first DNA hairpin using an exonuclease or by treating the first DNA hairpin with an enzyme that remove a unique base present at the junction of the first oligonucleotide and the fourth oligonucleotide.

10. The method of claim 9, further comprising adding secondary nucleic acid tags by polymerase extension reaction,
wherein the nucleic acid barcode bound to each target molecule-binding agent is further extended with one of the secondary nucleic acid tags by contacting the exosomes, cells or vesicles with a strand displacing polymerase and a second DNA hairpin wherein the second DNA hairpin comprises:
(i) a fifth oligonucleotide comprising a sequence complementarity to the universal round 2 primer sequence, and
(ii) a sixth oligonucleotide, wherein the sixth oligonucleotide further comprises a seventh oligonucleotide comprising a secondary nucleic acid tag, and an eighth oligonucleotide comprising a sequence encoding a sequence complementary to the secondary nucleic acid tag;
wherein the seventh oligonucleotide is located at the 5′ end of the sixth oligonucleotide, and the eighth oligonucleotide is located at the 3′ end of the sixth oligonucleotide; and
wherein the fifth oligonucleotide is fused to the 3′ end of the eighth oligonucleotide.

11. The method of claim 10, wherein each secondary nucleic acid tag comprises a unique well-specific second round barcode sequence at the 5′ end and a universal round 3 primer sequence at the 3′ end.

12. The method of claim 6, wherein the primary nucleic acid tags are added by ligation reaction in a first round splint-ligation reaction, wherein the primary nucleic acid tags are added to the 3′ end of the nucleic acid barcode bound to each target molecule-binding agent by contacting the exosomes, cells or vesicles with a ligase, a first-round oligonucleotide and a first-round splint sequence,
wherein the first-round oligonucleotide comprises a 5′ common region followed by a primary nucleic acid tag terminated by a 3′ universal round 2 sequence; and
wherein the first-round splint sequence comprises a region complementary to the universal round 1 sequence at 3′ end of the nucleic acid barcode bound to each target molecule-binding agent and a region complementary to the 5′ common region of the first-round oligonucleotide.

13. The method of claim 12, wherein the splint-ligation process is terminated at the end of the first-round splint-ligation reaction.

14. The method of claim 12, wherein secondary nucleic acid tags are added through a second round splint ligation reaction,
wherein the nucleic acid barcode bound to each target molecule-binding agent is further extended with a secondary nucleic acid tag by contacting the exosomes, cells or vesicles with a ligase, a second round oligonucleotide and a second round splint sequence,
wherein the second round oligonucleotide comprises a 5′ common region followed by a secondary nucleic acid tag terminated by a 3′ universal round 3 sequence or a universal PCR sequence; and
wherein the second round splint sequence comprises a region complimentary to the 3′ universal round 2 sequence of the first-round oligonucleotide, and a region complimentary to the 5′ common region of the second round oligonucleotide.

15. The method of claim 5, further comprising amplifying and sequencing the nucleic acid barcodes, the UMIs and the nucleic acid tags.

16. The method of claim 1, wherein the target molecules are proteins, sugar moieties, lipids, and/or polynucleotides.

17. The method of claim 1, wherein the target molecule-binding agents comprise an antibody, an antibody fragment, a peptide aptamer, lectins, a phage display system or a yeast display system.

18. The method of claim 1, wherein the nucleic acid barcode is a DNA-barcode.

19. The method of claim 1, wherein the detecting is at a single cell or a single exosome or a single vesicle level.

20. The method of claim 19, wherein each of the at least two primary aliquots consists of a single cell, a single exosome, or a single vesicle.


SEQUENCE LISTING (235 sequences)

SEQ ID NO: 1 - DNA, 14 bp, Artificial Sequence
  Description of Artificial Sequence Synthetic oligonucleotide
gggccttttggccc

SEQ ID NO: 2 - DNA, 76 bp, Artificial Sequence
  Description of Artificial Sequence Synthetic oligonucleotide
  Description of Combined DNA/RNA Molecule Synthetic oligonucleotide
  modified_base (58)..(58): ideoxyU
atacttattcttactaaattcagggccttttggccctgaatttagtaagaataagtauttgctaggacaaaaaaaa

SEQ ID NO: 3 - DNA, 12 bp, Artificial Sequence
  Description of Artificial Sequence Synthetic oligonucleotide
atacttattctt

SEQ ID NO: 4 - DNA, 10 bp, Artificial Sequence
  Description of Artificial Sequence Synthetic oligonucleotide
ttgctaggac

SEQ ID NO: 5 - DNA, 10 bp, Artificial Sequence
  Description of Artificial Sequence Synthetic oligonucleotide
actaaattca

SEQ ID NO: 6 - DNA, 22 bp, Artificial Sequence
  Description of Artificial Sequence Synthetic oligonucleotide
  Description of Combined DNA/RNA Molecule Synthetic oligonucleotide
  modified_base (22)..(22): ideoxyU
tgaatttagtaagaataagtau

SEQ ID NO: 7 - DNA, 15 bp, Artificial Sequence
  Description of Artificial Sequence Synthetic oligonucleotide
gtcaactctttagcg

SEQ ID NO: 8 - DNA, 15 bp, Artificial Sequence
  Description of Artificial Sequence Synthetic oligonucleotide
tgatggcctattggg

SEQ ID NO: 9 - DNA, 15 bp, Artificial Sequence
  Description of Artificial Sequence Synthetic oligonucleotide
ttccgcctctctttg

SEQ ID NO: 10 - DNA, 15 bp, Artificial Sequence
  Description of Artificial Sequence Synthetic oligonucleotide
agtaagttcagcgta

SEQ ID NO: 11 - DNA, 15 bp, Artificial Sequence
  Description of Artificial Sequence Synthetic oligonucleotide
ttcgccgcattgagt

SEQ ID NO: 12 - DNA, 15 bp, Artificial Sequence
  Description of Artificial Sequence Synthetic oligonucleotide
tgagaacgaccctaa

SEQ ID NO: 13 - DNA, 15 bp, Artificial Sequence
  Description of Artificial Sequence Synthetic oligonucleotide
tgcaattacccggat

SEQ ID NO: 14 - DNA, 15 bp, Artificial Sequence
  Description of Artificial Sequence Synthetic oligonucleotide
tgttcccgctcaact

SEQ ID NO: 15 - DNA, 15 bp, Artificial Sequence
  Description of Artificial Sequence Synthetic oligonucleotide
ctcctacctaaactg

SEQ ID NO: 16 - DNA, 15 bp, Artificial Sequence
  Description of Artificial Sequence Synthetic oligonucleotide
atcacatcgttgcca

SEQ ID NO: 17 - DNA, 15 bp, Artificial Sequence
  Description of Artificial Sequence Synthetic oligonucleotide
gtattccctcagtca

SEQ ID NO: 18 - DNA, 15 bp, Artificial Sequence
  Description of Artificial Sequence Synthetic oligonucleotide
agactaatagctgac

SEQ ID NO: 19 - DNA, 15 bp, Artificial Sequence
  Description of Artificial Sequence Synthetic oligonucleotide
ttctgggtccctaga

SEQ ID NO: 20 - DNA, 15 bp, Artificial Sequence
  Description of Artificial Sequence Synthetic oligonucleotide
aagttcactctttgc

SEQ ID NO: 21 - DNA, 15 bp, Artificial Sequence
  Description of Artificial Sequence Synthetic oligonucleotide
tacgatttgtcaggg

SEQ ID NO: 22 - DNA, 15 bp, Artificial Sequence
  Description of Artificial Sequence Synthetic oligonucleotide
tttcaacgccctttc

SEQ ID NO: 23 - DNA, 15 bp, Artificial Sequence
  Description of Artificial Sequence Synthetic oligonucleotide
tttgtcctgtacgcc

SEQ ID NO: 24 - DNA, 15 bp, Artificial Sequence
  Description of Artificial Sequence Synthetic oligonucleotide
aatagcgagcaagta

SEQ ID NO: 25 - DNA, 15 bp, Artificial Sequence
  Description of Artificial Sequence Synthetic oligonucleotide
tcagggtgtgctgta

SEQ ID NO: 26 - DNA, 15 bp, Artificial Sequence
  Description of Artificial Sequence Synthetic oligonucleotide
cagccattcattagg

SEQ ID NO: 27 - DNA, 15 bp, Artificial Sequence
  Description of Artificial Sequence Synthetic oligonucleotide
taactcagggcctat

SEQ ID NO: 28 - DNA, 15 bp, Artificial Sequence
  Description of Artificial Sequence Synthetic oligonucleotide
agctcagccagtatg

SEQ ID NO: 29 - DNA, 15 bp, Artificial Sequence
  Description of Artificial Sequence Synthetic oligonucleotide
cagccagtaagtcac

SEQ ID NO: 30 - DNA, 15 bp, Artificial Sequence
  Description of Artificial Sequence Synthetic oligonucleotide
caatcagacctatga

SEQ ID NO: 31 - DNA, 15 bp, Artificial Sequence
  Description of Artificial Sequence Synthetic oligonucleotide
ctgggcaattactcg

SEQ ID NO: 32 - DNA, 15 bp, Artificial Sequence
  Description of Artificial Sequence Synthetic oligonucleotide
tgaaggctcatttgt

SEQ ID NO: 33 - DNA, 15 bp, Artificial Sequence
  Description of Artificial Sequence Synthetic oligonucleotide
gcgcaacttgatgat

SEQ ID NO: 34 - DNA, 15 bp, Artificial Sequence
  Description of Artificial Sequence Synthetic oligonucleotide
ctcattgtaactcct

SEQ ID NO: 35 - DNA, 15 bp, Artificial Sequence
  Description of Artificial Sequence Synthetic oligonucleotide
acaatttgaacagcg

SEQ ID NO: 36 - DNA, 15 bp, Artificial Sequence
  Description of Artificial Sequence Synthetic oligonucleotide
cgtgtttgttcctca

SEQ ID NO: 37 - DNA, 15 bp, Artificial Sequence
  Description of Artificial Sequence Synthetic oligonucleotide
atggttcacgtaatc

SEQ ID NO: 38 - DNA, 15 bp, Artificial Sequence
  Description of Artificial Sequence Synthetic oligonucleotide
acagcgccgtattta

SEQ ID NO: 39 - DNA, 15 bp, Artificial Sequence
  Description of Artificial Sequence Synthetic oligonucleotide
gttgtccgacaatac

SEQ ID NO: 40 - DNA, 38 bp, Artificial Sequence
  Description of Artificial Sequence Synthetic oligonucleotide
gagttcgtgcacctaaacgtgattcagcatgcggctac

SEQ ID NO: 41 - DNA, 38 bp, Artificial Sequence
  Description of Artificial Sequence Synthetic oligonucleotide
gagttcgtgcacctaaaacatcgtcagcatgcggctac

SEQ ID NO: 42 - DNA, 38 bp, Artificial Sequence
  Description of Artificial Sequence Synthetic oligonucleotide
gagttcgtgcacctaatgcctaatcagcatgcggctac

SEQ ID NO: 43 - DNA, 38 bp, Artificial Sequence
  Description of Artificial Sequence Synthetic oligonucleotide
gagttcgtgcacctaagtggtcatcagcatgcggctac

SEQ ID NO: 44 - DNA, 38 bp, Artificial Sequence
  Description of Artificial Sequence Synthetic oligonucleotide
gagttcgtgcacctaaccactgttcagcatgcggctac

SEQ ID NO: 45 - DNA, 38 bp, Artificial Sequence
  Description of Artificial Sequence Synthetic oligonucleotide
gagttcgtgcacctaacattggctcagcatgcggctac

SEQ ID NO: 46 - DNA, 38 bp, Artificial Sequence
  Description of Artificial Sequence Synthetic oligonucleotide
gagttcgtgcacctacagatctgtcagcatgcggctac

SEQ ID NO: 47 - DNA, 38 bp, Artificial Sequence
  Description of Artificial Sequence Synthetic oligonucleotide
gagttcgtgcacctacatcaagttcagcatgcggctac

SEQ ID NO: 48 - DNA, 38 bp, Artificial Sequence
  Description of Artificial Sequence Synthetic oligonucleotide
gagttcgtgcacctacgctgatctcagcatgcggctac

SEQ ID NO: 49 - DNA, 38 bp, Artificial Sequence
  Description of Artificial Sequence Synthetic oligonucleotide
gagttcgtgcacctaacaagctatcagcatgcggctac

SEQ ID NO: 50 - DNA, 38 bp, Artificial Sequence
  Description of Artificial Sequence Synthetic oligonucleotide
gagttcgtgcacctactgtagcctcagcatgcggctac

SEQ ID NO: 51 - DNA, 38 bp, Artificial Sequence
  Description of Artificial Sequence Synthetic oligonucleotide
gagttcgtgcacctaagtacaagtcagcatgcggctac

SEQ ID NO: 52 - DNA, 38 bp, Artificial Sequence
  Description of Artificial Sequence Synthetic oligonucleotide
gagttcgtgcacctaaacaaccatcagcatgcggctac

SEQ ID NO: 53 - DNA, 38 bp, Artificial Sequence
  Description of Artificial Sequence Synthetic oligonucleotide
gagttcgtgcacctaaaccgagatcagcatgcggctac

SEQ ID NO: 54 - DNA, 38 bp, Artificial Sequence
  Description of Artificial Sequence Synthetic oligonucleotide
gagttcgtgcacctaaacgcttatcagcatgcggctac

SEQ ID NO: 55 - DNA, 38 bp, Artificial Sequence
  Description of Artificial Sequence Synthetic oligonucleotide
gagttcgtgcacctaaagacggatcagcatgcggctac

SEQ ID NO: 56 - DNA, 38 bp, Artificial Sequence
  Description of Artificial Sequence Synthetic oligonucleotide
gagttcgtgcacctaaaggtacatcagcatgcggctac

SEQ ID NO: 57 - DNA, 38 bp, Artificial Sequence
  Description of Artificial Sequence Synthetic oligonucleotide
gagttcgtgcacctaacacagaatcagcatgcggctac

SEQ ID NO: 58 - DNA, 38 bp, Artificial Sequence
  Description of Artificial Sequence Synthetic oligonucleotide
gagttcgtgcacctaacagcagatcagcatgcggctac

SEQ ID NO: 59 - DNA, 38 bp, Artificial Sequence
  Description of Artificial Sequence Synthetic oligonucleotide
gagttcgtgcacctaacctccaatcagcatgcggctac

SEQ ID NO: 60 - DNA, 38 bp, Artificial Sequence
  Description of Artificial Sequence Synthetic oligonucleotide
gagttcgtgcacctaacgctcgatcagcatgcggctac

SEQ ID NO: 61 - DNA, 38 bp, Artificial Sequence
  Description of Artificial Sequence Synthetic oligonucleotide
gagttcgtgcacctaacgtatcatcagcatgcggctac

SEQ ID NO: 62 - DNA, 38 bp, Artificial Sequence
  Description of Artificial Sequence Synthetic oligonucleotide
gagttcgtgcacctaactatgcatcagcatgcggctac

SEQ ID NO: 63 - DNA, 38 bp, Artificial Sequence
  Description of Artificial Sequence Synthetic oligonucleotide
gagttcgtgcacctaagagtcaatcagcatgcggctac

SEQ ID NO: 64 - DNA, 38 bp, Artificial Sequence
  Description of Artificial Sequence Synthetic oligonucleotide
gagttcgtgcacctaagatcgcatcagcatgcggctac

SEQ ID NO: 65 - DNA, 38 bp, Artificial Sequence
  Description of Artificial Sequence Synthetic oligonucleotide
gagttcgtgcacctaagcaggaatcagcatgcggctac

SEQ ID NO: 66 - DNA, 38 bp, Artificial Sequence
  Description of Artificial Sequence Synthetic oligonucleotide
gagttcgtgcacctaagtcactatcagcatgcggctac

SEQ ID NO: 67 - DNA, 38 bp, Artificial Sequence
  Description of Artificial Sequence Synthetic oligonucleotide
gagttcgtgcacctaatcctgtatcagcatgcggctac

SEQ ID NO: 68 - DNA, 38 bp, Artificial Sequence
  Description of Artificial Sequence Synthetic oligonucleotide
gagttcgtgcacctaattgaggatcagcatgcggctac

SEQ ID NO: 69 - DNA, 38 bp, Artificial Sequence
  Description of Artificial Sequence Synthetic oligonucleotide
gagttcgtgcacctacaaccacatcagcatgcggctac

SEQ ID NO: 70 - DNA, 38 bp, Artificial Sequence
  Description of Artificial Sequence Synthetic oligonucleotide
gagttcgtgcacctagactagtatcagcatgcggctac

SEQ ID NO: 71 - DNA, 38 bp, Artificial Sequence
  Description of Artificial Sequence Synthetic oligonucleotide
gagttcgtgcacctacaatggaatcagcatgcggctac

SEQ ID NO: 72 - DNA, 38 bp, Artificial Sequence
  Description of Artificial Sequence Synthetic oligonucleotide
gagttcgtgcacctacacttcgatcagcatgcggctac

SEQ ID NO: 73 - DNA, 38 bp, Artificial Sequence
  Description of Artificial Sequence Synthetic oligonucleotide
gagttcgtgcacctacagcgttatcagcatgcggctac

SEQ ID NO: 74 - DNA, 38 bp, Artificial Sequence
  Description of Artificial Sequence Synthetic oligonucleotide
gagttcgtgcacctacataccaatcagcatgcggctac

SEQ ID NO: 75 - DNA, 38 bp, Artificial Sequence
  Description of Artificial Sequence Synthetic oligonucleotide
gagttcgtgcacctaccagttcatcagcatgcggctac

SEQ ID NO: 76 - DNA, 38 bp, Artificial Sequence
  Description of Artificial Sequence Synthetic oligonucleotide
gagttcgtgcacctaccgaagtatcagcatgcggctac

SEQ ID NO: 77 - DNA, 38 bp, Artificial Sequence
  Description of Artificial Sequence Synthetic oligonucleotide
gagttcgtgcacctaccgtgagatcagcatgcggctac

SEQ ID NO: 78 - DNA, 38 bp, Artificial Sequence
  Description of Artificial Sequence Synthetic oligonucleotide
gagttcgtgcacctacctcctgatcagcatgcggctac

SEQ ID NO: 79 - DNA, 38 bp, Artificial Sequence
  Description of Artificial Sequence Synthetic oligonucleotide
gagttcgtgcacctacgaacttatcagcatgcggctac

SEQ ID NO: 80 - DNA, 38 bp, Artificial Sequence
  Description of Artificial Sequence Synthetic oligonucleotide
gagttcgtgcacctacgactggatcagcatgcggctac

SEQ ID NO: 81 - DNA, 38 bp, Artificial Sequence
  Description of Artificial Sequence Synthetic oligonucleotide
gagttcgtgcacctacgcatacatcagcatgcggctac

SEQ ID NO: 82 - DNA, 38 bp, Artificial Sequence
  Description of Artificial Sequence Synthetic oligonucleotide
gagttcgtgcacctactcaatgatcagcatgcggctac

SEQ ID NO: 83 - DNA, 38 bp, Artificial Sequence
  Description of Artificial Sequence Synthetic oligonucleotide
gagttcgtgcacctactgagccatcagcatgcggctac

SEQ ID NO: 84 - DNA, 38 bp, Artificial Sequence
  Description of Artificial Sequence Synthetic oligonucleotide
gagttcgtgcacctactggcatatcagcatgcggctac

SEQ ID NO: 85 - DNA, 38 bp, Artificial Sequence
  Description of Artificial Sequence Synthetic oligonucleotide
gagttcgtgcacctagaatctgatcagcatgcggctac

SEQ ID NO: 86 - DNA, 38 bp, Artificial Sequence
  Description of Artificial Sequence Synthetic oligonucleotide
gagttcgtgcacctacaagactatcagcatgcggctac

SEQ ID NO: 87 - DNA, 38 bp, Artificial Sequence
  Description of Artificial Sequence Synthetic oligonucleotide
gagttcgtgcacctagagctgaatcagcatgcggctac

SEQ ID NO: 88 - DNA, 38 bp, Artificial Sequence
  Description of Artificial Sequence Synthetic oligonucleotide
gagttcgtgcacctagatagacatcagcatgcggctac

SEQ ID NO: 89 - DNA, 38 bp, Artificial Sequence
  Description of Artificial Sequence Synthetic oligonucleotide
gagttcgtgcacctagccacatatcagcatgcggctac

SEQ ID NO: 90 - DNA, 38 bp, Artificial Sequence
  Description of Artificial Sequence Synthetic oligonucleotide
gagttcgtgcacctagcgagtaatcagcatgcggctac

SEQ ID NO: 91 - DNA, 38 bp, Artificial Sequence
  Description of Artificial Sequence Synthetic oligonucleotide
gagttcgtgcacctagctaacgatcagcatgcggctac

SEQ ID NO: 92 - DNA, 38 bp, Artificial Sequence
  Description of Artificial Sequence Synthetic oligonucleotide
gagttcgtgcacctagctcggtatcagcatgcggctac

SEQ ID NO: 93 - DNA, 38 bp, Artificial Sequence
  Description of Artificial Sequence Synthetic oligonucleotide
gagttcgtgcacctaggagaacatcagcatgcggctac

SEQ ID NO: 94 - DNA, 38 bp, Artificial Sequence
  Description of Artificial Sequence Synthetic oligonucleotide
gagttcgtgcacctaggtgcgaatcagcatgcggctac

SEQ ID NO: 95 - DNA, 38 bp, Artificial Sequence
  Description of Artificial Sequence Synthetic oligonucleotide
gagttcgtgcacctagtacgcaatcagcatgcggctac

SEQ ID NO: 96 - DNA, 38 bp, Artificial Sequence
  Description of Artificial Sequence Synthetic oligonucleotide
gagttcgtgcacctagtcgtagatcagcatgcggctac

SEQ ID NO: 97 - DNA, 38 bp, Artificial Sequence
  Description of Artificial Sequence Synthetic oligonucleotide
gagttcgtgcacctagtctgtcatcagcatgcggctac

SEQ ID NO: 98 - DNA, 38 bp, Artificial Sequence
  Description of Artificial Sequence Synthetic oligonucleotide
gagttcgtgcacctagtgttctatcagcatgcggctac

SEQ ID NO: 99 - DNA, 38 bp, Artificial Sequence
  Description of Artificial Sequence Synthetic oligonucleotide
gagttcgtgcacctataggatgatcagcatgcggctac

SEQ ID NO: 100 - DNA, 38 bp, Artificial Sequence
  Description of Artificial Sequence Synthetic oligonucleotide
gagttcgtgcacctatatcagcatcagcatgcggctac

SEQ ID NO: 101 - DNA, 38 bp, Artificial Sequence
  Description of Artificial Sequence Synthetic oligonucleotide
gagttcgtgcacctatccgtctatcagcatgcggctac

SEQ ID NO: 102 - DNA, 38 bp, Artificial Sequence
  Description of Artificial Sequence Synthetic oligonucleotide
gagttcgtgcacctatcttcacatcagcatgcggctac

SEQ ID NO: 103 - DNA, 38 bp, Artificial Sequence
  Description of Artificial Sequence Synthetic oligonucleotide
gagttcgtgcacctatgaagagatcagcatgcggctac

SEQ ID NO: 104 - DNA, 38 bp, Artificial Sequence
  Description of Artificial Sequence Synthetic oligonucleotide
gagttcgtgcacctatggaacaatcagcatgcggctac

SEQ ID NO: 105 - DNA, 38 bp, Artificial Sequence
  Description of Artificial Sequence Synthetic oligonucleotide
gagttcgtgcacctatggcttcatcagcatgcggctac

SEQ ID NO: 106 - DNA, 38 bp, Artificial Sequence
  Description of Artificial Sequence Synthetic oligonucleotide
gagttcgtgcacctatggtggtatcagcatgcggctac

SEQ ID NO: 107 - DNA, 38 bp, Artificial Sequence
  Description of Artificial Sequence Synthetic oligonucleotide
gagttcgtgcacctattcacgcatcagcatgcggctac

SEQ ID NO: 108 - DNA, 38 bp, Artificial Sequence
  Description of Artificial Sequence Synthetic oligonucleotide
gagttcgtgcacctaaactcacctcagcatgcggctac

SEQ ID NO: 109 - DNA, 38 bp, Artificial Sequence
  Description of Artificial Sequence Synthetic oligonucleotide
gagttcgtgcacctaaagagatctcagcatgcggctac

SEQ ID NO: 110 - DNA, 38 bp, Artificial Sequence
  Description of Artificial Sequence Synthetic oligonucleotide
gagttcgtgcacctaaaggacactcagcatgcggctac

SEQ ID NO: 111 - DNA, 38 bp, Artificial Sequence
  Description of Artificial Sequence Synthetic oligonucleotide
gagttcgtgcacctaaatccgtctcagcatgcggctac

SEQ ID NO: 112 - DNA, 38 bp, Artificial Sequence
  Description of Artificial Sequence Synthetic oligonucleotide
gagttcgtgcacctaaatgttgctcagcatgcggctac

SEQ ID NO: 113 - DNA, 38 bp, Artificial Sequence
  Description of Artificial Sequence Synthetic oligonucleotide
gagttcgtgcacctaacacgacctcagcatgcggctac

SEQ ID NO: 114 - DNA, 38 bp, Artificial Sequence
  Description of Artificial Sequence Synthetic oligonucleotide
gagttcgtgcacctaacagattctcagcatgcggctac

SEQ ID NO: 115 - DNA, 38 bp, Artificial Sequence
  Description of Artificial Sequence Synthetic oligonucleotide
gagttcgtgcacctaagatgtactcagcatgcggctac

SEQ ID NO: 116 - DNA, 38 bp, Artificial Sequence
  Description of Artificial Sequence Synthetic oligonucleotide
gagttcgtgcacctaagcacctctcagcatgcggctac

SEQ ID NO: 117 - DNA, 38 bp, Artificial Sequence
  Description of Artificial Sequence Synthetic oligonucleotide
gagttcgtgcacctaagccatgctcagcatgcggctac

SEQ ID NO: 118 - DNA, 38 bp, Artificial Sequence
  Description of Artificial Sequence Synthetic oligonucleotide
gagttcgtgcacctaaggctaactcagcatgcggctac

SEQ ID NO: 119 - DNA, 38 bp, Artificial Sequence
  Description of Artificial Sequence Synthetic oligonucleotide
gagttcgtgcacctaatagcgactcagcatgcggctac

SEQ ID NO: 120 - DNA, 38 bp, Artificial Sequence
  Description of Artificial Sequence Synthetic oligonucleotide
gagttcgtgcacctaatcattcctcagcatgcggctac

SEQ ID NO: 121 - DNA, 38 bp, Artificial Sequence
  Description of Artificial Sequence Synthetic oligonucleotide
gagttcgtgcacctaattggctctcagcatgcggctac

SEQ ID NO: 122 - DNA, 38 bp, Artificial Sequence
  Description of Artificial Sequence Synthetic oligonucleotide
gagttcgtgcacctacaaggagctcagcatgcggctac

SEQ ID NO: 123 - DNA, 38 bp, Artificial Sequence
  Description of Artificial Sequence Synthetic oligonucleotide
gagttcgtgcacctacaccttactcagcatgcggctac

SEQ ID NO: 124 - DNA, 38 bp, Artificial Sequence
  Description of Artificial Sequence Synthetic oligonucleotide
gagttcgtgcacctaccatcctctcagcatgcggctac

SEQ ID NO: 125 - DNA, 38 bp, Artificial Sequence
  Description of Artificial Sequence Synthetic oligonucleotide
gagttcgtgcacctaccgacaactcagcatgcggctac

SEQ ID NO: 126 - DNA, 38 bp, Artificial Sequence
  Description of Artificial Sequence Synthetic oligonucleotide
gagttcgtgcacctacctaatcctcagcatgcggctac

SEQ ID NO: 127 - DNA, 38 bp, Artificial Sequence
  Description of Artificial Sequence Synthetic oligonucleotide
gagttcgtgcacctacctctatctcagcatgcggctac

SEQ ID NO: 128 - DNA, 38 bp, Artificial Sequence
  Description of Artificial Sequence Synthetic oligonucleotide
gagttcgtgcacctacgacacactcagcatgcggctac

SEQ ID NO: 129 - DNA, 38 bp, Artificial Sequence
  Description of Artificial Sequence Synthetic oligonucleotide
gagttcgtgcacctacggattgctcagcatgcggctac

SEQ ID NO: 130 - DNA, 38 bp, Artificial Sequence
  Description of Artificial Sequence Synthetic oligonucleotide
gagttcgtgcacctactaaggtctcagcatgcggctac

SEQ ID NO: 131 - DNA, 38 bp, Artificial Sequence
  Description of Artificial Sequence Synthetic oligonucleotide
gagttcgtgcacctagaacaggctcagcatgcggctac

SEQ ID NO: 132 - DNA, 38 bp, Artificial Sequence
  Description of Artificial Sequence Synthetic oligonucleotide
gagttcgtgcacctagacagtgctcagcatgcggctac

SEQ ID NO: 133 - DNA, 38 bp, Artificial Sequence
  Description of Artificial Sequence Synthetic oligonucleotide
gagttcgtgcacctagagttagctcagcatgcggctac

SEQ ID NO: 134 - DNA, 38 bp, Artificial Sequence
  Description of Artificial Sequence Synthetic oligonucleotide
gagttcgtgcacctagatgaatctcagcatgcggctac

SEQ ID NO: 135 - DNA, 38 bp, Artificial Sequence
  Description of Artificial Sequence Synthetic oligonucleotide
gagttcgtgcacctagccaagactcagcatgcggctac

SEQ ID NO: 136 - DNA, 53 bp, Artificial Sequence
  Description of Artificial Sequence Synthetic oligonucleotide
gctttgtagccggtgaacgtgatagatcggaagagcgtcgtgtagggaaagag

SEQ ID NO: 137 - DNA, 53 bp, Artificial Sequence
  Description of Artificial Sequence Synthetic oligonucleotide
gctttgtagccggtgaaacatcgagatcggaagagcgtcgtgtagggaaagag

SEQ ID NO: 138 - DNA, 53 bp, Artificial Sequence
  Description of Artificial Sequence Synthetic oligonucleotide
gctttgtagccggtgatgcctaaagatcggaagagcgtcgtgtagggaaagag

SEQ ID NO: 139 - DNA, 53 bp, Artificial Sequence
  Description of Artificial Sequence Synthetic oligonucleotide
gctttgtagccggtgagtggtcaagatcggaagagcgtcgtgtagggaaagag

SEQ ID NO: 140 - DNA, 53 bp, Artificial Sequence
  Description of Artificial Sequence Synthetic oligonucleotide
gctttgtagccggtgaccactgtagatcggaagagcgtcgtgtagggaaagag

SEQ ID NO: 141 - DNA, 53 bp, Artificial Sequence
  Description of Artificial Sequence Synthetic oligonucleotide
gctttgtagccggtgacattggcagatcggaagagcgtcgtgtagggaaagag

SEQ ID NO: 142 - DNA, 53 bp, Artificial Sequence
  Description of Artificial Sequence Synthetic oligonucleotide
gctttgtagccggtgcagatctgagatcggaagagcgtcgtgtagggaaagag

SEQ ID NO: 143 - DNA, 53 bp, Artificial Sequence
  Description of Artificial Sequence Synthetic oligonucleotide
gctttgtagccggtgcatcaagtagatcggaagagcgtcgtgtagggaaagag

SEQ ID NO: 144 - DNA, 53 bp, Artificial Sequence
  Description of Artificial Sequence Synthetic oligonucleotide
gctttgtagccggtgcgctgatcagatcggaagagcgtcgtgtagggaaagag

SEQ ID NO: 145 - DNA, 53 bp, Artificial Sequence
  Description of Artificial Sequence Synthetic oligonucleotide
gctttgtagccggtgacaagctaagatcggaagagcgtcgtgtagggaaagag

SEQ ID NO: 146 - DNA, 53 bp, Artificial Sequence
  Description of Artificial Sequence Synthetic oligonucleotide
gctttgtagccggtgctgtagccagatcggaagagcgtcgtgtagggaaagag

SEQ ID NO: 147 - DNA, 53 bp, Artificial Sequence
  Description of Artificial Sequence Synthetic oligonucleotide
gctttgtagccggtgagtacaagagatcggaagagcgtcgtgtagggaaagag

SEQ ID NO: 148 - DNA, 53 bp, Artificial Sequence
  Description of Artificial Sequence Synthetic oligonucleotide
gctttgtagccggtgaacaaccaagatcggaagagcgtcgtgtagggaaagag

SEQ ID NO: 149 - DNA, 53 bp, Artificial Sequence
  Description of Artificial Sequence Synthetic oligonucleotide
gctttgtagccggtgaaccgagaagatcggaagagcgtcgtgtagggaaagag

SEQ ID NO: 150 - DNA, 53 bp, Artificial Sequence
  Description of Artificial Sequence Synthetic oligonucleotide
gctttgtagccggtgaacgcttaagatcggaagagcgtcgtgtagggaaagag

SEQ ID NO: 151 - DNA, 53 bp, Artificial Sequence
  Description of Artificial Sequence Synthetic oligonucleotide
gctttgtagccggtgaagacggaagatcggaagagcgtcgtgtagggaaagag

SEQ ID NO: 152 - DNA, 53 bp, Artificial Sequence
  Description of Artificial Sequence Synthetic oligonucleotide
gctttgtagccggtgaaggtacaagatcggaagagcgtcgtgtagggaaagag

SEQ ID NO: 153 - DNA, 53 bp, Artificial Sequence
  Description of Artificial Sequence Synthetic oligonucleotide
gctttgtagccggtgacacagaaagatcggaagagcgtcgtgtagggaaagag

SEQ ID NO: 154 - DNA, 53 bp, Artificial Sequence
  Description of Artificial Sequence Synthetic oligonucleotide
gctttgtagccggtgacagcagaagatcggaagagcgtcgtgtagggaaagag

SEQ ID NO: 155 - DNA, 53 bp, Artificial Sequence
  Description of Artificial Sequence Synthetic oligonucleotide
gctttgtagccggtgacctccaaagatcggaagagcgtcgtgtagggaaagag

SEQ ID NO: 156 - DNA, 53 bp, Artificial Sequence
  Description of Artificial Sequence Synthetic oligonucleotide
gctttgtagccggtgacgctcgaagatcggaagagcgtcgtgtagggaaagag

SEQ ID NO: 157 - DNA, 53 bp, Artificial Sequence
  Description of Artificial Sequence Synthetic oligonucleotide
gctttgtagccggtgacgtatcaagatcggaagagcgtcgtgtagggaaagag

SEQ ID NO: 158 - DNA, 53 bp, Artificial Sequence
  Description of Artificial Sequence Synthetic oligonucleotide
gctttgtagccggtgactatgcaagatcggaagagcgtcgtgtagggaaagag

SEQ ID NO: 159 - DNA, 53 bp, Artificial Sequence
  Description of Artificial Sequence Synthetic oligonucleotide
gctttgtagccggtgagagtcaaagatcggaagagcgtcgtgtagggaaagag

SEQ ID NO: 160 - DNA, 53 bp, Artificial Sequence
  Description of Artificial Sequence Synthetic oligonucleotide
gctttgtagccggtgagatcgcaagatcggaagagcgtcgtgtagggaaagag

SEQ ID NO: 161 - DNA, 53 bp, Artificial Sequence
  Description of Artificial Sequence Synthetic oligonucleotide
gctttgtagccggtgagcaggaaagatcggaagagcgtcgtgtagggaaagag

SEQ ID NO: 162 - DNA, 53 bp, Artificial Sequence
  Description of Artificial Sequence Synthetic oligonucleotide
gctttgtagccggtgagtcactaagatcggaagagcgtcgtgtagggaaagag

SEQ ID NO: 163 - DNA, 53 bp, Artificial Sequence
  Description of Artificial Sequence Synthetic oligonucleotide
gctttgtagccggtgatcctgtaagatcggaagagcgtcgtgtagggaaagag

SEQ ID NO: 164 - DNA, 53 bp, Artificial Sequence
  Description of Artificial Sequence Synthetic oligonucleotide
gctttgtagccggtgattgaggaagatcggaagagcgtcgtgtagggaaagag

SEQ ID NO: 165 - DNA, 53 bp, Artificial Sequence
  Description of Artificial Sequence Synthetic oligonucleotide
gctttgtagccggtgcaaccacaagatcggaagagcgtcgtgtagggaaagag

SEQ ID NO: 166 - DNA, 53 bp, Artificial Sequence
  Description of Artificial Sequence Synthetic oligonucleotide
gctttgtagccggtggactagtaagatcggaagagcgtcgtgtagggaaagag

SEQ ID NO: 167 - DNA, 53 bp, Artificial Sequence
  Description of Artificial Sequence Synthetic oligonucleotide
gctttgtagccggtgcaatggaaagatcggaagagcgtcgtgtagggaaagag

SEQ ID NO: 168 - DNA, 53 bp, Artificial Sequence
  Description of Artificial Sequence Synthetic oligonucleotide
gctttgtagccggtgcacttcgaagatcggaagagcgtcgtgtagggaaagag

SEQ ID NO: 169 - DNA, 53 bp, Artificial Sequence
  Description of Artificial Sequence Synthetic oligonucleotide
gctttgtagccggtgcagcgttaagatcggaagagcgtcgtgtagggaaagag

SEQ ID NO: 170 - DNA, 53 bp, Artificial Sequence
  Description of Artificial Sequence Synthetic oligonucleotide
gctttgtagccggtgcataccaaagatcggaagagcgtcgtgtagggaaagag

SEQ ID NO: 171 - DNA, 53 bp, Artificial Sequence
  Description of Artificial Sequence Synthetic oligonucleotide
gctttgtagccggtgccagttcaagatcggaagagcgtcgtgtagggaaagag

SEQ ID NO: 172 - DNA, 53 bp, Artificial Sequence
  Description of Artificial Sequence Synthetic oligonucleotide
gctttgtagccggtgccgaagtaagatcggaagagcgtcgtgtagggaaagag

SEQ ID NO: 173 - DNA, 53 bp, Artificial Sequence
  Description of Artificial Sequence Synthetic oligonucleotide
gctttgtagccggtgccgtgagaagatcggaagagcgtcgtgtagggaaagag

SEQ ID NO: 174 - DNA, 53 bp, Artificial Sequence
  Description of Artificial Sequence Synthetic oligonucleotide
gctttgtagccggtgcctcctgaagatcggaagagcgtcgtgtagggaaagag

SEQ ID NO: 175 - DNA, 53 bp, Artificial Sequence
  Description of Artificial Sequence Synthetic oligonucleotide
gctttgtagccggtgcgaacttaagatcggaagagcgtcgtgtagggaaagag

SEQ ID NO: 176 - DNA, 53 bp, Artificial Sequence
  Description of Artificial Sequence Synthetic oligonucleotide
gctttgtagccggtgcgactggaagatcggaagagcgtcgtgtagggaaagag

SEQ ID NO: 177 - DNA, 53 bp, Artificial Sequence
  Description of Artificial Sequence Synthetic oligonucleotide
gctttgtagccggtgcgcatacaagatcggaagagcgtcgtgtagggaaagag

SEQ ID NO: 178 - DNA, 53 bp, Artificial Sequence
  Description of Artificial Sequence Synthetic oligonucleotide
gctttgtagccggtgctcaatgaagatcggaagagcgtcgtgtagggaaagag

SEQ ID NO: 179 - DNA, 53 bp, Artificial Sequence
  Description of Artificial Sequence Synthetic oligonucleotide
gctttgtagccggtgctgagccaagatcggaagagcgtcgtgtagggaaagag

SEQ ID NO: 180 - DNA, 53 bp, Artificial Sequence
  Description of Artificial Sequence Synthetic oligonucleotide
gctttgtagccggtgctggcataagatcggaagagcgtcgtgtagggaaagag

SEQ ID NO: 181 - DNA, 53 bp, Artificial Sequence
  Description of Artificial Sequence Synthetic oligonucleotide
gctttgtagccggtggaatctgaagatcggaagagcgtcgtgtagggaaagag

SEQ ID NO: 182 - DNA, 53 bp, Artificial Sequence
  Description of Artificial Sequence Synthetic oligonucleotide
gctttgtagccggtgcaagactaagatcggaagagcgtcgtgtagggaaagag

SEQ ID NO: 183 - DNA, 53 bp, Artificial Sequence
  Description of Artificial Sequence Synthetic oligonucleotide
gctttgtagccggtggagctgaaagatcggaagagcgtcgtgtagggaaagag

SEQ ID NO: 184 - DNA, 53 bp, Artificial Sequence
  Description of Artificial Sequence Synthetic oligonucleotide
gctttgtagccggtggatagacaagatcggaagagcgtcgtgtagggaaagag

SEQ ID NO: 185 - DNA, 53 bp, Artificial Sequence
  Description of Artificial Sequence Synthetic oligonucleotide
gctttgtagccggtggccacataagatcggaagagcgtcgtgtagggaaagag

SEQ ID NO: 186 - DNA, 53 bp, Artificial Sequence
  Description of Artificial Sequence Synthetic oligonucleotide
gctttgtagccggtggcgagtaaagatcggaagagcgtcgtgtagggaaagag

SEQ ID NO: 187 - DNA, 53 bp, Artificial Sequence
  Description of Artificial Sequence Synthetic oligonucleotide
gctttgtagccggtggctaacgaagatcggaagagcgtcgtgtagggaaagag

SEQ ID NO: 188 - DNA, 53 bp, Artificial Sequence
  Description of Artificial Sequence Synthetic oligonucleotide
gctttgtagccggtggctcggtaagatcggaagagcgtcgtgtagggaaagag

SEQ ID NO: 189 - DNA, 53 bp, Artificial Sequence
  Description of Artificial Sequence Synthetic oligonucleotide
gctttgtagccggtgggagaacaagatcggaagagcgtcgtgtagggaaagag

SEQ ID NO: 190 - DNA, 53 bp, Artificial Sequence
  Description of Artificial Sequence Synthetic oligonucleotide
gctttgtagccggtgggtgcgaaagatcggaagagcgtcgtgtagggaaagag

SEQ ID NO: 191 - DNA, 53 bp, Artificial Sequence
  Description of Artificial Sequence Synthetic oligonucleotide
gctttgtagccggtggtacgcaaagatcggaagagcgtcgtgtagggaaagag

SEQ ID NO: 192 - DNA, 53 bp, Artificial Sequence
  Description of Artificial Sequence Synthetic oligonucleotide
gctttgtagccggtggtcgtagaagatcggaagagcgtcgtgtagggaaagag

SEQ ID NO: 193 - DNA, 53 bp, Artificial Sequence
  Description of Artificial Sequence Synthetic oligonucleotide
gctttgtagccggtggtctgtcaagatcggaagagcgtcgtgtagggaaagag

SEQ ID NO: 194 - DNA, 53 bp, Artificial Sequence
  Description of Artificial Sequence Synthetic oligonucleotide
gctttgtagccggtggtgttctaagatcggaagagcgtcgtgtagggaaagag

SEQ ID NO: 195 - DNA, 53 bp, Artificial Sequence
  Description of Artificial Sequence Synthetic oligonucleotide
gctttgtagccggtgtaggatgaagatcggaagagcgtcgtgtagggaaagag

SEQ ID NO: 196 - DNA, 53 bp, Artificial Sequence
  Description of Artificial Sequence Synthetic oligonucleotide
gctttgtagccggtgtatcagcaagatcggaagagcgtcgtgtagggaaagag

SEQ ID NO: 197 - DNA, 53 bp, Artificial Sequence
  Description of Artificial Sequence Synthetic oligonucleotide
gctttgtagccggtgtccgtctaagatcggaagagcgtcgtgtagggaaagag

SEQ ID NO: 198 - DNA, 53 bp, Artificial Sequence
  Description of Artificial Sequence Synthetic oligonucleotide
gctttgtagccggtgtcttcacaagatcggaagagcgtcgtgtagggaaagag

SEQ ID NO: 199 - DNA, 53 bp, Artificial Sequence
  Description of Artificial Sequence Synthetic oligonucleotide
gctttgtagccggtgtgaagagaagatcggaagagcgtcgtgtagggaaagag

SEQ ID NO: 200 - DNA, 53 bp, Artificial Sequence
  Description of Artificial Sequence Synthetic oligonucleotide
gctttgtagccggtgtggaacaaagatcggaagagcgtcgtgtagggaaagag

SEQ ID NO: 201 - DNA, 53 bp, Artificial Sequence
  Description of Artificial Sequence Synthetic oligonucleotide
gctttgtagccggtgtggcttcaagatcggaagagcgtcgtgtagggaaagag

SEQ ID NO: 202 - DNA, 53 bp, Artificial Sequence
  Description of Artificial Sequence Synthetic oligonucleotide
gctttgtagccggtgtggtggtaagatcggaagagcgtcgtgtagggaaagag

SEQ ID NO: 203 - DNA, 53 bp, Artificial Sequence
  Description of Artificial Sequence Synthetic oligonucleotide
gctttgtagccggtgttcacgcaagatcggaagagcgtcgtgtagggaaagag

SEQ ID NO: 204 - DNA, 53 bp, Artificial Sequence
  Description of Artificial Sequence Synthetic oligonucleotide
gctttgtagccggtgaactcaccagatcggaagagcgtcgtgtagggaaagag

SEQ ID NO: 205 - DNA, 53 bp, Artificial Sequence
  Description of Artificial Sequence Synthetic oligonucleotide
gctttgtagccggtgaagagatcagatcggaagagcgtcgtgtagggaaagag

SEQ ID NO: 206 - DNA, 53 bp, Artificial Sequence
  Description of Artificial Sequence Synthetic oligonucleotide
gctttgtagccggtgaaggacacagatcggaagagcgtcgtgtagggaaagag

SEQ ID NO: 207 - DNA, 53 bp, Artificial Sequence
  Description of Artificial Sequence Synthetic oligonucleotide
gctttgtagccggtgaatccgtcagatcggaagagcgtcgtgtagggaaagag

SEQ ID NO: 208 - DNA, 53 bp, Artificial Sequence
  Description of Artificial Sequence Synthetic oligonucleotide
gctttgtagccggtgaatgttgcagatcggaagagcgtcgtgtagggaaagag

SEQ ID NO: 209 - DNA, 53 bp, Artificial Sequence
  Description of Artificial Sequence Synthetic oligonucleotide
gctttgtagccggtgacacgaccagatcggaagagcgtcgtgtagggaaagag

SEQ ID NO: 210 - DNA, 53 bp, Artificial Sequence
  Description of Artificial Sequence Synthetic oligonucleotide
gctttgtagccggtgacagattcagatcggaagagcgtcgtgtagggaaagag

SEQ ID NO: 211 - DNA, 53 bp, Artificial Sequence
  Description of Artificial Sequence Synthetic oligonucleotide
gctttgtagccggtgagatgtacagatcggaagagcgtcgtgtagggaaagag

SEQ ID NO: 212 - DNA, 53 bp, Artificial Sequence
  Description of Artificial Sequence Synthetic oligonucleotide
gctttgtagccggtgagcacctcagatcggaagagcgtcgtgtagggaaagag

SEQ ID NO: 213 - DNA, 53 bp, Artificial Sequence
  Description of Artificial Sequence Synthetic oligonucleotide
gctttgtagccggtgagccatgcagatcggaagagcgtcgtgtagggaaagag

SEQ ID NO: 214 - DNA, 53 bp, Artificial Sequence
  Description of Artificial Sequence Synthetic oligonucleotide
gctttgtagccggtgaggctaacagatcggaagagcgtcgtgtagggaaagag

SEQ ID NO: 215 - DNA, 53 bp, Artificial Sequence
  Description of Artificial Sequence Synthetic oligonucleotide
gctttgtagccggtgatagcgacagatcggaagagcgtcgtgtagggaaagag

SEQ ID NO: 216 - DNA, 53 bp, Artificial Sequence
  Description of Artificial Sequence Synthetic oligonucleotide
gctttgtagccggtgatcattccagatcggaagagcgtcgtgtagggaaagag

SEQ ID NO: 217 - DNA, 53 bp, Artificial Sequence
  Description of Artificial Sequence Synthetic oligonucleotide
gctttgtagccggtgattggctcagatcggaagagcgtcgtgtagggaaagag

SEQ ID NO: 218 - DNA, 53 bp, Artificial Sequence
  Description of Artificial Sequence Synthetic oligonucleotide
gctttgtagccggtgcaaggagcagatcggaagagcgtcgtgtagggaaagag

SEQ ID NO: 219 - DNA, 53 bp, Artificial Sequence
  Description of Artificial Sequence Synthetic oligonucleotide
gctttgtagccggtgcaccttacagatcggaagagcgtcgtgtagggaaagag

SEQ ID NO: 220 - DNA, 53 bp, Artificial Sequence
  Description of Artificial Sequence Synthetic oligonucleotide
gctttgtagccggtgccatcctcagatcggaagagcgtcgtgtagggaaagag

SEQ ID NO: 221 - DNA, 53 bp, Artificial Sequence
  Description of Artificial Sequence Synthetic oligonucleotide
gctttgtagccggtgccgacaacagatcggaagagcgtcgtgtagggaaagag

SEQ ID NO: 222 - DNA, 53 bp, Artificial Sequence
  Description of Artificial Sequence Synthetic oligonucleotide
gctttgtagccggtgcctaatccagatcggaagagcgtcgtgtagggaaagag

SEQ ID NO: 223 - DNA, 53 bp, Artificial Sequence
  Description of Artificial Sequence Synthetic oligonucleotide
gctttgtagccggtgcctctatcagatcggaagagcgtcgtgtagggaaagag

SEQ ID NO: 224 - DNA, 53 bp, Artificial Sequence
  Description of Artificial Sequence Synthetic oligonucleotide
gctttgtagccggtgcgacacacagatcggaagagcgtcgtgtagggaaagag

SEQ ID NO: 225 - DNA, 53 bp, Artificial Sequence
  Description of Artificial Sequence Synthetic oligonucleotide
gctttgtagccggtgcggattgcagatcggaagagcgtcgtgtagggaaagag

SEQ ID NO: 226 - DNA, 53 bp, Artificial Sequence
  Description of Artificial Sequence Synthetic oligonucleotide
gctttgtagccggtgctaaggtcagatcggaagagcgtcgtgtagggaaagag

SEQ ID NO: 227 - DNA, 53 bp, Artificial Sequence
  Description of Artificial Sequence Synthetic oligonucleotide
gctttgtagccggtggaacaggcagatcggaagagcgtcgtgtagggaaagag

SEQ ID NO: 228 - DNA, 53 bp, Artificial Sequence
  Description of Artificial Sequence Synthetic oligonucleotide
gctttgtagccggtggacagtgcagatcggaagagcgtcgtgtagggaaagag

SEQ ID NO: 229 - DNA, 53 bp, Artificial Sequence
  Description of Artificial Sequence Synthetic oligonucleotide
gctttgtagccggtggagttagcagatcggaagagcgtcgtgtagggaaagag

SEQ ID NO: 230 - DNA, 53 bp, Artificial Sequence
  Description of Artificial Sequence Synthetic oligonucleotide
gctttgtagccggtggatgaatcagatcggaagagcgtcgtgtagggaaagag

SEQ ID NO: 231 - DNA, 53 bp, Artificial Sequence
  Description of Artificial Sequence Synthetic oligonucleotide
gctttgtagccggtggccaagacagatcggaagagcgtcgtgtagggaaagag

SEQ ID NO: 232 - DNA, 29 bp, Artificial Sequence
  Description of Artificial Sequence Synthetic oligonucleotide
taggtgcacgaactcttgctaggaccggc

SEQ ID NO: 233 - DNA, 30 bp, Artificial Sequence
  Description of Artificial Sequence Synthetic oligonucleotide
caccggctacaaagcgtagccgcatgctga

SEQ ID NO: 234 - DNA, 29 bp, Artificial Sequence
  Description of Artificial Sequence Synthetic oligonucleotide
gccggtcctagcaagagttcgtgcaccta

SEQ ID NO: 235 - DNA, 30 bp, Artificial Sequence
  Description of Artificial Sequence Synthetic oligonucleotide
tcagcatgcggctacgctttgtagccggtg